(19) United States
(12) Patent Application Publication
(10) Pub. No.: US 2025/0054637 A1
(43) Pub. Date: Feb. 13, 2025

(54) Machine Learning-Based Phenotypic Age and Phenotypic Age Acceleration/Deceleration Prediction Tool for Pets

(71) Applicant: Hill's Pet Nutrition, Inc., Overland Park, KS (US)

(72) Inventors: Susan WERNIMONT, Lawrence, KS (US); Robin Thompson, Ryron (GB); Daniel Horschler, Lawrence, KS (US); Kathy Gross, Topeka, KS (US)

(73) Assignee: Hill's Pet Nutrition, Inc., Overland Park, KS (US)

(21) Appl. No.: 18/800,955

(22) Filed: Aug. 12, 2024

Related U.S. Application Data
(60) Provisional application No. 63/532,360, filed on Aug. 12, 2023.

(51) Int. Cl. G16H 50/30; G16B 25/10; G16H 10/60; G16H 40/67

(52) U.S. Cl. CPC G16H 50/30; G16B 25/10; G16H 10/60; G16H 40/67

(57) ABSTRACT

The disclosure provides a system for generating a multi-component aging index for an individual companion animal comprising digital biomarkers, biological biomarkers and subjective assessment methods to predict phenotypic age and phenotypic age acceleration/deceleration (phenotypic age above or below chronological age) in dogs and cats. The disclosure also provides a method for decelerating phenotypic aging of a companion animal in need thereof. The method comprises determining an index according to an aspect of the disclosure and providing personalized health, diet and nutrition measures to the companion animal according to the index for that companion animal. The method can also be considered to be a method of ameliorating accelerated aging in the animal. In some embodiments, the personalized health measure comprises a diet that decelerates phenotypic aging and/or ameliorates accelerated phenotypic aging.

### CROSS-REFERENCE TO RELATED APPLICATION

This application claims the benefit of U.S. provisional application No. 63/532,360, which was filed Aug. 12, 2023, is titled “Machine Learning-Based Phenotypic Age and Phenotypic Age Acceleration/deceleration Prediction Tool for Pets,” and is incorporated herein by reference as if fully set forth.

### BACKGROUND

The disclosure relates to tools to determine phenotypic age of companion animals (a/k/a “pets”, “dogs” or “cats” and used interchangeably herein) and to identify whether such phenotypic age is accelerating or decelerating for an individual companion animal as well as to predict life span. The disclosure also relates to a method for decelerating phenotypic aging of companion animals in need thereof.

Pets are important in the lives of humans. They provide companionship, entertainment and even can improve people's health. However, one of the downsides of pet ownership is that pets do not live as long as people and people feel sadness and grief when they lose their pet. Not all pets, even those of the same initial age and breed, have the same predicted lifespan. In addition, not all pets age at the same rate, with some pets showing evidence that they are aging faster than would be expected based on their chronological age and other pets showing evidence that they are aging slower than would be expected based on their chronological age. Two pets of the same breed and same age may have very different aging trajectories, with one pet having poor health and developing age-related conditions relatively early and another pet having excellent health and developing age-related conditions relatively late or not at all. Poor health may result in a shorter lifespan, but a pet may also live for years with age-related conditions, which may limit the pet's physical abilities, cognitive abilities, care requirements and quality of life while they are living. The impact of the pet's aging trajectory also impacts the pet parent, for example, through increased veterinary and home care requirements, limited ability to perform activities of daily living, medication costs, and the inability to enjoy previously shared activities. Because of the potential impacts of their pet's aging process, pet parents want to take the best care of their pet and ensure their pet remains as healthy as possible for as long as possible. One barrier to selecting the best care possible for their pet is the lack of understanding of the aging process in pets. Pets are quite diverse in their lifespan. This is especially true in dogs where some breeds may have a lifespan of only six years while others can live to twenty years or more. Lifespan of individuals within breeds is also quite variable and is influenced by genetics, healthcare practices, feeding and nutrition, exercise, environment and other factors.

Guidelines for examinations, vaccinations, diagnostic and screening tests and aging-related accommodations are different for senior pets vs. those in earlier lifestages. However, the definition of the senior lifestage in dogs requires identifying dogs that are in the last 25% of their estimated lifespan, a threshold that may be difficult for both pet parents and veterinarians to identify. See Bartges, J. et al. “AAHA Canine Life Stage Guidelines” (2012) JAAHA 48:1. Further dogs are the most phenotypically diverse mammalian species with lifespans that vary widely by breed (Ruple, A et al. “Dog Models of Aging” (2022) Annu. Rev. Anim. Biosci. 10:419-439) in addition to genetic, nutritional and environmental factors.

In cats, the definition of the senior lifestage is a bit more well-defined (cats over 10 years of age are considered senior) See Quimby, J. et al. “2021 AAHA/AAFP Feline Life Stage Guidelines” (2021) JAAHA 57:2. Nevertheless, cat lifespans too can vary widely, with a typical lifespan being reported to be between 13 and 17 years, some cats known to live over 20 years and the oldest cat on record reported to have lived over 38 years. Even in the context of a “typical” lifespan, a pet may spend between 25-40% of its life as a “senior” pet, suggesting that chronological age alone may not be sufficient to reflect the aging process. A better understanding of the aging process in a given pet would allow pet parents and veterinarians to provide more targeted and personalized care.

Some aspects of the aging process may be easier to recognize, such as graying fur, but pets also do not always exhibit easily recognizable changes in appearance or behavior as they age to provide pet parents or veterinarians clues to the pet aging process. Furthermore, even when signs are present, pet parents and veterinarians may not recognize signs of aging in pets, including both dogs and cats, until aging is advanced. Other aspects of the aging process may be invisible without health metrics. Such health metrics would allow pet parents to understand their pet's current phenotypic age (which may be different from the pet's chronological age) and whether their pet is experiencing an accelerated or decelerated rate of aging compared to what would be expected based on chronological age, and based on this information, allow the pet parent to make changes to modifiable factors such as care and nutrition to support the longest, healthiest life possible for their pet. Many common parameters, including measures of body composition (such as the body condition score or body fat index) and/or weight, disease count, clinical chemistry blood measures, markers of nutrition status, inflammation, and organ function, and blood cell counts and characteristics may be used alone or in various combinations with or without chronologic age to derive an index that estimates a pet's phenotypic age (i.e., an aging index based on a range of observed characteristics, rather than a measure calculated using only known date of birth), accelerated/decelerated phenotypic age, patterns in the rate of aging over time (patterns in the difference between phenotypic age and chronological age over time), lifespan, health span, life expectancy, health expectancy, and longevity. In addition to these parameters, measures such as indicators of physical activity and mobility, pain, sleep, behavior, cognition, location/proximity information, temperature, and subjective pet parent information as well as less common parameters (including health markers derived from blood, urine, stool, saliva, skin and mucosa, sweat, tears and tissue including genetic markers, epigenetic markers, chromosomal changes, gene expression markers, endocrine markers, brain health markers, stress markers, immune function and/or inflammation markers, metabolic markers, markers of organ function, neurological parameters, body composition parameters, and/or microbiome parameters such as composition, diversity, function, etc.) can, when used alone or in various combinations with or without more common parameters, indicate aging-related changes far in advance of when they may be recognized by observers such as pet parents and veterinarians.

Relatively simple frameworks for evaluating aging beyond chronological age have been developed. One example of such a framework is the DISHA framework (Landsberg, Nichol and Araujo, Vet Clin Small Anim 42 (2012) 749-768) which may be used by pet parents and veterinarians to evaluate signs of cognitive aging in pets. The DISHA framework refers to signs such as disorientation, a decrease in social interactions, changes in sleep-wake cycles, a loss of prior housetraining, increased anxiety, and changes in level of activity. However, even with such a framework, the assessed characteristics can still be difficult for some pet parents or veterinarians to recognize, observe and/or quantify. For example, pet parents and veterinarians may have difficulty recognizing, observing and/or quantifying sleep-wake cycle alterations or disorientation or changes in social interactions. Additionally, while the DISHA framework provides guidance on behavioral signs of cognitive aging, clear and standardized criteria for diagnosing cognitive dysfunction in pets is lacking. Furthermore, cognitive health is only one component of the aging process in companion animals.

The prior art describes the use of primarily single biomarkers or limited multiple biological inputs in age correlation studies in pets. None of the prior art has utilized larger biological inputs or a combination of clinical, biological, digital, behavioral and phenotypic inputs coupled with machine learning algorithms to predict phenotypic age in pets.

### SUMMARY

While others have evaluated the use of physical activity in predicting age/longevity or death in human populations, an index that incorporates objectively measured aspects of both physical and cognitive aging, alone or in combination with chronological age, for the purpose of quantifying the aging process in dogs and cats is novel and useful. Furthermore, development of one or more panels specific to aging dogs and/or cats (for example, a panel that may include chronological age together with parameters measured in blood, urine, stool saliva, skin and mucosa, sweat, tears and/or tissues, a panel comprised of health parameters that may include physical activity, mobility, sleep, and other behavioral measures, a panel that may include both behavioral and location information, a panel that may include pain measures, cognition measures and other pet parent reported information, etc.) and incorporation of one or more of these panels into an aging index would add information not otherwise observable to pet parents and veterinarians and complement an index based on chronological age alone. The disclosed system may replace or supplement relatively simple frameworks for evaluating age-related changes, such as DISHA for cognitive changes, that are currently in use.

The present disclosure solves the problem of a lack of knowledge about accelerated or decelerated phenotypic age, patterns in the rate of aging over time (patterns in the difference between phenotypic age and chronological age over time), lifespan, healthspan, life expectancy, health expectancy, and longevity, of individual pets by providing a phenotypic age prediction tool for companion animals. Data from wearable devices (e.g., collar-worn accelerometers) in both cats and dogs show behavioral changes in young vs. older animals including differences in the frequency and duration of physical activity, changes in health such as mobility challenges, anxiety related behaviors and patterns of sleep and wake timings, as well as variation between animals of a similar chronological age. As used herein, health span means the amount of time, usually measured in years, a subject is healthy without chronic and debilitating disease.

Younger subjects have been observed to rest more and sleep less, as well as spend more time running and walking compared to older subjects. See, for example, FIG. 1. Based on this, it is possible to include these metrics in a measure of aging. By comparing patterns in behavior data such as running, walking, sleeping, and resting, assessed using a wearable device, across age groups, it has been observed that juniors (aged 18 months or less) differ from adults (18 months—7 years old) in sleeping, walking and running, with juniors exhibiting greater median durations of walking and running and a lower median duration of sleeping compared to adults; adults differ from seniors (>7 years old) in walking, running and sleeping with adults exhibiting greater median durations of walking and running and a lower median duration of sleeping compared to seniors; and juniors differ from seniors in all evaluated metrics with greater median durations of resting, walking and running and a lower median duration of sleeping compared to seniors. This suggests that different life stage categories are characterized by different behavior patterns.

Subjects that were categorized as having mobility issues differed from healthy adult dogs and healthy senior dogs. Dogs with mobility issues differed from dogs without mobility issues in running and walking behavior, exhibiting a smaller range of time spent running, a lower median duration of running behavior, and a lower median duration of walking behavior compared to dogs without mobility issues. See FIG. 2. Dogs with mobility issues differed from healthy adult dogs in walking and running behavior, exhibiting a lower median duration of walking behavior, a lower median duration of running behavior, and a smaller range of running behavior compared to healthy adult dogs. The median durations of walking and running behavior among dogs with mobility issues were similar to those observed among healthy senior dogs, however, healthy seniors showed more variability in the duration of running behavior compared to dogs with mobility issues. Many senior dogs may be suffering from unrecognized and undiagnosed mobility issues, such as osteoarthritis, that may instead be attributed to the natural aging process. See FIG. 3. As such, a comparison between healthy adults, ‘healthy’ seniors and dogs identified as having mobility issues allow the establishment of metrics that may improve recognition of mobility issues in dogs that have yet to be diagnosed. Median behavior durations are very similar between the healthy seniors and dogs with mobility issues for both walking and running, yet healthy seniors showed a much greater range of time spent running compared to dogs with mobility issues, and dogs with mobility issues showed a greater range of time spent walking compared to healthy seniors. Healthy adult dogs showed higher median walking and running durations compared to healthy seniors and healthy adults dogs showed a greater range of walking duration compared to healthy seniors or dogs with mobility issues. Since mobility issues become more common with age, health metrics that can highlight the development of mobility issues early on, before they become clinically significant or start to interfere with daily life, offer the best opportunity to intervene with changes to care and nutrition to support healthy mobility for as long as possible in aging pets. Metrics that can separate groups based on health-related behavior changes that have not yet been recognized by pet parents or diagnosed by veterinarians are an essential tool in this process.

Anxiety is a generalized marker that may be related to characteristics of the aging process, as in the DISHA metrics of disorientation, decreased social interactions, changes in sleep-wake cycles, a loss of prior housetraining, increased anxiety, and changes in their level of activity (Landsberg, Nichol and Araujo, Vet Clin Small Anim 42 (2012) 749-768). The behavior patterns of adult dogs who were categorized as having anxiety but were otherwise healthy were compared to adult dogs who were healthy and categorized as not having anxiety. The anxious dogs appear to sleep slightly more and rest slightly less over the course of a full day (FIGS. 4A and 4b). Further analysis of the timings of sleep behaviors show that the majority of this difference is expressed during the nighttime, particularly for resting behavior, (FIGS. 5A & 5B), as daytime sleep and rest rates are much closer (FIGS. 6A & 6B).

In an aspect, the disclosure relates to a system for generating a multi-component aging index for an individual companion animal based on measuring at least one of digital biomarkers, traditional biomarkers and subjective assessment methods to predict phenotypic age and phenotypic age acceleration/deceleration in dogs and cats. The system optionally further comprises determining at least one of sex, neuter status, and lifestage.

In some embodiments, the biomarker panels comprise two or more traditional biomarkers selected from CBC/chemistry parameters, fecal microbiome, fecal metabolites, urinary microbiome, urinary metabolites, blood metabolites, and blood biomarkers comprising albumin, creatinine, glucose, lymphocyte percent, mean cell volume, red cell distribution width, alkaline phosphatase, white blood cell count, SDMA, circulating peptides comprising Aβ42, circulating postbiotics, immunoglobulins, immunoglobulin M, growth hormone (GH)/insulin growth factor-1 (IGF-1), and DNA biomarkers, SNPs, and genetic variants.

In some embodiments, the system further comprises the system further comprising at least one of wearable devices. In some embodiments, the wearable devices measure physical activity. In some embodiments, the physical activity measured comprises at least one of walking, running, resting, jumping, sleep time, sleep quality and sleep regularity.

In some embodiments, the system further comprises subjective assessment. In some embodiments, the subjective assessments are via at least one of pet parent questionnaires and veterinary questionnaires.

In some embodiments, the system further comprises clinical characteristics. In some embodiments, the clinical characteristics comprise at least one of chronological age, weight, BCS, BFI, temperature, respiration rate, and heart rate.

In some embodiments, the system further comprises one or more environmental sensor. In some embodiments, the one or more environmental sensor comprises one or more sensor detecting at least one of location, location-based behaviors, and activities. In some embodiments, the activities comprise at least one of proximity to pet parent, play, timing and frequency of feeding, location of eating, drinking, urination and defecation, body posture, pose estimation, tail position, body position, and movement tracking over time.

In some embodiments, the system further comprises repeated measures of at least one of clinical, digital and biological data.

In some embodiments, the system further comprises at least one of eating, drinking, urinating, defecating patterns.

In some embodiments, the system further comprises signs of at least one of emotional health, cognitive health, fear, anxiety, stress, dementia, social interaction with humans and interactions with other animals.

In some embodiments, the system further comprises veterinary assessment. In some embodiments, the veterinary assessment comprises at least one of gastrointestinal disease, genitourinary disease, kidney disease, dermatological disease, respiratory disease, neurological disease, muscular disease, ophthalmological disease, auditory disease, cardiovascular disease, cancer, oral health, endocrine disease, infectious disease, immune function, inflammation, orthopedic disease, mobility, and pain.

In an aspect, the disclosure relates to a method for decelerating phenotypic aging of a companion animal in need thereof comprising determining an index using a system or method herein and based thereon providing at least one of customized health, dietary or nutrition measures to the companion animal.

In an aspect the disclosure provides a system for generating a multi-component aging index for an individual companion animal comprising digital biomarkers, traditional (biological) biomarkers, a range of health parameters and subjective assessment methods to predict phenotypic age (i.e., an aging index based on a range of observed characteristics, rather than a measure calculated using only known date of birth), accelerated/decelerated phenotypic age, patterns in the rate of aging over time (patterns in the difference between phenotypic age and chronological age over time), lifespan, healthspan, life expectancy, health expectancy, and longevity in dogs and cats. The multi-component index includes, without limitation, measurements from wearable devices regarding specific forms of physical activity and/or mobility and/or behaviors measured alone or in combination including but not limited to walking, running, resting, jumping, scratching, shaking, licking, chewing, sniffing or ground-tracking, exploration, tail wagging, play bowing, panting, circling, climbing (e.g., stair climbing), digging, vocalizing, abnormal gait, pacing, eating, drinking, urinating, defecating, vomiting, pain, lameness, reproductive and/or mating behavior, seizures, sleep health (including but not limited to sleep duration, sleep timing, sleep duration variability, sleep quality, sleep quality variability and the regularity of sleep timing), subjective assessment methods such as pet parent questionnaires and veterinarian questionnaires, and clinical characteristics, including, without limitation chronological age, body composition (such as the body condition score, body fat index or objectively measured body composition parameters), body weight, temperature, respiration, heart rate, pulse, sex, neuter status, lifestage, and disease count.

In some embodiments, the index further utilizes environmental sensors to assess location, proximity and thus location/proximity-based behaviors and activities, including, without limitation signs of health such as intake and appropriate or inappropriate output behaviors (such as occurrence of eating, drinking, urinating, defecating, vomiting, hairballs and the like), signs of illness such as gastrointestinal disease, genitourinary and/or kidney disease, dermatological disease, respiratory disease, neurological disease, muscular disease, ophthalmological disease, auditory disease, cardiovascular disease, cancer, oral health, endocrine disease, infectious disease, immune function and/or inflammation and orthopedic disease and/or mobility/pain, and signs of emotional/cognitive health, including but not limited to fear, anxiety, stress, dementia, cognitive function, attention, and social interaction with humans and other animals.

In some embodiments, the index further utilizes blood, urine, stool, saliva, skin and mucosa, sweat, tears, tissue or other health parameters alone or in combination ex., panels including, without limitation, clinical chemistry blood measures, markers of nutrition status, inflammation, and organ function, and blood cell counts, genetic markers, epigenetic markers, chromosomal changes, gene expression markers, endocrine markers, brain health markers, stress markers, immune function and/or inflammation markers, metabolic markers, markers of organ function, neurological parameters, and/or microbiome composition/diversity/function measures. Such parameters could include, but are not limited to albumin, creatinine, glucose, lymphocyte percent, mean cell volume, red cell distribution width, alkaline phosphatase, white blood cell count, SDMA, circulating peptides such as Aβ42, circulating postbiotics, immunoglobulins such as immunoglobulin M, growth hormone (GH)/insulin growth factor-1 (IGF-1), and DNA biomarkers including SNPs). In some embodiments, the index further utilizes a DNA methylome.

In some embodiments, the index further incorporates data over a period of months or years, versus just a one-time sample. Such data includes, without limitation, the use of an expanded set of environmental sensors to observe proximity to pet parent, play, timing and frequency of feeding, location of eating, drinking, urination and defecation, body posture (e.g., via pose estimation), tail position, body position, movement tracking over time, and incorporation of repeated measures of selected clinical, digital and biological data. This provides for a phenotypic age index that is continuously updated as the pet is providing continuous data streamed through connected health devices as part of a connected health IoT ecosystem. Such devices include, without limitation, cameras, microphones, eye-tracking devices, touchscreens, smart litterboxes, smart pet beds, smart scales, smart feeders, smart collars or harnesses, smart clothing, smart thermometers, smart toys, smart water fountains, smart pet doors, in addition to one or a plurality of wearable sensors with accelerometer/gyroscope/magnetometer/thermometer/light sensors/Wi-Fi and Bluetooth capabilities and proximity beacons. Machine learning algorithms are implemented over the data recorded by smart devices (including the wearable sensor or other smart devices), in ancillary technologies via distributed computing topology (“edge computing”), or in the cloud.

The present disclosure therefore relates to methods of identifying an interaction between a metric and a likelihood that the pet or subject is healthy or unhealthy, the method comprising: (a) quantifying one or a plurality of metrics of a pet over a time period; (b) calculating a score corresponding to a quantity relative to a control subject; and (c) correlating the score with the likelihood that subject is unhealthy or healthy depending upon the quantity of the one or plurality of metrics.

The disclosure further relates to methods of identifying an age of a subject, the method comprising: (a) quantifying one or a plurality of metrics; (b) calculating a score corresponding to the one or plurality of metrics from the subject; and (c) correlating the score with the likelihood that the subject has a certain age (e.g., phenotypic age).

The disclosure further relates to methods of identifying a subject likely to respond to a age-related disorder treatment, the method comprising: (a) calculating a score corresponding with one or a plurality of metrics; and (b) correlating the score with a likelihood that a subject has a statistically relevant disorder, wherein if the score is above a first threshold, then the subject is likely to respond to an age-related disorder treatment, and wherein if the score is below the first threshold, then the subject is not likely to respond to the disorder treatment.

The disclosure further relates to methods of identifying a subject likely to respond to a nutrition intervention to mitigate the effects of aging, the method comprising: (a) calculating a score corresponding with one or a plurality of metrics; and (b) correlating the score with the presence of age-related changes, wherein if the score is above a first threshold, then the subject is likely to respond to a nutritional intervention, and wherein if the score is below the first threshold, then the subject is not likely to respond to the nutritional intervention.

The disclosure further relates to methods of predicting a likelihood that a subject does or does not respond to an age-related disorder treatment, the method comprising: (a) compiling a quantity of metrics from a population that has presented one or a plurality of metrics, wherein the population includes the subject; (b) calculating the quantity or frequency of a metric by sensing one or plurality of metrics associated with an age-related disorder to predict an age or behavior associated with the age-related disorder; (c) calculating a score; (d) correlating the score with the likelihood that the subject has a disorder based upon the metric; and (e) selecting a treatment or intervention for the subject based upon the metric or score.

The disclosure further relates to methods of predicting a likelihood that a subject does or does not respond to a nutrition intervention to mitigate the effects of aging, the method comprising: (a) compiling a quantity of metrics from a population that has presented one or a plurality of metrics, wherein the population includes the subject; (b) calculating the quantity or frequency of a metric by sensing one or plurality of metrics associated with a disorder to predict an age or behavior associated with the disorder; (c) calculating a score; (d) correlating the score with the presence of age-related changes based upon the metric; and (e) selecting a treatment for the subject based upon the metric or score.

The disclosure further relates to computer software/program products encoded on a computer-readable storage medium, wherein the computer program product comprises instructions for: (a) identifying a metric associated with age of the subject; and (b) calculating a score quantifying the metric.

The disclosure further relates to systems for predicting an age (e.g., phenotypic age, lifespan, healthspan, or aging acceleration/deceleration) of a subject, the system comprising: (a) a processor operable to execute programs; (b) a memory associated with the processor; (c) a database associated with said processor and said memory; and (d) a program stored in the memory and executable by the processor, the program being operable for: (i) performing analysis on subject with a quantity of a metric associated with a disorder; (ii) identifying dysfunctional metric quantity associated with the disorder; and (iii) calculating a score corresponding to the metric.

In an aspect, the disclosure relates to a system comprising a biosensor, at least one computer storage memory, and a controller. In some embodiments, the biosensor comprises at least one or all of a solid support comprising an internal cavity and external surface, a band operably linked to an external surface of the solid support, and an electrical circuit positioned within the internal cavity. In some embodiments, the electrical circuit comprises at least one of a first position sensor and a first motion sensor. In some embodiments, each of the sensors is in electrical communication with the controller.

In an aspect, the disclosure relates to a method of determining acceleration or deceleration of age of a subject. The method comprises measuring one or a combination of activity metrics of the subject over a period of time; determining a mobility score of the subject relative to a control subject of the same age; classifying the subject as active if the mobility score is at or over the control mobility score for the age of the subject; or classifying the subject as inactive if the mobility score is under the control mobility score for the age of the subject.

In an aspect, the disclosure relates to a method of determining the phenotypic age of a subject, The method comprises measuring one or a combination of activity metrics of the subject over a period of time; determining a mobility score of the subject relative to a control subject of the same age; classifying the subject as healthy if the mobility score is at or over the control mobility score for the age of the subject; or classifying the subject as unhealthy if the mobility score is under the control mobility score for the age of the subject; and/or (d) determining the age of the subject based upon the mobility score.

In an aspect, the disclosure relates to a computer program product encoded on a computer-readable storage medium. The computer program product comprises instructions for: receiving data from one or a plurality of biosensors on a subject; calculating a mobility score based upon the data; and determining the level of activity or age of a subject based upon the mobility score.

In an aspect, the disclosure relates to a biosensor. The biosensor comprises a top and a bottom exterior surface separated by a height. The exterior surface and the height define an internal cavity comprising at least one sensor. In some embodiments, the at least one sensor is at least one of: a gyroscope, at least a first pressure sensor, at least a first temperature sensor, and at least a first accelerometer. In some embodiments, the biosensor further comprises a controller. In some embodiments, each of the at least one sensor are in electrical communication with a controller by way of an electrical circuit.

In an aspect, the disclosure relates to a smart bed. In some embodiments, the smart bed comprises a frame, a base, and sensory equipment.

In an aspect, the disclosure relates to a smart room. In some embodiments, the smart room comprises at least one of (1) one or more biosensor herein and (2) one or more smart bed herein.

### BRIEF DESCRIPTION OF THE DRAWINGS

FIG. 1 shows box plots showing comparison of activity-related behaviors between healthy dogs in each age group. Box plots show median, IQR, minima, maxima and outliers represented by red crosses.

FIG. 2 shows box plots showing comparison of forward motion behaviors between dogs diagnosed with mobility challenges and those not. Box plots shows median, IQR, minima, maxima, and outliers.

FIG. 3 shows box plots showing comparison of forward motion behaviors between dogs diagnosed with mobility challenges, senior dogs not diagnosed with mobility and healthy adults.

FIG. 4 shows box plots showing comparison between anxious subjects and non-anxious subjects sleep and rest rates over 24 hours.

FIG. 5 shows box plots showing comparison between anxious subjects and non-anxious subjects sleep and rest rates throughout the night (00:00-06:00).

FIG. 6 shows box plots showing comparison between anxious subjects and non-anxious subjects sleep and rest rates throughout the day (06:00-00:00).

FIG. 7 shows an example of a wearable sensor in the form of a lightweight digital tracker worn on the collar.

FIG. 8 shows how a wearable device captures movement in three dimensions.

FIG. 9 shows how 3D motion is translated into behaviors.

FIGS. 10A-10E show components and activity flow chart of a Collar Mounted Activity Sensor (CMAS). FIG. 10A shows an exterior view of a CMAS. FIG. 10B shows an interior view of the CMAS. FIG. 10C shows one view of a printed circuit board in the CMAS. FIG. 10D shows a second view of a printed circuit board in the CMAS. FIG. 10E shows a flow diagram of information within the CMAS.

FIGS. 11A-D show various views of an updated CMAS utilizing HPN1 technology. HPNI technology includes a data collection rate for the accelerometer of 104 Hz, or optionally 50 Hz or 100 Hz. Also optionally, the HPN1 technology includes a configurable data collection rate. The sensors having HPN1 technology may also include a gyroscope and/or one or more other specific sensing devices; e.g., others listed herein.

FIGS. 12A and 12B show a new metal collar design that overcomes issues with nylon collar destruction and fitting.

FIGS. 13A and 13B show signal magnitude data from a wearable sensor. FIG. 13A shows a pattern for scratching. FIG. 13B shows a pattern for running.

FIG. 14 shows how health states may be characterized by multivariate behavior changes.

FIG. 15 shows an example of the use of computer vision to quantify behaviors in groups of pets.

FIGS. 16A and 16B show how computer vision can reduce the complex geometry of a detected individual to a simpler shape, such as an ellipse, allowing orientation of the dogs with respect to the environment, to an object and/or to each other.

FIGS. 17A-17D show representations of audio data which can characterize vocalizations to quantify animal behavior type, timing, duration, or intensity, to quantify the number and type of animals participating in a behavior, and to provide context to other measurements such as postures, behaviors and/or interactions captured by a wearable device and/or computer vision.

FIGS. 18A and 18B show how computer vision can focus on a target region on the pet and look for changes.

FIGS. 19A-19D smart bed embodiments. FIGS. 19A and 19B show examples of a pet smart bed. FIG. 19C shows a more detailed diagram of an exemplary smart bed. FIG. 19D shows an example of a smart room.

FIGS. 20A and 20B show diagrams of exemplary smart toys used to generate data for the index.

FIG. 21 shows changes in activity in cats by age.

FIG. 22 shows a daily activity profile for an “average” cat.

FIG. 23 compares daily activity profiles for all cats regardless of age or health state, healthy adult cats and arthritic adult cats.

FIG. 24 shows daily activity of a healthy adult cat.

FIG. 25 shows daily activity of a senior cat with past health issues.

FIG. 26 shows daily activity of a five-month old kitten.

FIG. 27 shows cat accelerometers in use.

FIG. 28 shows a smart system set up for cats.

FIGS. 29A and 29B depict session metrics for Aging and Non-aging.

FIG. 30A through 30J depict sessionization data of groups of pets.

FIG. 31 depicts a study design associated with a dog experiencing a diet change.

FIG. 32 depicts a set of study design endpoints.

FIG. 33 depicts a set of metrics measured on a wearable sensor.

FIG. 34 depicts a methods summary of a study design including a wearable sensor on a subject.

FIG. 35A through 35F depict a set of data related to activity measurements of pets.

FIG. 36A through 36X depict embodiments of a wearable sensor incorporated in a collar for a domesticated animal.

FIG. 37 depicts a modified embodiments for a wearable sensor incorporated in a collar for a pet with an HPN1.

FIG. 38 depicts a design of a sensor incorporated in a bedding of a pet.

FIG. 39A-39E depict embodiments of the sensor incorporated within bedding for a pet.

FIGS. 40A and 40B depict a collection of metrics to be measured with embodiments of the sensor incorporated within bedding for a pet.

FIGS. 41A and 41B depict an electrical circuit of the sensor embedded in the collar device or the embodiments of the sensor incorporated within bedding for a pet.

FIG. 42 depicts the metric detection system components in connection with a web-enabled device.

FIG. 43 depicts an image of a web-based application and computer program product displaying one or a plurality of metrics.

FIG. 44 illustrates overall survival among felines. As noted in Table 11a, 109 of 721 (15.1%) of felines died during the 3-year follow-up; Kaplan-Meier curves for felines overall.

FIGS. 45A, 45B, and 45C illustrate Kaplan-Meier curves for felines in the highest 20% versus the lowest 20% of PhenoAgeAccel (Model 1).

FIGS. 46A, 46B, and 46C illustrate Kaplan-Meier curves for felines in the highest 20% versus the lowest 20% of PhenoAgeAccel (Model 2).

FIGS. 47A, 47B, and 47C illustrate Kaplan-Meier curves for felines in the highest 20% versus the lowest 20% of PhenoAgeAccel (Model 3).

FIGS. 48A and 48B illustrate the relationship between Phenotypic Age, chronological age, and PhenoAgeAccel (Model 1) in felines. FIG. 48A shows the correlation between phenotypic age and chronological age/FIG. 48B shows PhenoAgeAccel distribution.

FIGS. 49A and 49B illustrate the relationship between Phenotypic Age, chronological age, and PhenoAgeAccel (Model 2) in felines. FIG. 49A shows the correlation between phenotypic age and chronological age. FIG. 49B shows the PhenoAgeAccel distribution.

FIGS. 50A and 50B illustrated the relationship between Phenotypic Age, chronological age, and PhenoAgeAccel (Model 3) in felines. FIG. 50A shows the correlation between phenotypic age and chronological age. FIG. 50B shows the PhenoAgeAccel distribution.

FIG. 51 illustrates receiver operating characteristic curves for 2-year mortality by model in felines.

FIG. 52 illustrates overall survival among canines. As noted in Table 11b, 89 of 709 (12.6%) of canines died during the 3-year follow-up; Kaplan-Meier curves for canines overall.

FIGS. 53A, 53B, and 53C illustrate Kaplan-Meier curves for canines in the highest 20% versus the lowest 20% of PhenoAgeAccel (Model 1).

FIGS. 54A, 54B, and 54C illustrate Kaplan-Meier curves for canines in the highest 20% versus the lowest 20% of PhenoAgeAccel (Model 2).

FIGS. 55A, 55B, and 55C illustrate Kaplan-Meier curves for canines in the highest 20% versus the lowest 20% of PhenoAgeAccel (Model 3).

FIGS. 56A and 56B illustrate the relationship between Phenotypic Age, chronological age, and PhenoAgeAccel (Model 1) in canines. FIG. 56A shows the correlation between phenotypic age and chronological age. FIG. 56B shows the PhenoAgeAccel distribution.

FIGS. 57A and 57B illustrate the relationship between Phenotypic Age, chronological age, and PhenoAgeAccel (Model 2) in canines. FIG. 57A shows the correlation between phenotypic age and chronological age. FIG. 57B shows the PhenoAgeAccel distribution.

FIGS. 58A and 58B illustrate the relationship between Phenotypic Age, chronological age, and PhenoAgeAccel (Model 3) in canines. FIG. 58A shows the correlation between phenotypic age and chronological age. FIG. 58B shows the PhenoAgeAccel distribution.

FIG. 59 illustrates receiver operating characteristic curves for 2-year mortality by model in canines.

### DETAILED DESCRIPTION

For illustrative purposes, the principles of the present disclosure are described by referencing various exemplary embodiments thereof. Although certain embodiments of the disclosure are specifically described herein, one of ordinary skill in the art will readily recognize that the same principles are equally applicable to and can be employed in other compositions and methods. Before explaining the disclosed embodiments of the present disclosure in detail, it is to be understood that the disclosure is not necessarily limited in its application to the details of any particular embodiment disclosed. The terminology used herein is for the purpose of description and not of limitation.

As used herein and in the appended claims, the singular forms “a”, “an”, and “the” include plural references unless the context dictates otherwise. The singular form of any class of the ingredients refers not only to one ingredient within that class, but also to a mixture of those ingredients. The terms “a” (or “an”), “one or more” and “at least one” may be used interchangeably herein. The terms “comprising”, “including”, and “having” may be used interchangeably. The term “include” should be interpreted as “include but are not limited to”. The term “including” should be interpreted as “including but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc. The term “about” when referring to a number means any number within a range of 15% of the number.

The abbreviations and symbols as used herein, unless indicated otherwise, take their ordinary meaning. The abbreviation “wt. %” means percent by weight with respect to the pet food composition. The symbol “°” refers to a degree, such as a temperature degree or a degree of an angle. The symbols “h”, “min”, “mL”,” nm”, “μm” means hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

As used herein, “substantially equal” means within a range known to be correlated to an abnormal or normal range at a given measured in some embodiments, “substantially equal” means that the associated term is from about +/−10% of where an equal value would be associated with whatever metric is modified by the term. For example, if a control sample is from a diseased patient, substantially equal is, in some embodiments, within an abnormal range +/−10% of the abnormal value. If a control sample is from a patient known not to have the condition being tested, substantially equal is within a normal range for that given metric. In some embodiments, the probability of having an age-related disorder by any of the methods disclosed herein is from about 1.01 to about 2.00 times the probability of having an age-related disorder if the subject were a control subject.

As used herein, the term “subject,” “individual” or “patient,” used interchangeably, means any animal, including mammals, such as mice, rats, other rodents, rabbits, dogs, cats, swine, cattle, sheep, horses, or primates. The term “subject” is used, in some embodiments, throughout the specification to describe an animal from which a sample is taken. In some embodiment, the subject is a domesticated pet. For analysis of those conditions which are specific for a specific subject, such as a dog, the term “patient” may be interchangeably used. In some instances in the description of the present disclosure, the term “patient” will refer to humans inactive or active pets, or healthy or unhealthy pets. In some embodiments, the subject may be a dog suspected of having aging-related disorder or being identified as at risk to develop a type age-related disorder. In some embodiments, the subject may be diagnosed as having at resistance to one or a plurality of treatments to treat a disease or disorder afflicting the subject. In some embodiments, the subject may be a non-human animal from which metrics are obtained.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the terms “treat,” “treated,” or “treating” can refer to therapeutic treatment and/or prophylactic or preventative measures wherein the object is to prevent or slow down (lessen) an undesired physiological condition, disorder or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment can also include eliciting a clinically significant response without excessive levels of side effects. Treatment also includes prolonging survival of a pet as compared to expected survival pet if not receiving treatment.

As used herein, the terms “diagnose,” “diagnosing,” or variants thereof refer to identifying the nature of a physiological condition, disorder or disease. In some embodiments, diagnosing a subject refers to identifying whether a subject has an age-related disorder. In some embodiments, diagnosing refers to distinguishing between an unhealthy or healthy pet or subject.

As used herein, “control sample” or “reference sample” refer to samples with a known presence, absence, or quantity of metric being measured, that is used for comparison against an experimental sample.

A “score” is a numerical value that may be assigned or generated after normalization of the value based upon the presence, absence, or quantity of substrates or enzymes disclosed herein. In some embodiments, the score is normalized in respect to a control raw data value.

All references cited herein are hereby incorporated by reference in their entireties. In the event of a conflict in a definition in the present disclosure and that of a cited reference, the present disclosure controls.

Detection of aging signs before they are clinically significant offers opportunities for interventions, such as nutrition or lifestyle modifications, that have the potential to alter the aging trajectory; therefore, early detection through non-invasive means such as monitoring either alone or in combination, measures of the physical body such as body weight, indicators of body composition such as the body condition score or body fat index, as well as other health parameters, including physical activity and mobility, sleep parameters, behavioral parameters such as the frequency or duration of behaviors, pain measures, cognition measures, location and/or proximity information such as the frequency and duration of presence in a particular area, temperature, and subjective information reported by pet parents and/or veterinarians offers opportunities for identifying age-related changes. These signs can be enhanced through the use of health markers derived from blood, urine, stool, saliva, skin and mucosa, sweat, tears and tissue and other health parameters (ex., clinical chemistry parameters, nutrition status markers, blood cell counts, genetic markers, chromosomal changes, gene expression markers, endocrine markers, brain health markers, stress markers, immune function and/or inflammation markers, metabolic markers, markers of organ function (for example, liver, heart, lungs, etc.), neurological parameters, body composition parameters, and/or microbiome parameters such as composition, diversity, function, etc.) that offer more in-depth information about the type of aging-related changes and the systems impacted and enable characterization of the degree of aging-related changes (acceleration or deceleration). Machine learning approaches can be used to develop models able to identify patterns in behavior and activity as well as in digital biomarkers and traditional biomarkers, clinical parameters and subjective information reported by pet parents and/or veterinarians that are indicative of aging in dogs and cats, which can further be applied to predict phenotypic age, accelerated/decelerated phenotypic age, patterns in the rate of aging over time (patterns in the difference between phenotypic age and chronological age over time), lifespan, health span, life expectancy, health expectancy, and longevity in new groups of dogs and cats. Digital biomarkers include data gathered by a sensor herein. Non-limiting examples of digital biomarkers include wearables data (for example, total activity, walking time and/or day, running time and/or day, sleep time and/or day, sleep quality, scratching time and/or day, shaking time and/or day, duration of activity episodes (walking, running, scratching, shaking, sleeping), as well as eating, drinking, urinating and defecating as well as location and proximity data. Further non-limiting examples of digital biomarkers include measures of memory and learning such as from digital toys and/or video games for pets (for example, PupPod). These could include measures of at least one of associative learning, learning speed, task switching, or attention. Still further non-limiting examples of digital biomarkers include weight, heart rate, respiration rate, temperature from a wearable device or a smart pet bed. In some embodiments, the digital biomarkers include at least one of running time and/or day, longest duration of uninterrupted running, walking time and/or day, longest duration of uninterrupted walking, stride length, walking speed, sleep duration, consistency in sleep duration, sleep timing, consistency in sleep timing, sleep quality and consistency in sleep quality. In some embodiments, the digital biomarkers include all of running time and/or day, longest duration of uninterrupted running, walking time and/or day, longest duration of uninterrupted walking, stride length, walking speed, sleep duration, consistency in sleep duration, sleep timing, consistency in sleep timing, sleep quality and consistency in sleep quality.

Once pet parents and veterinarians have information about where an individual pet falls on a lifespan timeline, how their pet's phenotypic age today compares to the pet's chronological age, as well as whether the current rate of aging is faster or slower than would otherwise be expected, and could observe patterns in the rate of aging over time, the pet parents and veterinarians are then able to optimize the care given to the pet and make decisions that could impact overall lifespan, aging trajectory and/or quality of life for the pet.

The disclosure relates to tools and system to determine phenotypic age of companion animals and to identify whether the phenotypic age in a given pet differs from chronological age and thus whether aging is accelerated or decelerated in the pet. The disclosure relates to tools and system to determine whether the lifespan or healthspan of a given pet differs from what otherwise might be expected. The disclosure also relates to a method for decelerating phenotypic aging of companion animals in need thereof. The present disclosure solves the problem of a lack of knowledge about lifespan, life expectancy, healthspan, health expectancy, longevity and rate of aging of an individual pet by providing a phenotypic age prediction tool for companion pets. Wearables data in both cats and dogs shows behavioral changes in young vs. older animals including differences in the amount and timing of physical activity, changes in disease states such as mobility issues, behaviors such as anxiety and the circadian rhythms or patterns of sleep and awake time. In some embodiments, metrics of disclosure relate to lifespan, life expectancy, healthspan, health expectancy, longevity and rate of aging of an individual subject or group of subjects.

The disclosure relates to a system, composition, and series of methods of using the systems and compositions for the analysis of a sample from a subject to accurately diagnose, prognose, or classify the subject as aging or unhealthy due to age. The disclosure also relates to a system, composition, and series of methods of using the systems and compositions for the analysis of pet activities or behavior from a subject to accurately diagnose, prognose, or classify the subject as aging or having a certain phenotype, age, or life expectancy as compared to a control subject or control population. In some embodiments, the system of the present invention comprises a means of detecting and/or quantifying or observing data comprising the frequency of a metric, behavior or activity a subject or pet; and correlating that data with a subject's medical history to predict clinical outcome, treatment plans, preventive medicine plans, or effective therapies.

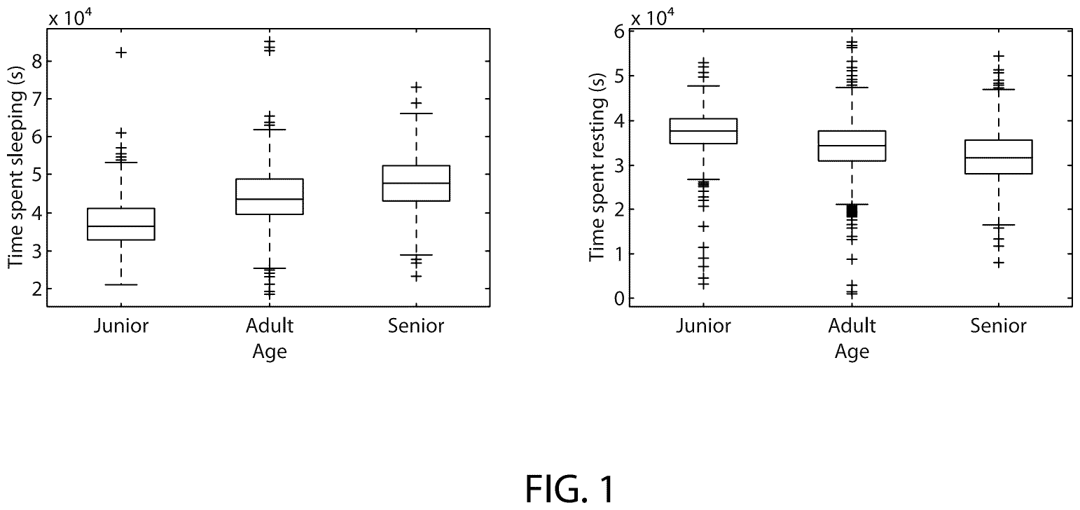

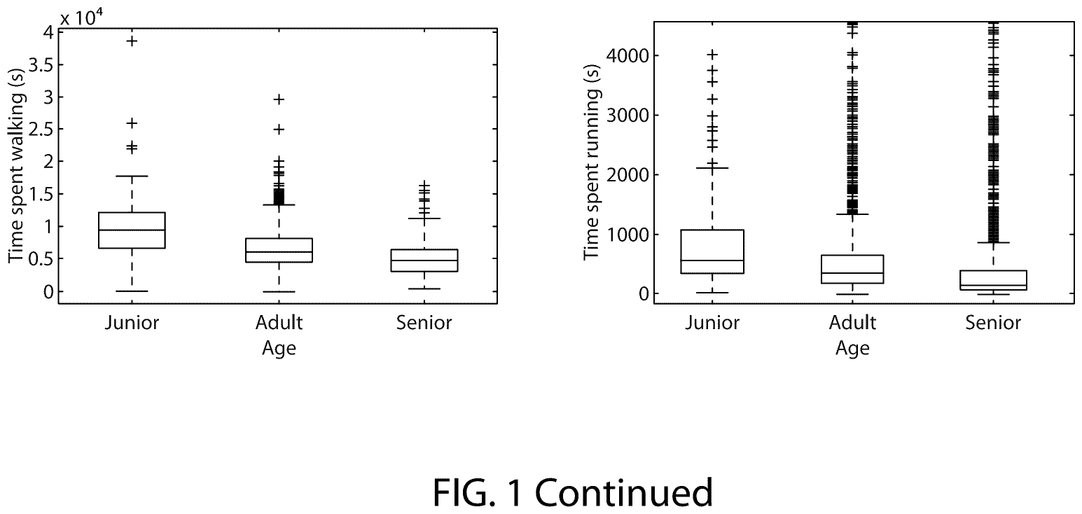

As shown in FIG. 1, younger subjects tend to rest more and sleep less, as well as spending more time running and walking. Based on this, it is possible to include these metrics in a measure of aging, given a demonstration of this trend through an observational study of canine behavior. As shown in Table A, show that juniors differ significantly from adults in sleep, walking and resting; adults differ significantly from seniors in walking, running, sleeping and resting; and juniors differ significantly from seniors in all measured metrics.

TABLE A
Index data for sleep, rest and walking

| | p-value | | |
|---|---|---|---|
| | Junior-Adults | Adults-Seniors | Juniors-Seniors |
| Sleep | 0.004 | 0.092 | <0.001 |
| Rest | 0.094 | 0.333 | 0.011 |
| Walk | 0.011 | 0.007 | <0.001 |

Given that these behaviors appear to change as the subject ages, it is expected to see a reflection of that in observations made at a higher temporal resolution. By visualizing daily median values, it is expected to see these changes become apparent over a long enough period of time.

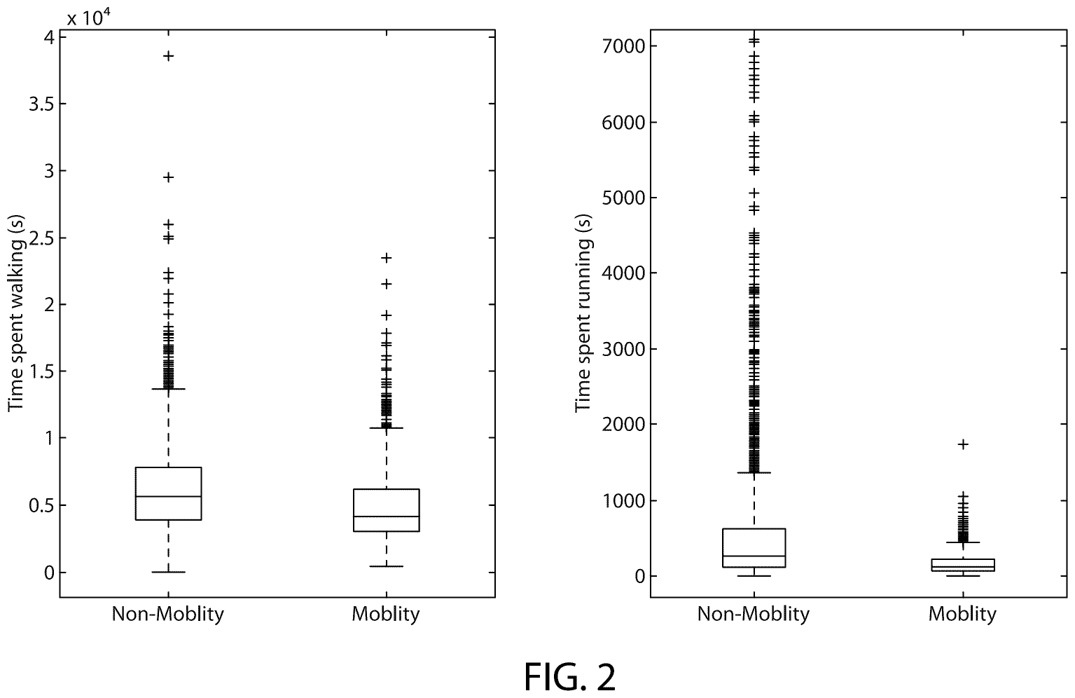

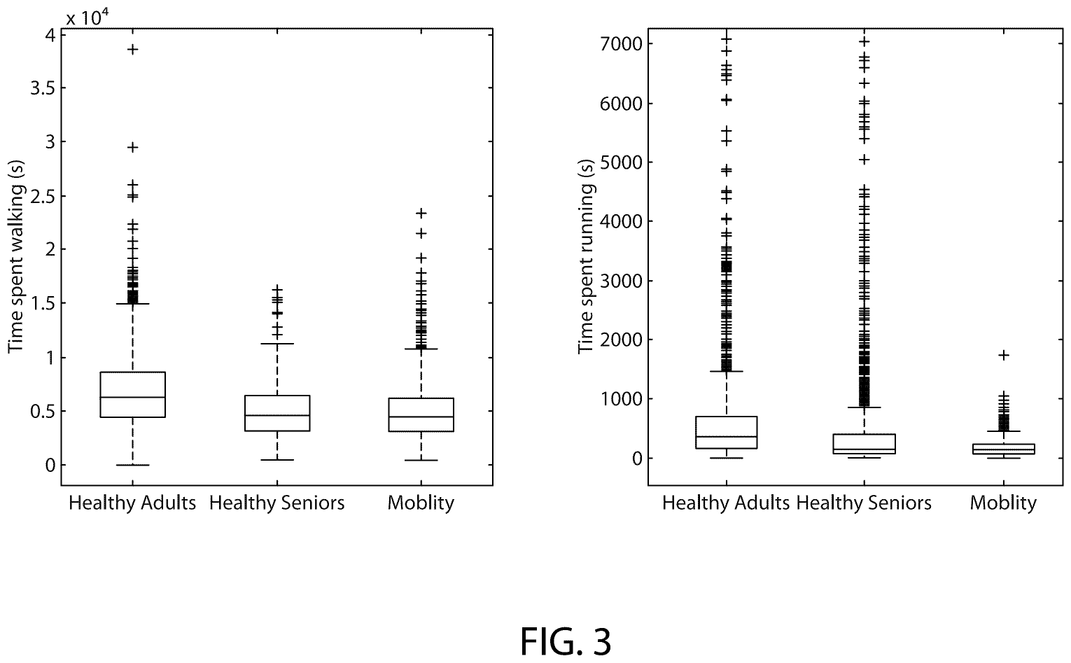

As shown in FIG. 2, there is a slight reduction in the median amount of time spent walking and running for dogs with mobility issues; however, it is possible that some dogs categorized as healthy senior dogs may be suffering from undiagnosed mobility issues that are instead attributed to old age, which could make the difference between the groups appear artificially small. Many senior dogs suffer from mobility challenges such as arthritis while not being diagnosed. As such a comparison between healthy adults (n=34), ‘healthy’ seniors (n=30) and dogs that have been diagnosed with mobility challenges (n=10) allows the development of metrics to identify dogs that have yet to be diagnosed. As shown in FIG. 3, median exhibition rates are very similar between the healthy seniors and dogs with mobility issues for both walking and running though the pattern of behavior expression is different between the two populations, particularly for running, with dogs with mobility issues showing a substantially smaller range of running duration compared to healthy seniors. Dogs with mobility issues show lower median running duration and a substantially smaller range of running duration compared to healthy adults. Dogs with mobility issues also show a lower median duration of daily walking behavior and a somewhat smaller range of daily walking duration compared to healthy adult dogs. These findings suggest that changes in the median daily duration and/or range of daily durations for activity and/or forward motion behaviors such as walking and/or running may be indicators of mobility issues in dogs, helping to differentiate them from healthy dogs. Running behavior may be a particularly sensitive indicator of mobility issues in dogs, and the median daily running duration and/or the range of running durations in groups of dogs may be able to differentiate dogs with mobility issues from healthy seniors and/or healthy adults.

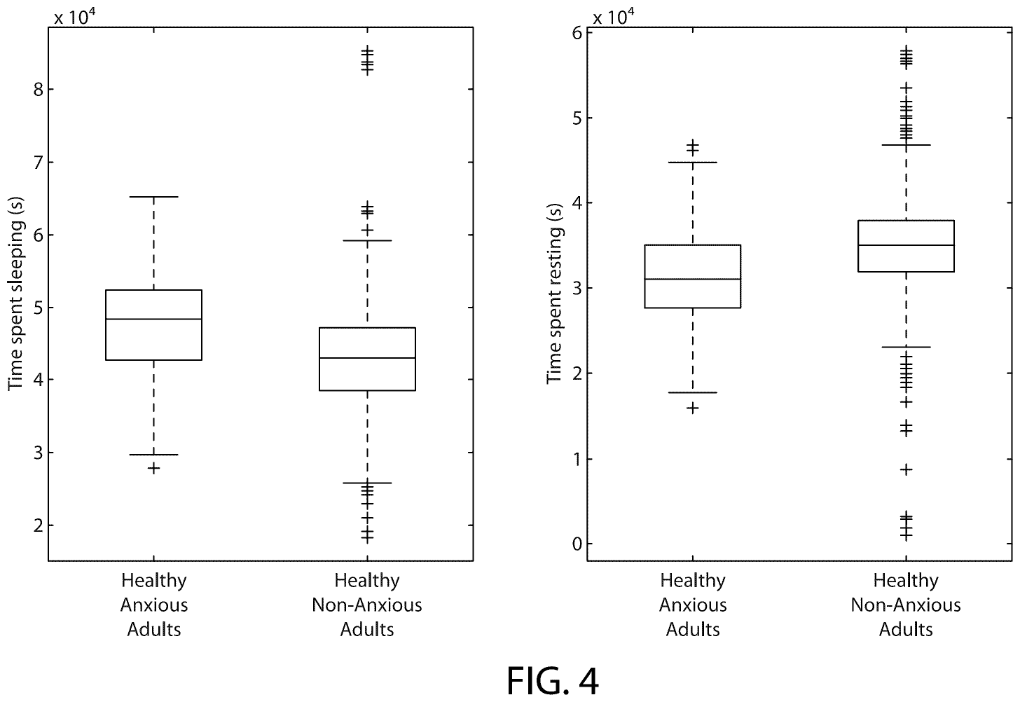

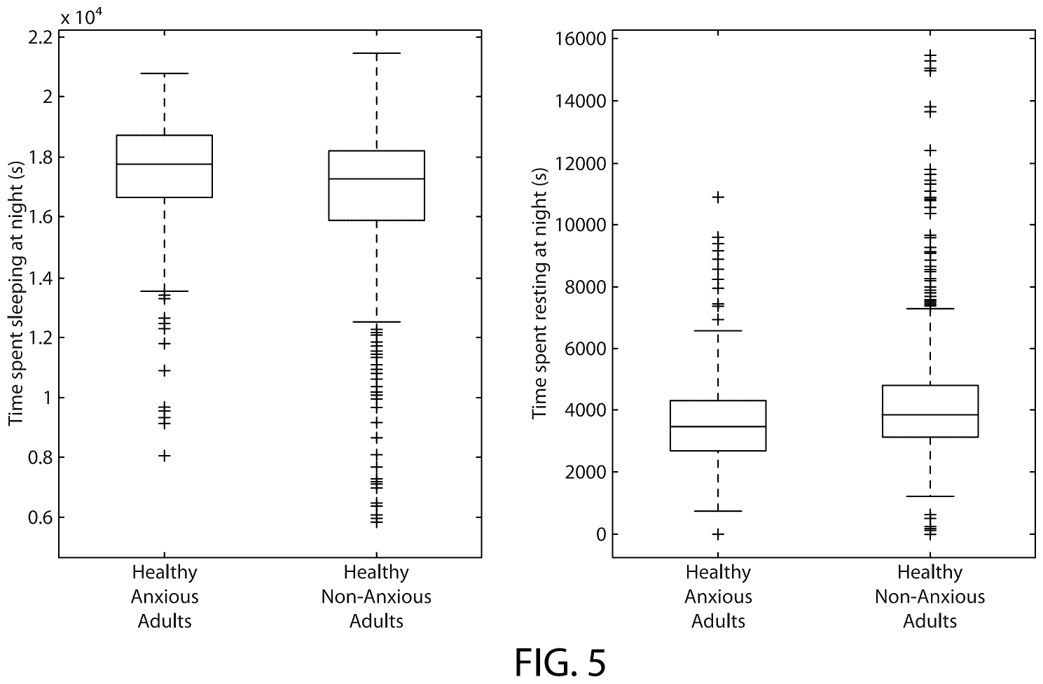

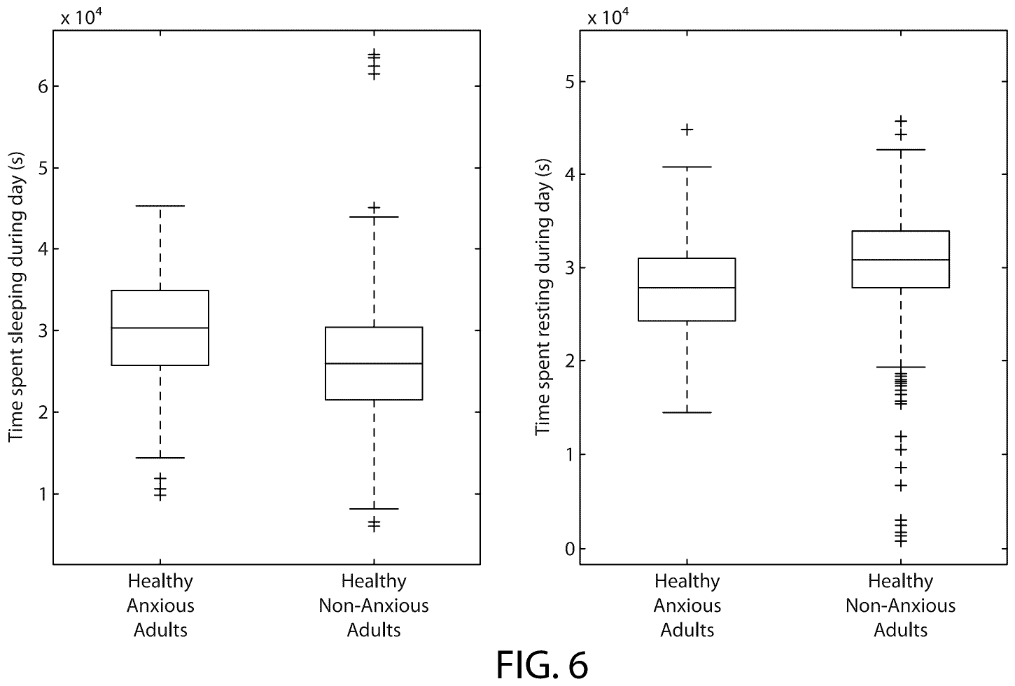

Anxiety is a generalized marker that may be related to DISHA metrics such as disorientation, changes in social interactions, sleep-wake cycle alterations, house soiling, and activity levels. Adult dogs were categorized as being prone to anxiety but otherwise healthy (n=6), or healthy and not prone to anxiety (n=26). As shown in FIG. 4, anxious dogs appear to sleep more and rest less. Further analysis of the timings of sleep behaviors show that this difference is expressed more prominently during the nighttime (FIG. 5), as daytime sleep and rest durations are more similar (FIG. 6).

In an aspect the disclosure provides a system for generating a multi-component aging index for an individual companion animal comprising digital biomarkers, traditional (biological) biomarkers and subjective assessment methods to predict phenotypic age and phenotypic age acceleration/deceleration (phenotypic age above or below chronological age), patterns in the rate of aging over time, and longevity in dogs and cats. The system for generating the multi-component index includes, without limitation, wearable devices to measure physical and specific forms of physical activity such as such as walking, running, resting, jumping, scratching, shaking, licking, chewing, sniffing or ground-tracking, exploration, tail wagging, play bowing, panting, circling, climbing (e.g., stair climbing), digging, vocalizing, abnormal gait, pacing, eating, drinking, urinating, defecating, vomiting, pain, lameness, reproductive and/or mating behavior, seizures, sleep health (including but not limited to sleep duration, sleep timing, sleep duration variability, sleep quality, sleep quality variability and the regularity of sleep timing), subjective assessment methods such as pet parent questionnaires and veterinarian questionnaires, and clinical characteristics, including, without limitation chronological age, body composition (such as the body condition score, body fat index or objectively measured body composition parameters), body weight, temperature, respiration, heart rate, pulse, and disease count.

In some embodiments, the index further utilizes environmental sensors to assess location, and thus location-based behaviors and activities, including, without limitation signs of health such as intake and appropriate or inappropriate output behaviors (such as occurrence of eating, drinking, urinating, defecating, vomiting), signs of illness such as gastrointestinal disease, urinary and/or kidney disease, dermatology disease, respiratory disease, neurological disease, cardiovascular disease, oral health, endocrine disease, infectious disease, immune function and/or inflammation and orthopedic disease and/or mobility/pain, and signs of emotional/cognitive health, including but not limited to fear, anxiety, stress, dementia, cognitive function, attention, and social interaction with humans and other animals.

In some embodiments, the index further utilizes two, three, four, five, six, seven, eight, nine or more of biomarker panels. The biomarker panels include, without limitation, at least one traditional biomarker chosen from clinical chemistry parameters, nutrition status markers, blood cell counts, genetic markers, chromosomal changes, gene expression markers, endocrine markers, brain health markers, stress markers, immune function and/or inflammation markers, metabolic markers, markers of organ function (for example, liver, heart, lungs, etc.), neurological parameters, body composition parameters, and/or microbiome parameters such as composition, diversity, function, etc.). The biomarker panel may include at least one traditional biomarkers selected from, but not limited to, albumin, creatinine, glucose, lymphocyte percent, mean cell volume, red cell distribution width, alkaline phosphatase, white blood cell count, SDMA, circulating peptides such as Aβ42, circulating postbiotics, immunoglobulins such as immunoglobulin M, growth hormone (GH)/insulin growth factor-1 (IGF-1), and DNA biomarkers including SNPs). The index could also include measures of the physical body such as body weight, indicators of body composition such as the body condition score or body fat index, as well as other health parameters, including physical activity and mobility, sleep parameters, behavioral parameters such as the frequency or duration of behaviors, pain measures, cognition measures, location and/or proximity information such as the frequency and duration of presence in a particular area, temperature, and subjective pet parent-reported information. See the below non-limiting examples for use of traditional biomarkers. The examples below include non-limiting examples of analysis that uses multiple traditional biomarkers to predict phenotypic age and aging acceleration/deceleration. They show how individual metrics differ between cats and dogs who do and do not survive a 3 year observation period. The examples use data on hundreds of dogs and cats from the PNC colony and observes them over a period of years. Also shown, in a different population, dogs at least 5 years of age, is how individual parameters vary in these dogs. The examples show which blood markers have a high proportion of unusually high or unusually low measurements in a population of older dogs. This analysis shows that elevated or decreased values of certain biomarkers may be common in older dogs and suggests an association between the parameter and age when considering parameters one at a time, and looking cross sectionally (at a moment in time, not over a period of years).

Traditional biomarkers include common biochemical parameters.

Common biochemical parameters measured in older dogs indicate deviations from normal values and may be useful markers for aging. For example, in a population of approximately 485 non fasted healthy dogs aged 5 and up enrolled from veterinarian clinics across the US the following distributions of normal and abnormal blood values were observed:
Albumin
   a. High: 2.9%
   b. Low: 0.6%
   c. Normal: 96.5%
Glucose (non-fasting)
   a. High: 0.0%
   b. Low: 4.3%
   c. Normal: 95.7%
Magnesium
   a. High: 11.1%
   b. Low: 0.4%
   c. Normal: 88.5%
Phosphorous
   a. High: 0.0%
   b. Low: 5.4%
   c. Normal: 94.6%
AST (aspartate aminotransferase)
   a. High: 0.0%
   b. Low: 5.4%
   c. Normal: 94.6%
GGT (Gamma Glutamyl Transferase)
   a. High: 0.8%
   b. Low: 19.8%
   c. Normal: 79.4%
BUN (Blood urea nitrogen)
   a. High: 2.5%
   b. Low: 1.0%
   c. Normal: 96.5%
TRIG (Triglycerides)
   a. High: 18.6%
   b. Low: 0.6%
   c. Normal: 80.8%
CHOL (Cholesterol)
   a. High: 15.3%
   b. Low: 0.0%
   c. Normal: 84.7%
GLOBU (Globulin)
   a. High: 3.7%
   b. Low: 0.0%
   c. Normal: 96.3%
CO2 (Bicarbonate)
   a. High: 0.0%
   b. Low: 3.1%
   c. Normal: 96.9%
ANGAP (Anion gap)
   a. High: 0.2%
   b. Low: 16.5%
   c. Normal: 83.3%
RBC (Red blood cells)
   a. High: 5.6%
   b. Low: 0.0%
   c. Normal: 94.4%
HCT (Hematocrit)
   a. High: 10.3%
   b. Low: 0.0%
   c. Normal: 89.7%
HGB (Hemoglobin)
   a. High: 8.4%
   b. Low: 0.0%
   c. Normal: 91.6%
MCV (Mean corpuscular volume)
   a. High: 10.1%
   b. Low: 4.7%
   c. Normal: 85.2%
PLT (Platelets)
   a. High: 4.3%
   b. Low: 0.4%
   c. Normal: 95.3%
ALKP (Alkaline phosphatase)
   a. High: 6.4%
   b. Low: 6.4%
   c. Normal: 87.2%

At least one of the above may be included as a traditional biomarker in embodiments herein. For example, by looking at these traditional biomarkers for a given dog, the dog's probable age category can be determined in embodiments herein. As an example, the normal number of healthy dogs with high magnesium is 0. If a healthy dog is found to have high magnesium, the data from healthy dogs shows those with high magnesium are disproportionately older dogs. Some older dogs still have normal magnesium, but if a healthy dog is found to have high magnesium, there is an increased probability that this dog should be considered to be in an “older” dog category, even dog's true age was unknown. A similar reasoning applies to HGB (hemoglobin). All the traditional biomarkers in the above list are shown because the distribution of these biomarkers in older healthy dogs differs from what we would expect in young healthy dogs. Traditional biomarkers also include those in Tables 11a and 11b, below. Some embodiments include one or more of the traditional biomarkers in Table 11a and/or 11b. Some embodiments include methods or systems utilizing one or more of the traditional biomarkers in Table 11a and/or 11b as in the below examples. In some embodiments, traditional biomarkers utilized for feline subjects include HCT, MCH, WBC, RDW, Bun/Creat, BUN, Chloride, Sodium, and animal age. In some embodiments, traditional biomarkers utilized for feline subjects include RBC, MCHC, MCH, MCV, RDW, Chloride, and Triglycerides. In some embodiments, traditional biomarkers utilized for feline subjects include Creatinine, Glucose, MCV, Albumin, RDW, Lymphocytes, WBC, ALP, and animal age. In some embodiments, traditional biomarkers utilized for canine subjects include MCHC, MCH, RDW, ALT, Albumin, Chloride, Sodium, Eosinophils, Lymphocytes, and animal age. In some embodiments, traditional biomarkers utilized for canine subjects include RBC, MCH, RDW, ALT, and Na/K ratio. In some embodiments, traditional biomarkers utilized for canine subjects include Creatinine, Glucose, MCV, Albumin, RDW, Lymphocytes, WBC, ALP, and animal age.

In some embodiments, a traditional biomarker is one that is distributed in older healthy animals differently from what is expected in young healthy animals.

In some embodiments, the index further utilizes epigenetic markers, including DNA methylome data in addition to or instead of traditional biomarkers.

In some embodiments, the index further incorporates data over a period of months or years, versus just a one-time sample. Such data includes, without limitation, the use of an expanded set of environmental sensors to observe proximity to pet parent, play, timing and frequency of feeding, timing and frequency of drinking, timing and frequency of urinating, timing and frequency of defecating, location of eating, drinking, urination and defecation, body posture (e.g., via pose estimation), tail position, body position, movement tracking over time, and incorporation of repeated measures of selected clinical, digital and biological data. In some embodiments, this provides for a phenotypic age index that is continuously updated as the pet is providing continuous data streamed through connected health devices as part of a connected health IoT ecosystem. Such devices include, without limitation, cameras, microphones, eye-tracking devices, touchscreens, smart litterboxes, smart pet beds, smart scales, smart feeders, smart collars or harnesses, smart clothing, smart thermometers, smart toys, smart water fountains, smart pet doors, in addition to one or a plurality of wearable sensors with accelerometer/gyroscope/magnetometer/thermometer/light sensors/Wi-Fi and Bluetooth capabilities and proximity beacons. Machine learning algorithms are implemented on the smart devices (including the wearable sensor or other smart devices), in ancillary technologies via distributed computing topology (“edge computing”), or in the cloud.
In some embodiments, a system and/or a method herein comprises a combination of at least two, three, four, five, six, seven, eight, or nine biomarkers to diagnose and/or recommend suitable treatment plan for the respective pets. The biomarkers may be tradition biomarkers. The biomarkers may be selected from those in the below examples. For example, in some embodiments, at least two, three, four, five, six, seven, eight or nine of the biomarkers are selected from the group consisting of Creatinine, HCT, MCH, MCHC, RBC, WBC, RDW, Bun/Creat ratio, BUN, Chloride, Sodium, Triglycerides Glucose, MCV, Albumin, RDW, Lymphocytes, Eosinophils, ALT, and Na/K ratio.
In some embodiments, traditional biomarkers utilized for canine subjects according to the present invention include any combinations of at least two, three, four, five, six, seven, eight, and nine of Creatinine, Glucose, MCV, Albumin, RDW, Lymphocytes, WBC, basophils, eosinophils, monocytes, neutrophils, ALT, ALP, chloride, cholesterol, globulin, albumin to globulin ratio, phosphorus, magnesium, sodium, potassium, total protein, and triglycerides.
In some embodiments, the methods according to the present invention may consider any combinations of 3, 4, 5, 6, 7, 8, 9, 10, 11, 12, 13, 14, 15, 16, 17, 18, 19, 20, 21, 22, 23, 24, 25, 26, 27, 28, or 29 to 30, 31, 32, 33, 34, 35, 36, 37, 38, 39 or 40 of any traditional biomarkers. In some embodiments, the methods according to the present invention may consider any combinations of three to 30, 31, 32, 33, 34, 35, 36, 37, 38, 39 or 40 of any traditional biomarkers. The traditional biomarkers may be selected from those herein. The traditional biomarkers may be selected from those listed above. In some embodiments, the index further utilizes epigenetic markers, including DNA methylome data in addition to or instead of the individual or combinations of traditional biomarker(s).
In some embodiments, the phenotypic index may include any combinations of two, three, four, five, six, seven, eight or nine data points over a period of months or years, versus just a one-time sample. Such data points may be obtained, without limitation, from the use of an expanded set of environmental sensors to observe proximity to pet parent, play, timing and frequency of feeding, timing and frequency of drinking, timing and frequency of urinating, timing and frequency of defecating, location of eating, drinking, urination and defecation, body posture, tail position, body position, movement tracking over time, and incorporation of repeated measures of selected clinical, digital and biological data.

### Wearables

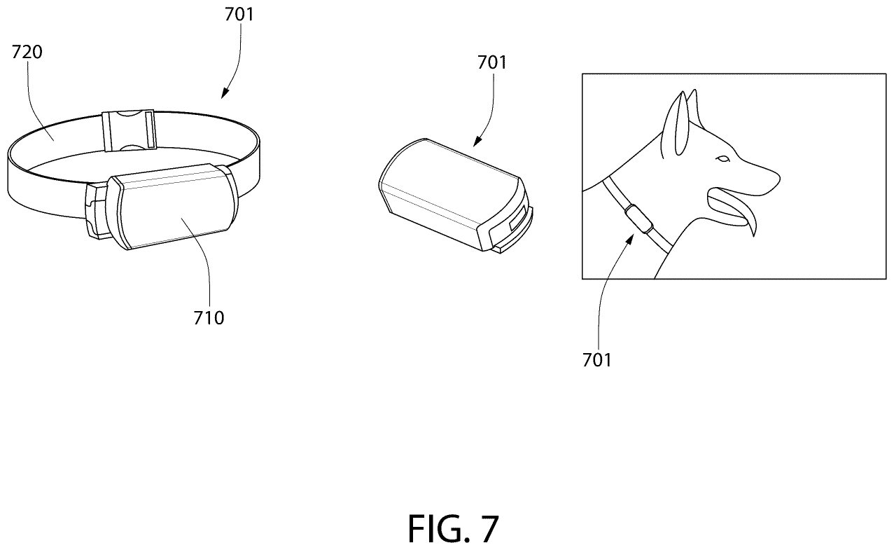

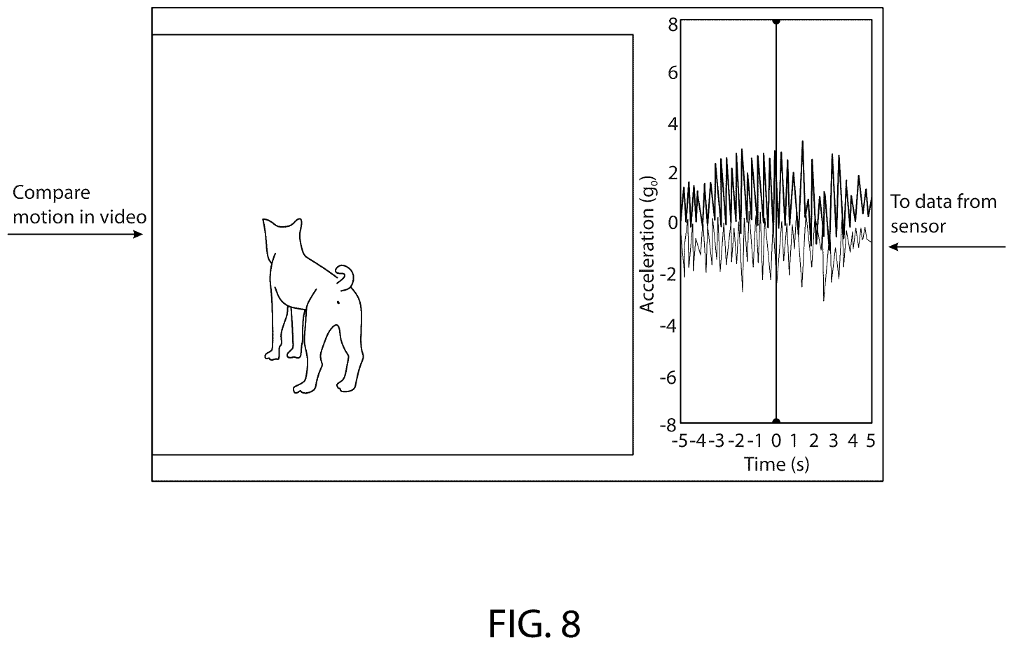

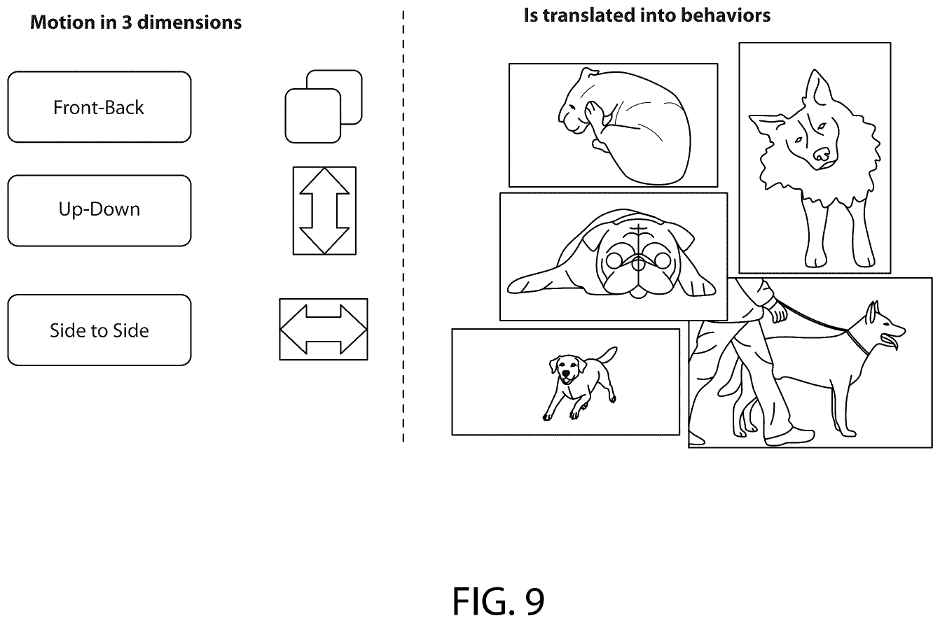

A wearable sensor is a lightweight digital tracker worn on the collar (See FIG. 7). Referring to FIG. 7, a wearable sensor 701 illustrated including a sensor 710 fixed to a collar 720. The wearable sensor 710 provides information on the dog's movements that is helpful to understanding the dog's health. The wearable sensor 701 is illustrated in use to capture movement in three dimensions in FIG. 8. Referring to FIG. 9, this 3D motion is translated into axes of motion, which may include front-back, up-down, and side to side. The axes of motion may be indicative of behaviors; for example, sleep, rest, walking, running, scratching, shaking, etc.

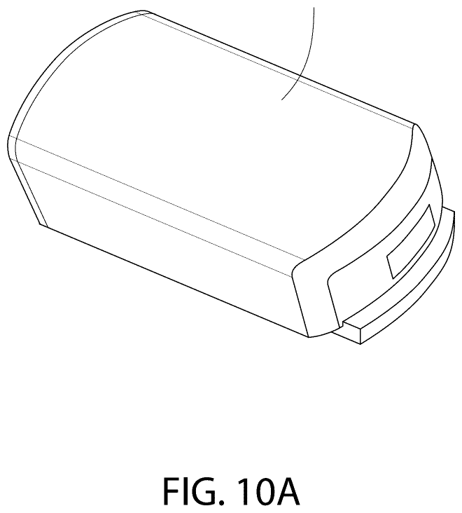

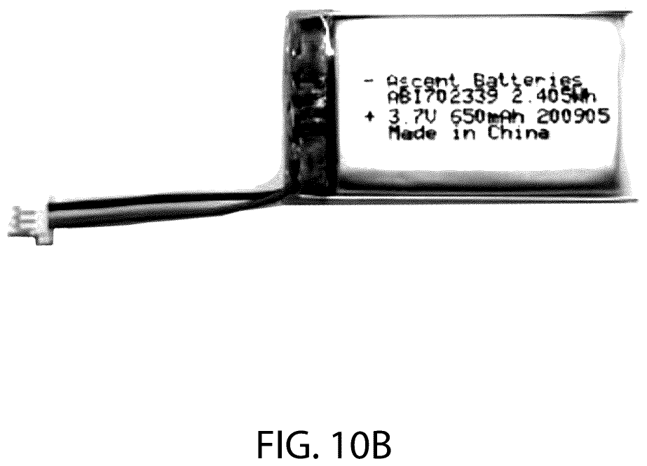

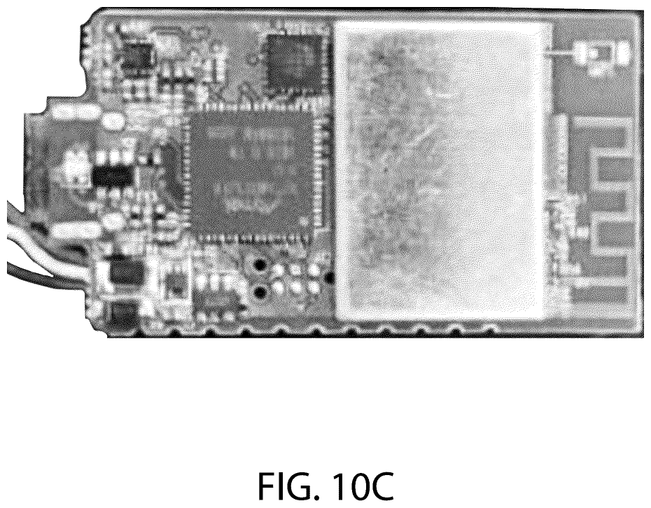

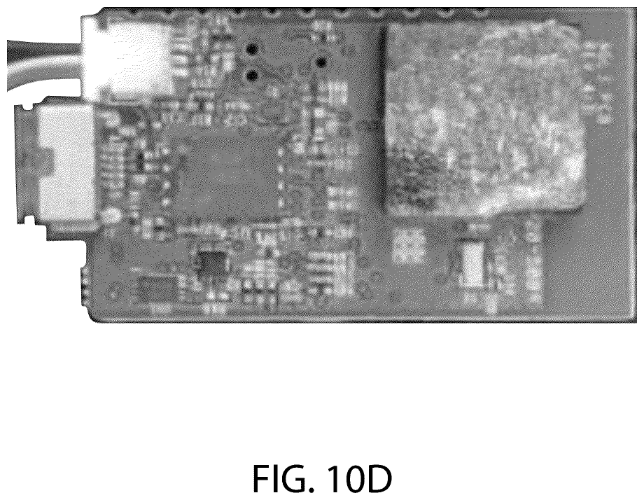

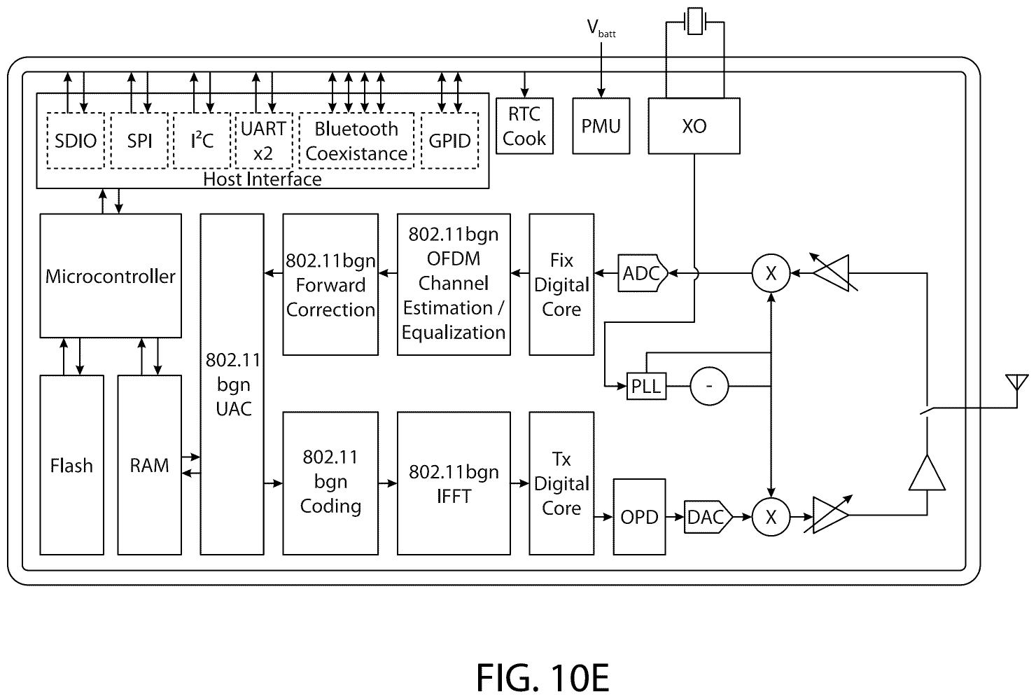

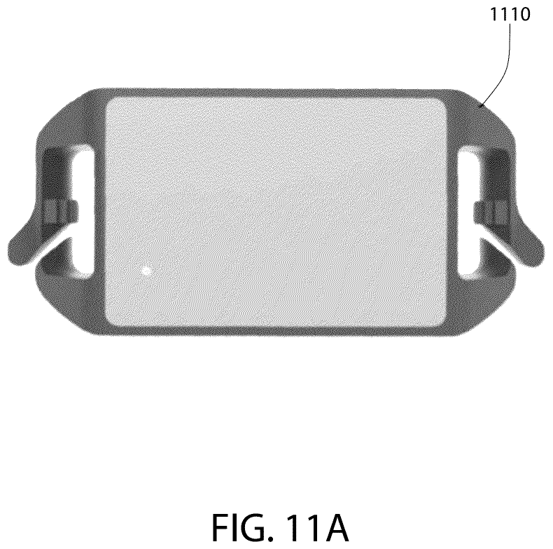

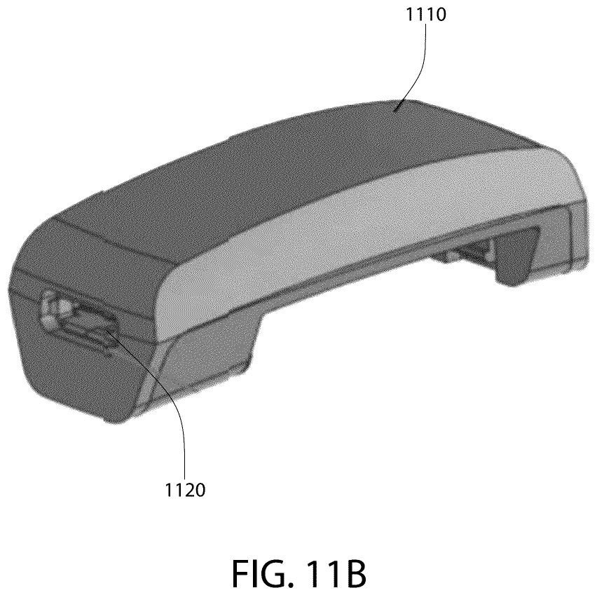

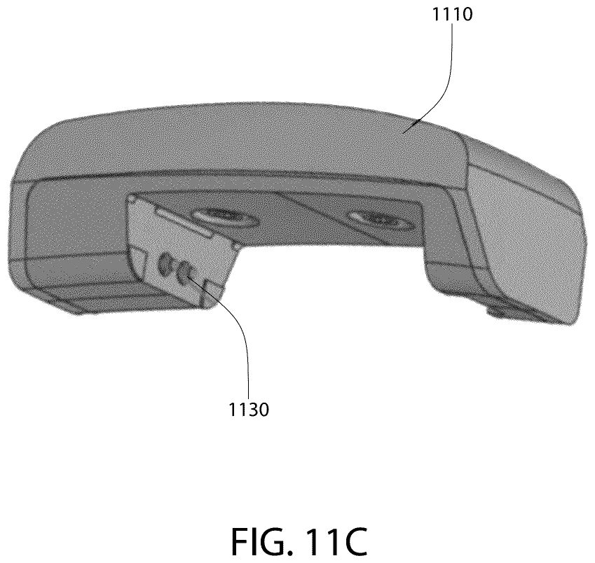

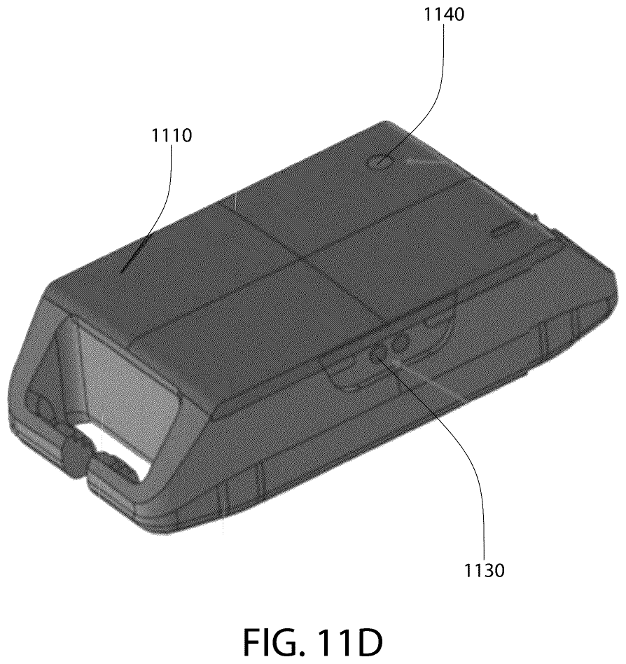

A Collar Mounted Activity Sensor (CMAS) is a wearable sensor. A CMAS in some embodiments includes an accelerometer. The accelerometer, in some embodiments, is capable of capturing motion data at 100 Hz. Referring to FIG. 10A, a sensor 1010 is in a configuration of a CMAS of some embodiments herein. Despite its small size, some embodiments of a CMAS may contain 130 electrical components, six axes of motion detection provided by an accelerometer (100 Hz sampling acceleration) and gyroscope (50 Hz sampling gyro), Wi-Fi and BTLE communications, 2 GB memory with a 256 KB flash, FIFO data output and about 10-day battery life. See FIGS. 10B-10E. Referring to FIGS. 11A-11D, another embodiment of a sensor 1110 is illustrated comprising a charger and incorporating HPN1 technology. The sensor 1110 as illustrated includes a USB port 1120, charging contacts 1130, an light pipe 1140. The sensor 1110 may utilize only 59 electrical components to provide 6 axes of motion detection with an accelerometer and gyroscope providing variable sampling in a single component, combined Wi-Fi and BTLE communication, 8 GB flash memory, FIFO data output, about 10-day battery life, proximity detection, smaller footprint and weight, and dual collar attachment options. A wearable sensor in some embodiments comprises the CMAS of FIG. 10A.

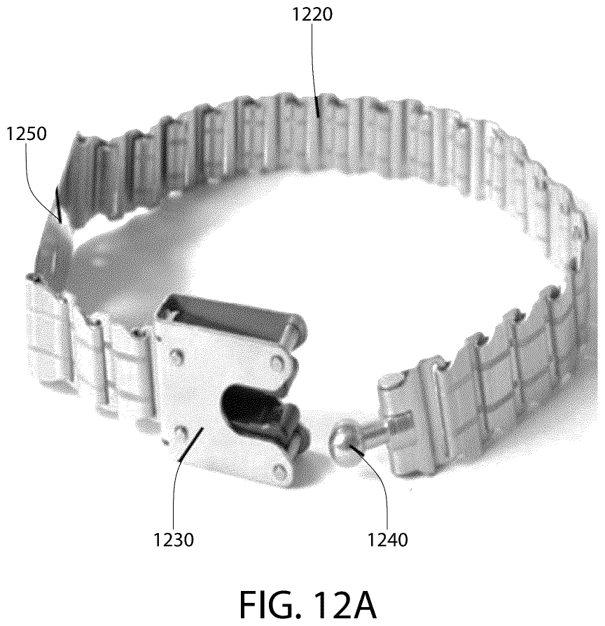

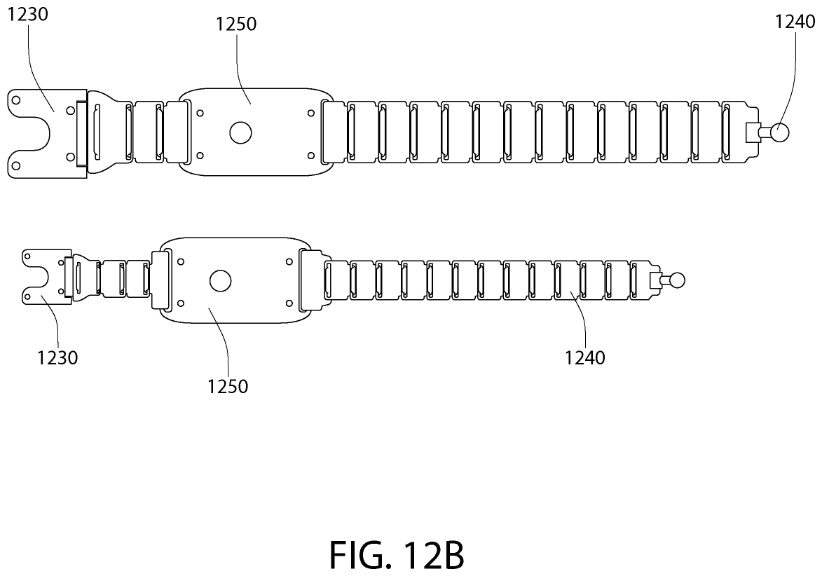

Referring to FIGS. 12A-12B. an embodiment comprising a durable collar 1220 is illustrated. The durable collar 1220 may be a break-away collar. The durable collar 1220 may be sized appropriately for each pet's physical proportions and weight as needed to provide a safe and practical attachment point for collar-mounted wearable sensors. The durable collar 1220 may comprise a ball 1240 and catch 1230 latch mechanism, and a sensor mount 1250. In an embodiment, the durable collar 1220 is a metal collar designs, which solves problems of safety, practicality and durability and allowed a custom fit for each pet.

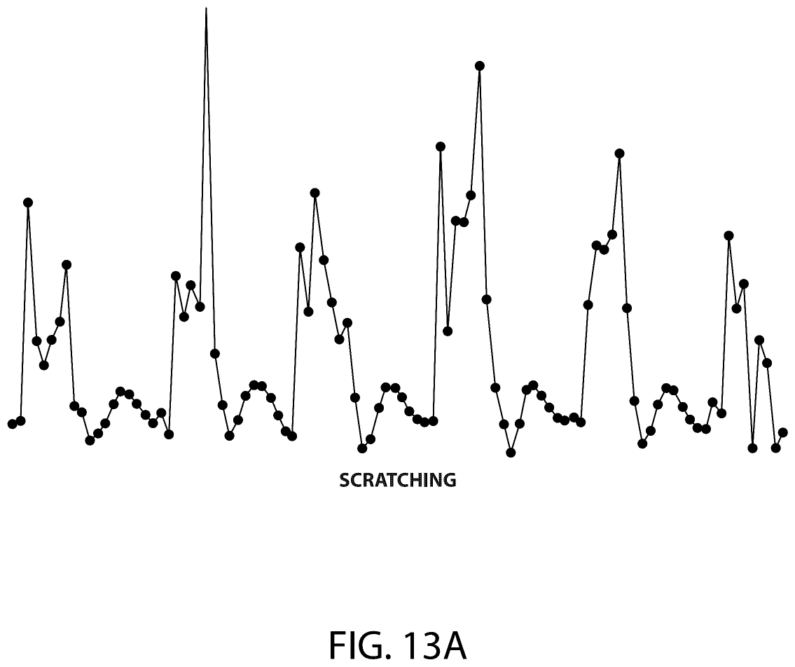

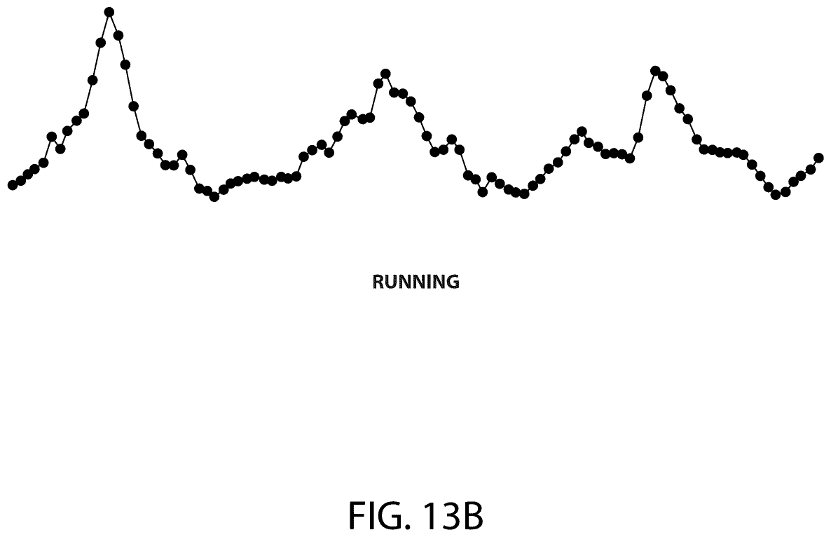

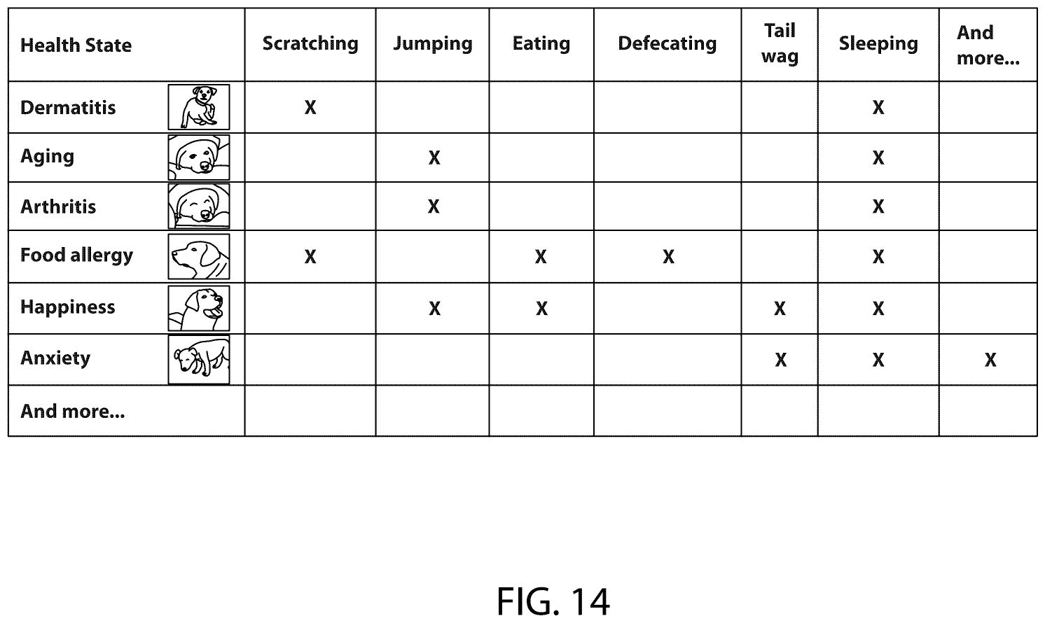

The 3D motion data collected utilizing such sensors provides training material for algorithms as well as the ongoing measurements needed to quantify behaviors. The high-resolution data is transmitted in great detail (See FIGS. 13A and 13B). Frequent readings provide easier classification into behaviors and models or conclusions from frequent readings are likely to be more highly sensitive, specific and accurate. Models that use multiple variables to predict changes in behavior patterns may be used in embodiments to characterize health states. See, for example, FIG. 14, which illustrates a grid cross referencing a pets health state, emotional state, and/or cognitive state (e.g., dermatitis, aging, arthritis, food allergy, happiness, anxiety, and/or others versus symptoms for each condition (e.g., scratching, jumping, eating, defecating, tail wag, sleeping, and/or others. Strong algorithm validation and performance metrics generate confidence in health claims (see e.g., Griffies et al., BMC Veterinary Research (2018) 14:124), which is incorporated herein by reference as if fully set forth.

The disclosure relates to a sensor embedded in, attached to, or mounted on a collar and a system comprising the same, wherein the device comprises a sensor configured to measure a heart rate, cardiograms, pulse, such sensors positioned within a housing. In some embodiments the disclosure relates to a device comprising a sensor configured to measure a heart rate, cardiograms, pulse, respiration rate, and such sensors are positioned within a housing, with the housing physically attached to a collar. In some embodiments, the collar is wearable or designed to be worn by a pet. In some embodiments, the device is operably connected to a controller, a display, and computer program product with executable code to perform any of the method steps disclosed herein.

### Computer Vision

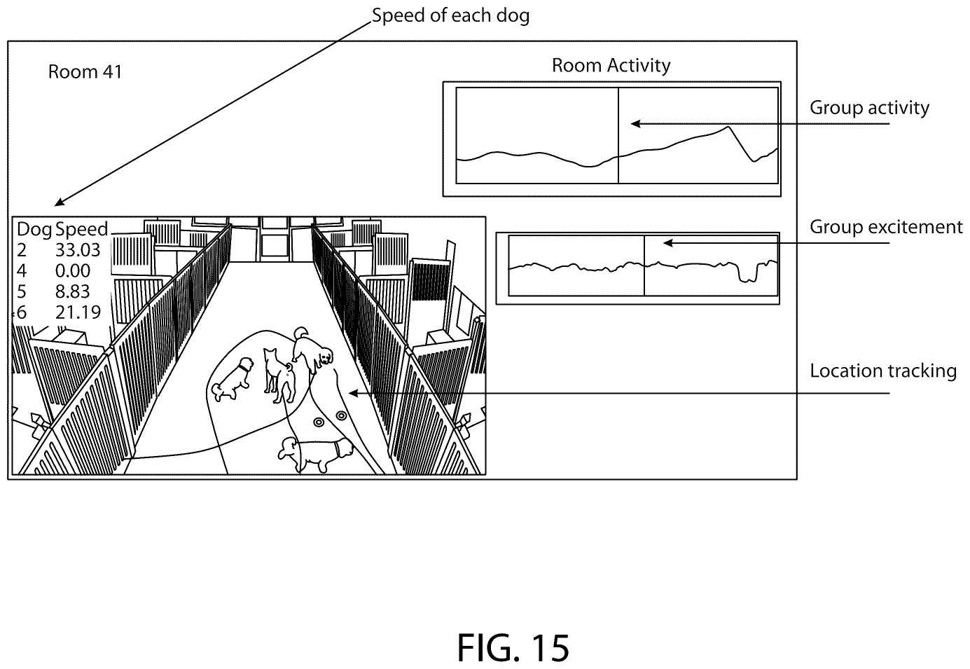

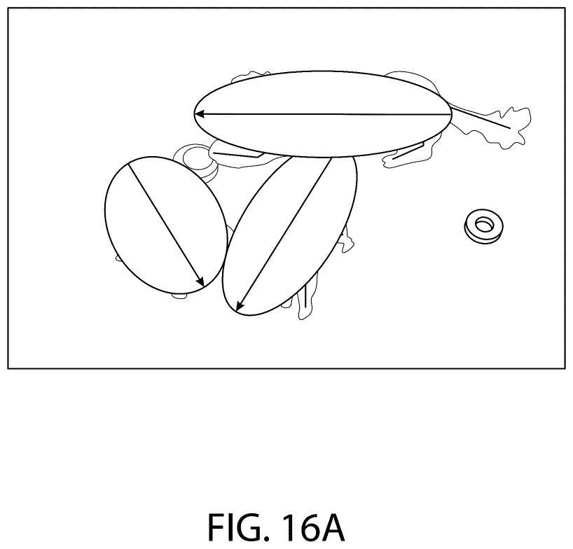

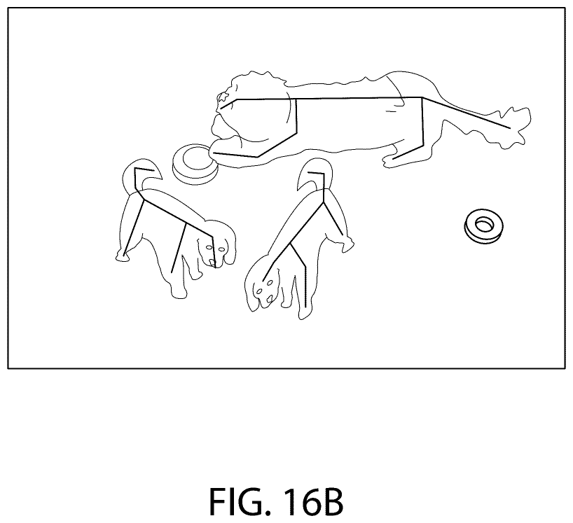

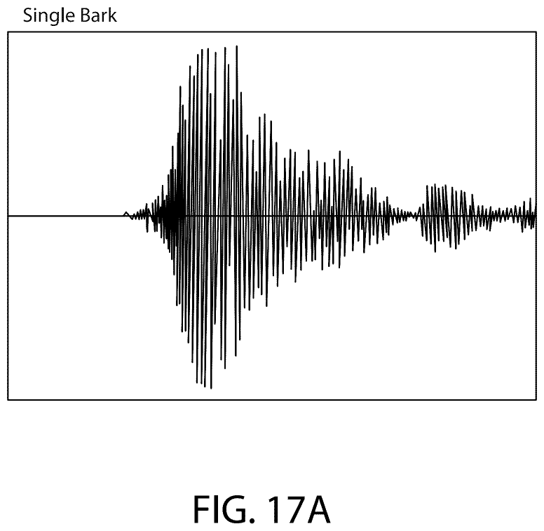

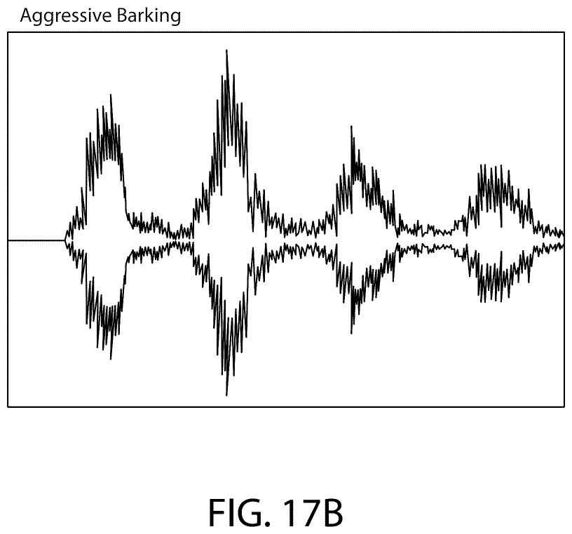

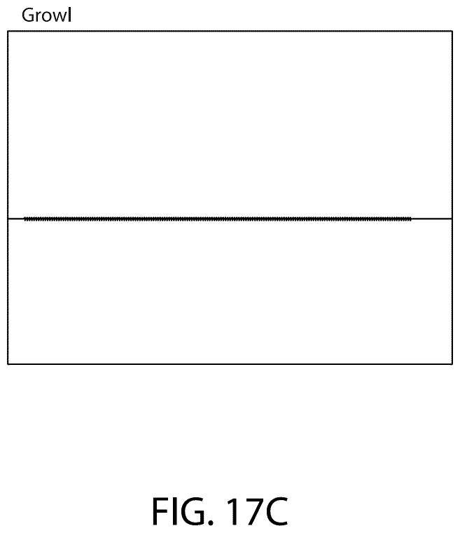

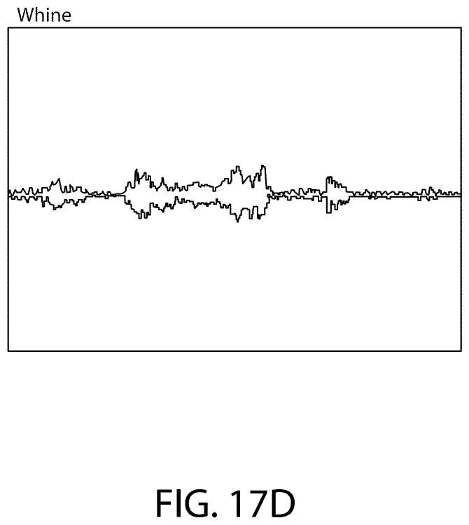

Embodiments include computer vision, which is a field of artificial intelligence that enables computers to derive useful information from digital images, videos, or other visual inputs, facilitating decision making based on the content of the visual. One such use is to identify pet behavior from collections of images. Computer vision on groups of pets is complex, but important to understand pet behaviors. An example is shown in FIG. 15, Detecting interactions is the first step to characterization. Neural networks can segment regions of an image that contain individuals and further assign and track each individual uniquely. When individual come into close proximity with each other, they can track and characterize interactions between them. As shown in FIGS. 16A and 16B, embodiments comprise reducing the complex geometry of a detected individual to an ellipse or similar bounding box can allow for characterization of the orientation of the dogs with respect to each other. In some embodiments, pose estimation is included and allows for further refinement of interaction characterization. In some embodiments, tail, head and ear position all provide signals between interaction partners. In some embodiments, the system can also monitor vocalizations to provide context to interactions (see FIGS. 17A-D). In some embodiments, pose estimation can also be used to characterize body language, which could contribute to emotional characterization, and be an input to phenotypic age model(s).

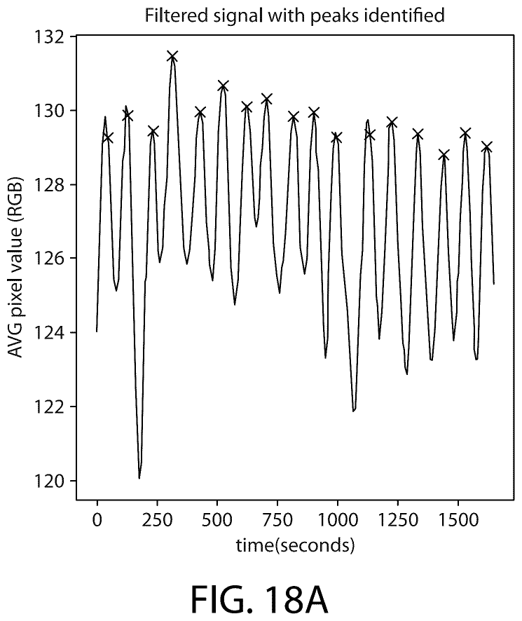

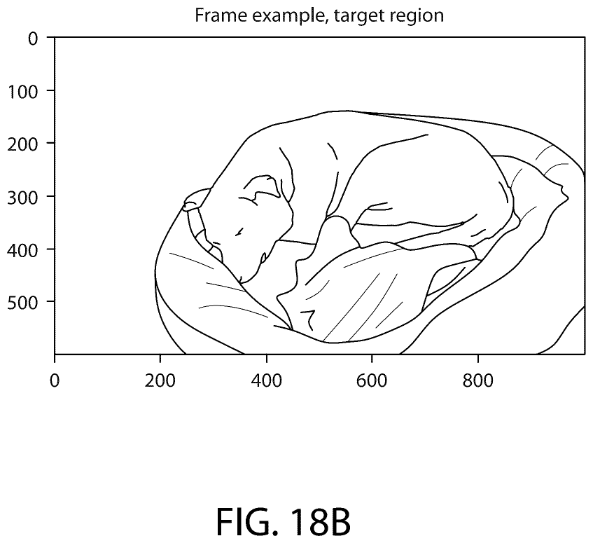

In some embodiments, computer vision is used to monitor parameters such as respiration rate to contribute to an evaluation of physical and emotional well-being. As shown in FIGS. 18A and 18B, an embodiment comprises computer vision that can focus on a target region on the pet and identify changes (e.g., in the case of dermatological issues). In some embodiments, computer vision may also be used to measure physical characteristics such as length, width, height, circumference, depth, volume, surface area, weight, mass, and the like. In some embodiments, computer vision may also be used to estimate, score or predict body composition parameters. Non-limiting examples of the body composition parameters include a Body Condition Score on 5 point or 9 point scales, Body Fat Index (e.g., a 20-70 scale), Muscle Condition Scoring, and Body Composition parameters. Non-limiting examples of Body Composition parameters include Total Mass (kg), Fat Mass (kg), Lean Mass (kg), Fat Mass (kg)/Lean Mass (kg), % Fat Mass, % Lean Mass, body fat distribution, lean mass distribution, water mass, water mass percentage, bone mineral density, bone mineral contents, volume of body compartments, frame size, and skeletal parameters.

### Smart Beds, Smart Rooms and Smart Toys

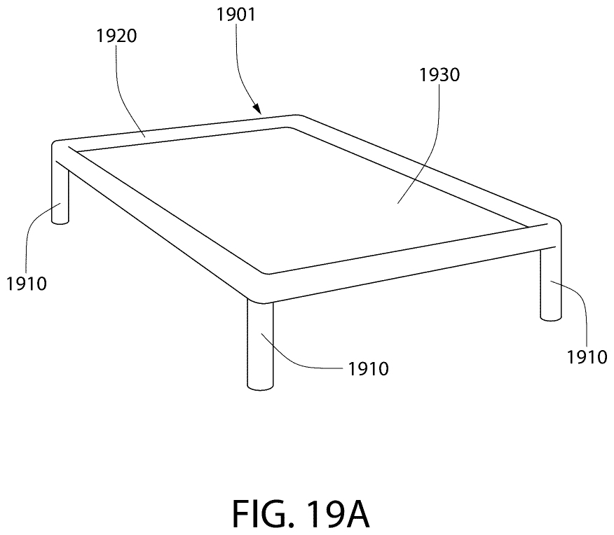

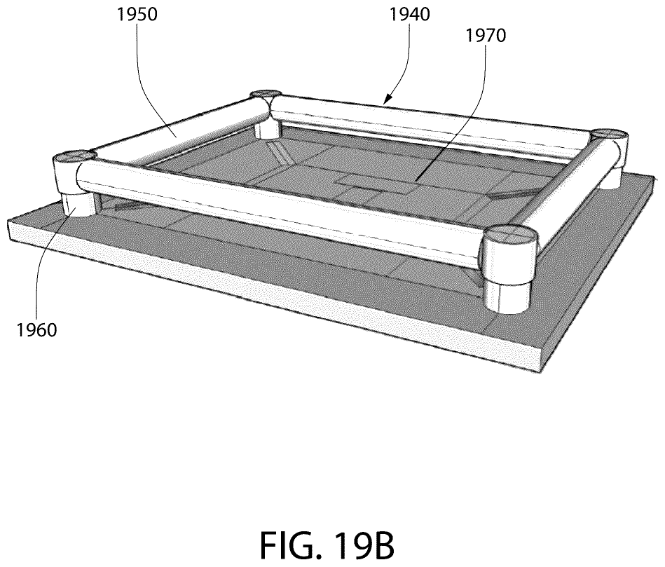

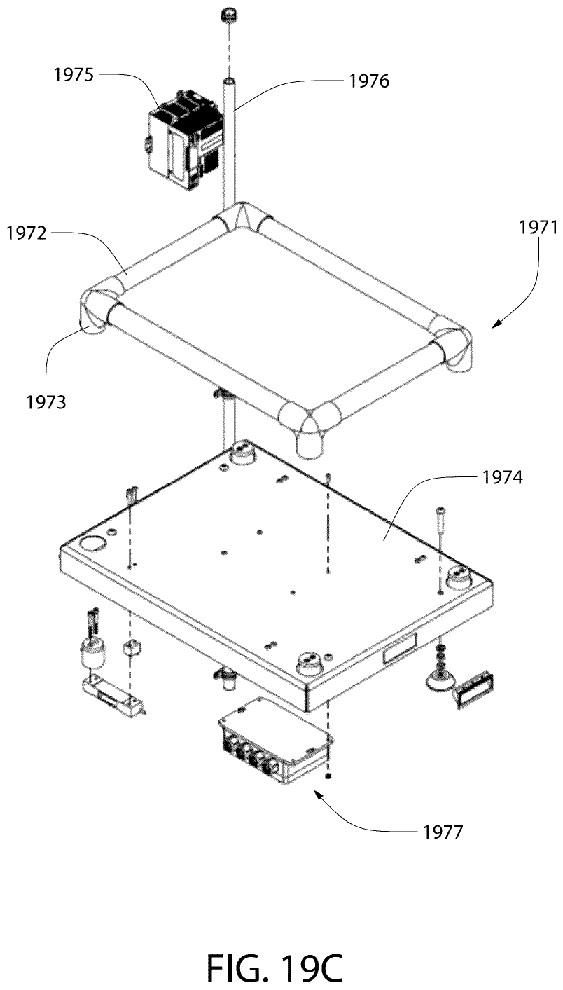

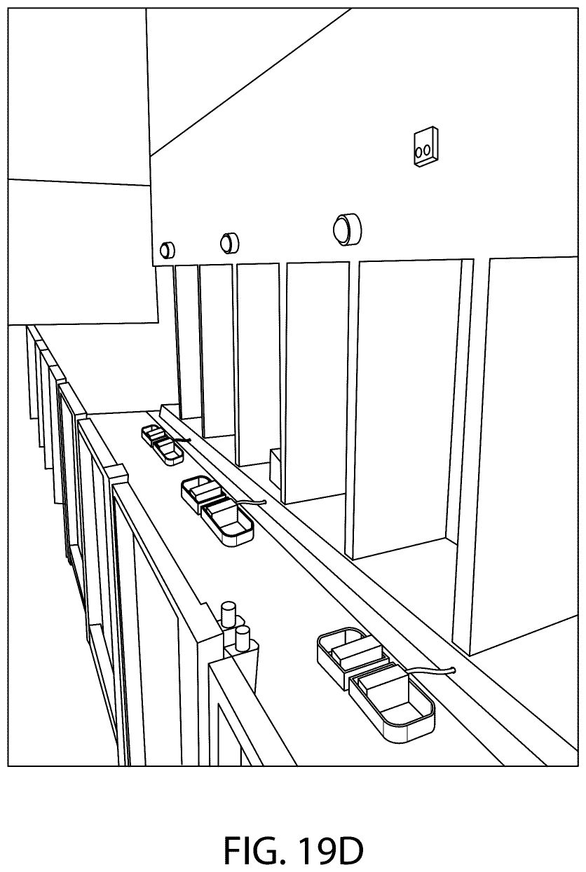

Some embodiments comprises a smart pet bed, which can be used to provide non-invasive monitoring of the pet's weight, temperature, heart rate and/or pulse, respiration rate, sleep parameters, audio parameters or vocalizations, and behavioral parameters such as social sleeping. Referring to FIGS. 19A and 19B, an embodiment of a smart bed is illustrated. FIG. 19A illustrates a smart bed 1901 comprising legs 1910, a frame 1920, and a mattress 1930 extending within the frame 1920. FIG. 19B illustrates an embodiment of a smart bed 1940 comprising a frame 1950, legs 1960 supported on a base 1970. A detailed view of an exemplary smart bed 1971 is shown in FIG. 19C. The smart bed 1971 may comprise a frame 1972, legs 1973, a base 1974, an electronics assembly 1975, and a mount 1976 for the electronics assembly 1975. The smart bed 1971 may further comprise sensory equipment 1977. The sensor equipment 1977 may comprise elements listed in the below Table B.

TABLE B
Items Illustrated in FIGS. 19A and 19B

| ITEM NO. | QTY. | PART NO. | DRAWING NO. | SUPPLIER | DESCRIPTION |
|---|---|---|---|---|---|
| 1 | 1 | A12620-01-001 | A12620-01-001 | BURGER & | CANINE BED BASE |
| | | | | BROWN | |
| 2 | 4 | A12620-01-002 | A12620-01-002 | BURGER & | LAOD CELL SPACER |
| | | | | BROWN | |
| 3 | 4 | A12620-01-003 | A12620-01-003 | BURGER & | KURANDA BED MOUNT |
| | | | | BROWN | |
| 4 | 1 | A12620-02-000 | A12620-02-000 | BURGER & | ELECTRONICS ASSEMBLY |
| | | | | BROWN | |
| 5 | 1 | A12620-03-000 | A12620-03-000 | BURGER & | SUMMING CARD |
| | | | | BROWN | ASSEMBLY |
| 6 | 4 | H1SPA22-10 | | HARDY | HARDY 10 kg SINGLE POINT |
| | | | | | LOAD CELL |
| 7 | 4 | 60655C73 | | MCMASTER- | ⅜-16 VIBRATION DAMPING |
| | | | | CARR | FOOT |
| 8 | 4 | 92146A031 | | MCMASTER- | ⅜ SS SPLIT LOCK WASHER |
| | | | | CARR | |
| 9 | 8 | 91847A031 | | MCMASTER- | ⅜-16 SS THIN HEX NUT |
| | | | | CARR | |
| 10 | 8 | 91292A144 | | MCMASTER- | M6x1 × 500 mm SSHCS |
| | | | | CARR | |
| 11 | 8 | 92095A244 | | MCMASTER- | M6x1 × 300 mm BSHCS |
| | | | | CARR | |
| 12 | 4 | 9294PA632 | | MCMASTER- | ⅜-16 × 2″ 55 BHGS |
| | | | | CARR | |
| 13 | 4 | 9294PA267 | | MCMASTER- | #10-32 × ⅝″ SS BHCS |
| | | | | CARR | |
| 14 | 4 | 9d278A411 | | MCMASTER- | #10-32 SS EXTERNAL |
| | | | | CARR | TOOTH LOCK WASHER |
| 15 | 2 | 1098A94 | | MCMASTER- | SNAP-IN RECESSED PULL |
| | | | | CARR | HANDLE |
| 16 | 1 | 79112K11 | | MCMASTER- | RIGID PVC CONDUIT, CUT |
| | | | | CARR | TO LENGTH |
| 17 | 3 | 3177TS5 | | MCMASTER- | VIBRATION DAMPING |
| | | | | CARR | CONDUIT CLAMP |

As illustrated in FIG. 19D, some embodiments comprise a smart room. A smart room may comprise cameras and an automated pet feeder system to monitor, alone or in conjunction with wearable or other smart devices, one or more of a pet's food intake and activity, which data may be used to determine the pet's caloric requirements. Some embodiments comprise smart toys. The smart toys, in some embodiments, are used as data collection instruments for detection of health-related endpoints. Exemplary, non-limiting, health-related endpoints comprise cognitive function, signs of aging, physical strength, and oral health. In some embodiments, a smart toy is used for saliva collection. In some embodiments, a smart toy can also be used to measure bite strength/force, mouth temperature, audio signals, video signals, pH and more, per the separate smart toy IR. In some embodiments, a smart room comprises microphones. In some embodiments, a smart room comprises one or more smart bed.

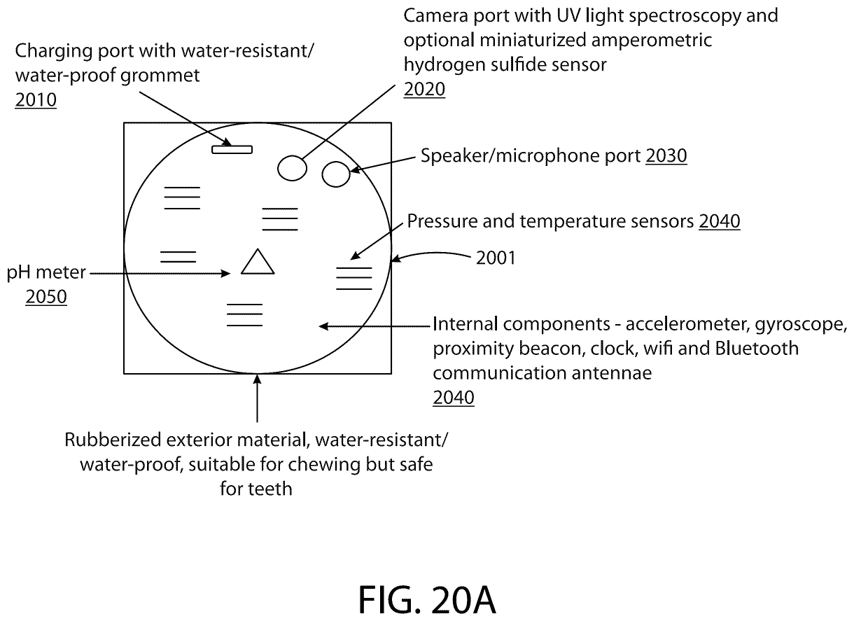

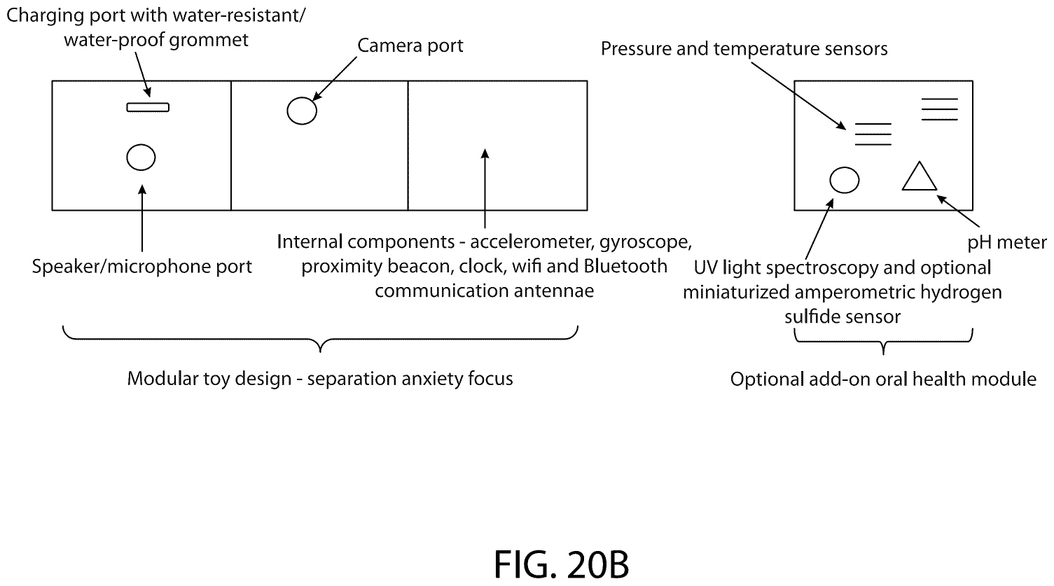

FIG. 20A illustrates an exemplary smart toy 2001. The smart toy 2001 comprises a charging port 2010, a camera port 2020, a speaker/microphone port 2030, pressure and temperature sensor 2040, internal components 2040 (which may comprises at least one of an accelerometer, a gyroscope, a proximity beacon, a clock, wifi, Bluetooth, or communication antenna), and a pH meter 2050. A smart toy 2001 may comprise a rubberized exterior material. The material may be water-resistant or water-proof. The material may be suitable for chewing but safe for teeth. FIG. 20B illustrates modular smart toy components. Any component of a smart toy may be housed on a similar or the same material as the exterior material of the smart toy. The modular components may be releasably engaged with a surface of the smart toy such that modular components may be switched. The left panel of FIG. 20B illustrates a modular component with a separation anxiety focus that comprises a charging port with a water resistant or water proof grommet, a camera port, a speaker/microphone port, and internal components (e.g., at least one of an accelerometer, a gyroscope, a proximity beacon, a clock, wifi, Bluetooth, and communication antennae. The right panel of FIG. 20B illustrates a modular component with an oral health module. The oral health module comprises pressure and temperature sensors, a pH meter, and a UV light spectroscopy sensor, and optionally a miniaturized amperometric hydrogen sulfide sensor. In some embodiments, a smart toy can also be used to measure bite strength/force, mouth temperature, audio signals, video signals, pH and more, per the separate smart toy IR.

### Algorithms

The following references provide examples of algorithms useful in the disclosure and are hereby incorporated by reference in their entirety. These algorithms may be in embodiments herein. WO2022/066282 describes an algorithm that can successfully and reliably recognize forward motion (gait) behaviors of a pet wearing a collar-worn device. WO2022/031513 uses a wearable device to determine (estimate) biometric data of a pet. In particular, this tool is an algorithm that uses measurements of the animal's activity to estimate energy expenditure (and thus caloric requirements and recommended feeding quantity) that is based on an objective assessment of activity. WO2022/072049 demonstrates near real-time assessment/continuous observation of response to nutrition therapy, estimation of energy intake and assessment of GI health and disease, assessment of stress, generalized illness and/or aging, and inappropriate elimination in cats and dogs. These algorithms can also be paired with other tools that measure location or proximity to provide assessments of the subject's location (indoors/outdoors).

### Customized Health, Diet and Nutrition

The measures and index according to the present disclosure provide guidance for the delivery of personalized health, diet and nutrition. For example, U.S. Provisional Application No. 63/241,173, and US Pre-grant Publication No. 20230073738, which are hereby incorporated by reference, disclose a method, device, and system including a sensor configured to capture image data relating to a pet for determining a body attribute of the pet, for example an ideal body weight (IBW), a body condition score (BCS) of the animal, a body fat index (BFI) of the animal, a weight or other attributes of the animal or part of the pet, including height, width, length, depth, diameter, radius, circumference, and the like.

In some embodiments, activity data recorded using wearable sensors may be used to estimate the energy expended during activity and the analysis of physical parameters and body composition may be used to refine these calculations. Body composition parameters and/or physical measurements and/or tools such as computer vision or other machine learning models and algorithms may be used to estimate weight targets and identify appropriate feeding algorithms based on weight gain, loss, or maintenance goals. Together with activity data, a customized feeding recommendation may be achieved.

Some embodiments comprise observation of a pet's interaction with food. Such an evaluation may be useful as a measure of ingestive behavior. Such an evaluation may be useful for such personalized, customized, and/or tailored health, diet and nutrition recommendations or follow on administrations of personalized, customized, and/or tailored health, diet and nutrition treatments. This includes the rate of feeding, time engaged with the food bowl, number of bites of food, size of the bites, speed of the bites, time spent sniffing or exploring, time spent near vs. away from the bowl, orientation of the pet toward the bowl vs. toward other people or objects in the environment, body posture, temporal parameters such as duration of time between presentation of food and the time that eating begins or number of bites per minute, time spent chewing, time spent eating, time required to finish the food, time spent in different postures, tail orientation and/or tail motion, breaks in feeding, vomiting, regurgitation or expulsion of food, presence of chewing, chewing in various parts of the mouth (ex., chewing exclusively on one side of the mouth as if to favor a part of the mouth due to pain or sensitivity), ratio of eating to other behaviors such as drinking, sniffing, etc., and facial expressions before, during and after feeding.

### Cats

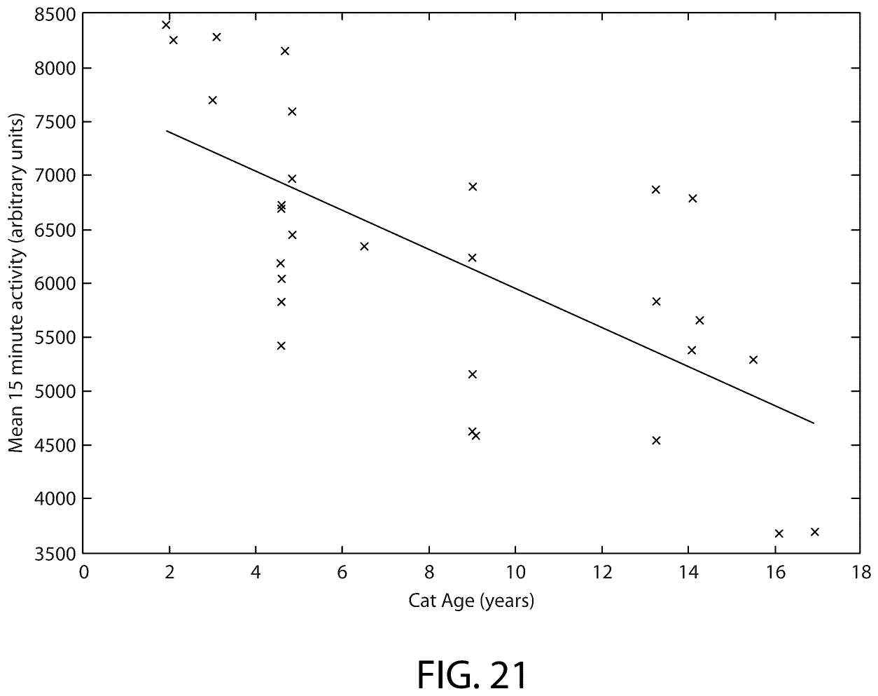

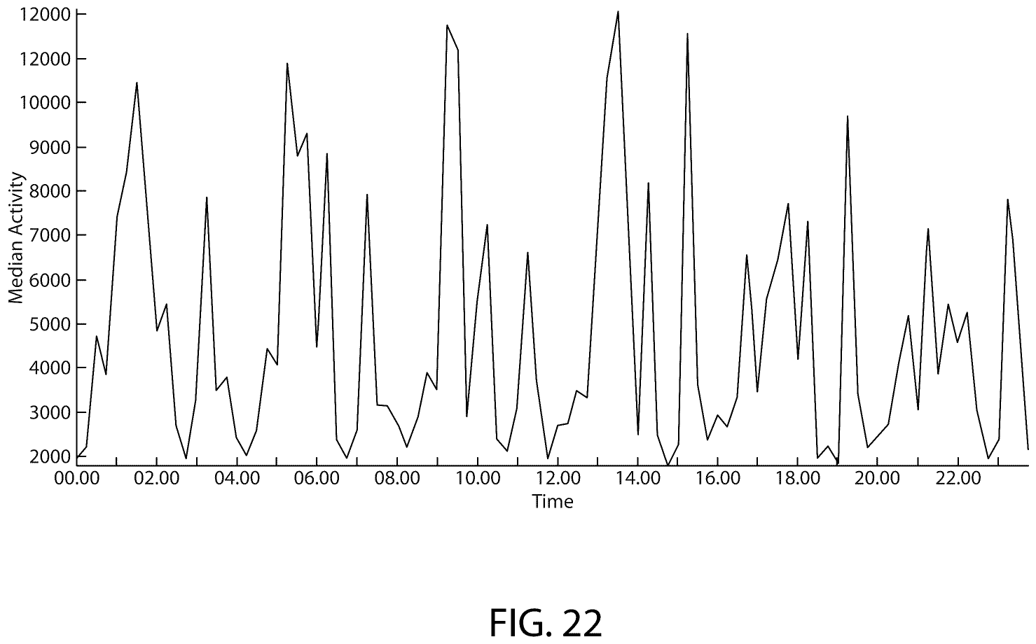

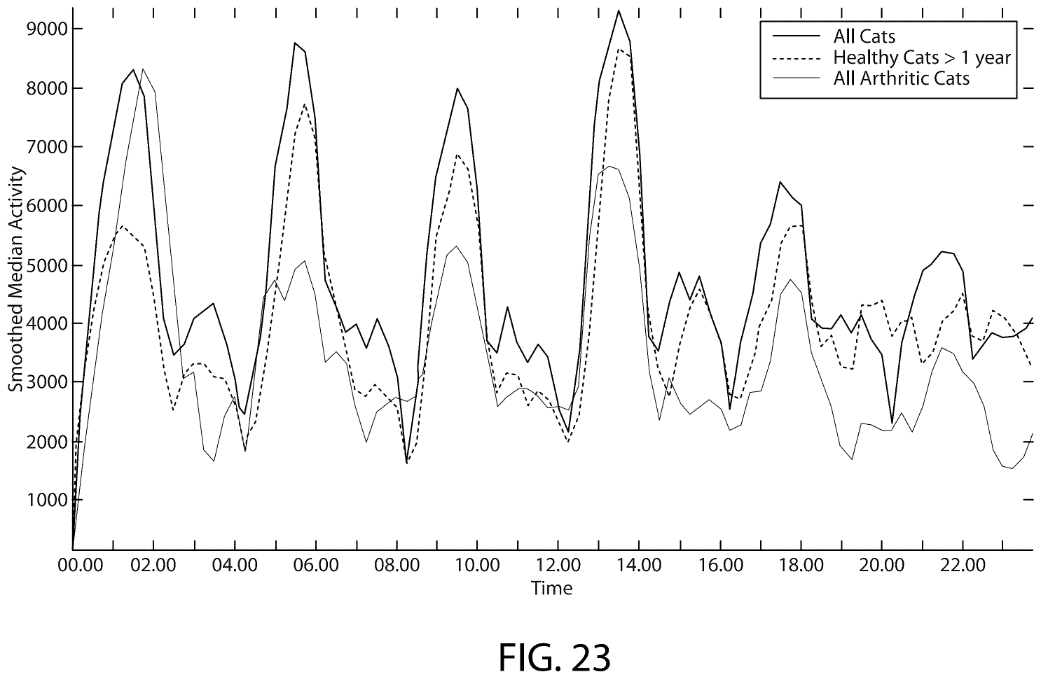

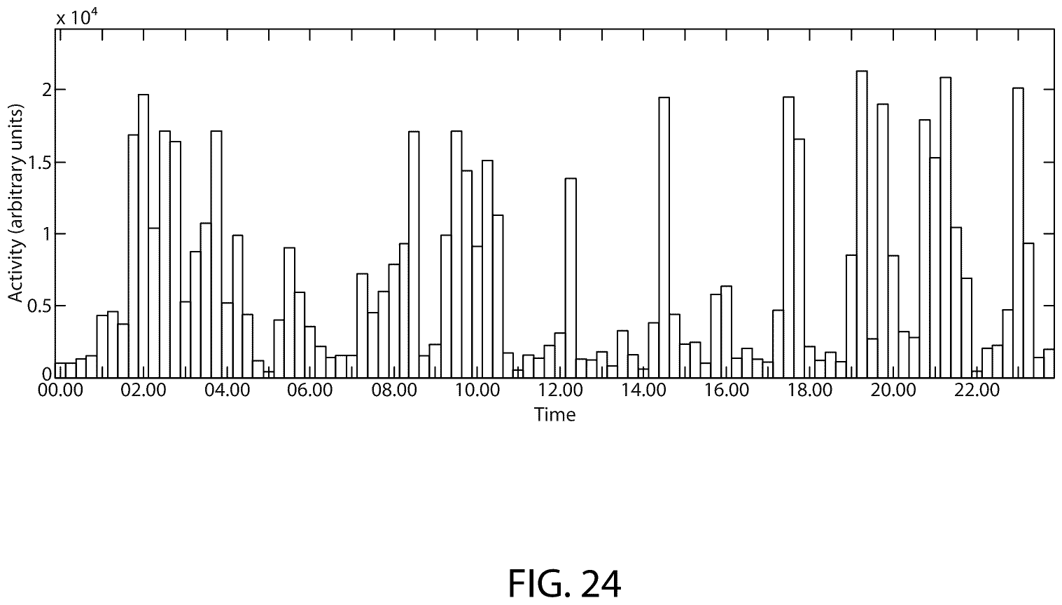

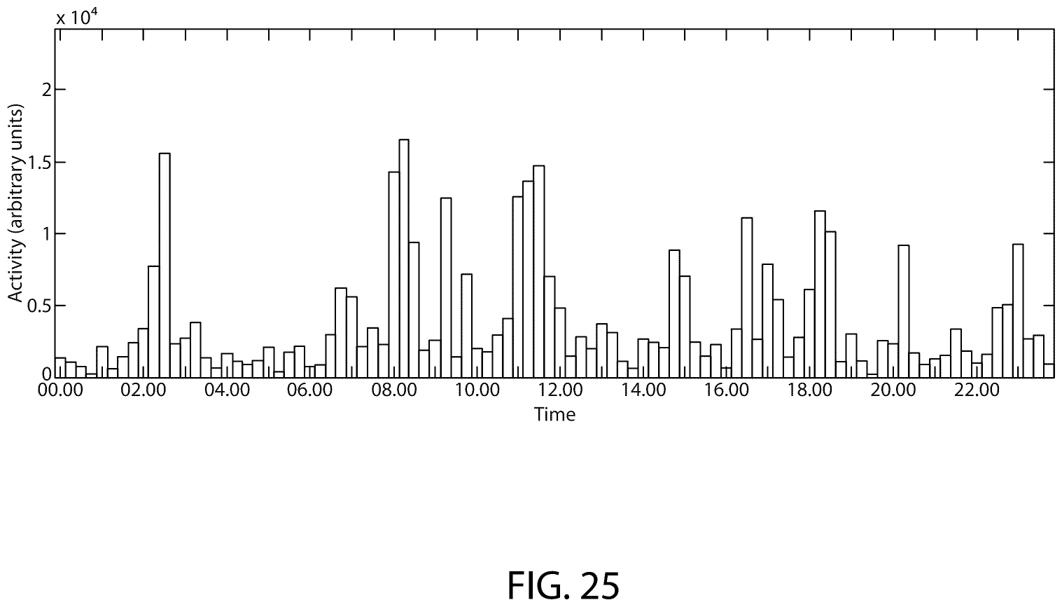

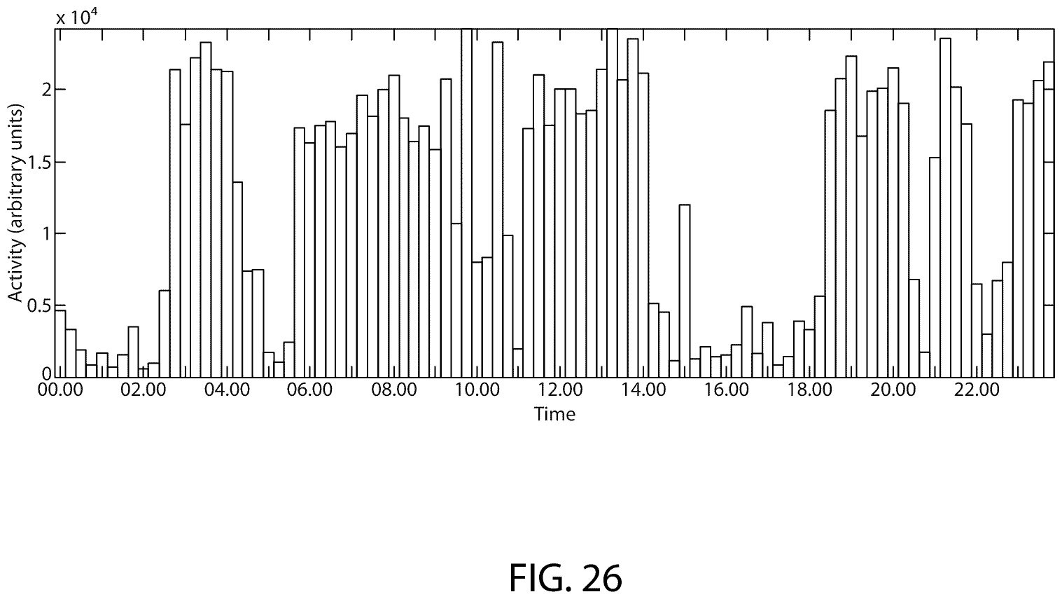

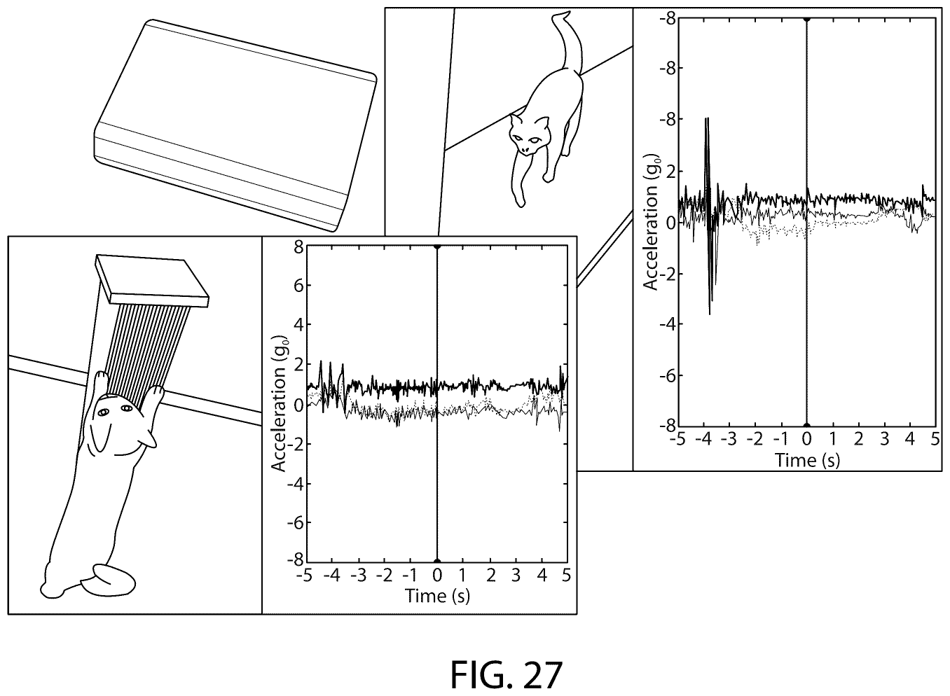

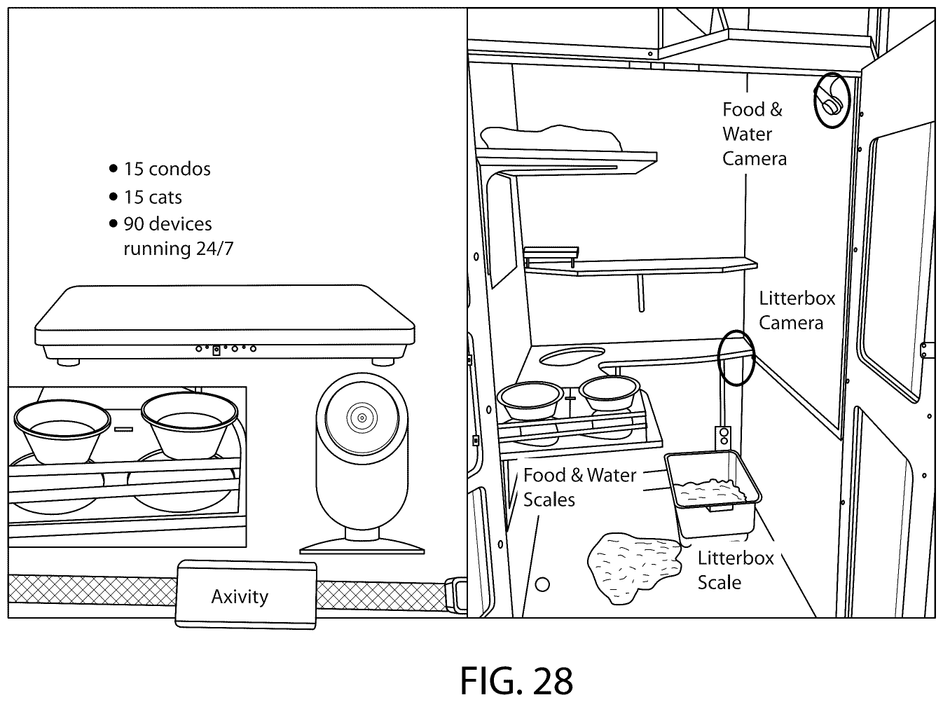

Although the system according to the disclosure has largely been described according to its implementation in dogs, those skilled in the art will immediately recognize that it is readily adaptable to cats. For example, FIG. 21 shows changes in activity in cats based on age. FIG. 22 shows a daily activity profile for an “average” cat. FIG. 23 compares daily activity profiles for all cats, healthy cats and arthritic cats. FIG. 24 shows daily activity of a healthy adult cat. FIG. 25 shows daily activity of a geriatric cat with past health issues. FIG. 26 shows daily activity of a five-month old kitten. FIG. 27 shows cat accelerometers in use. FIG. 28 shows a smart system, smart room, set up for cats.

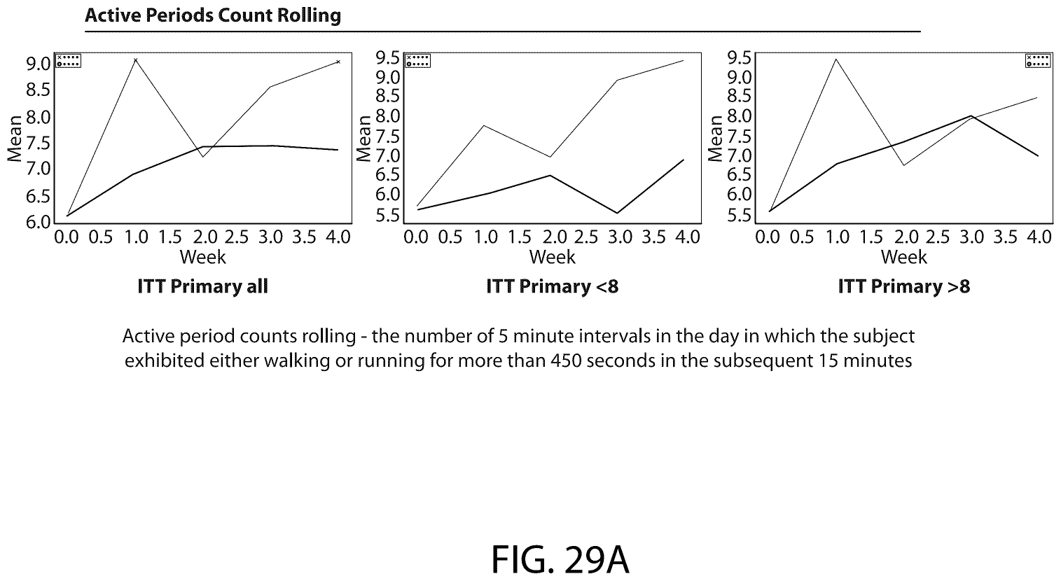

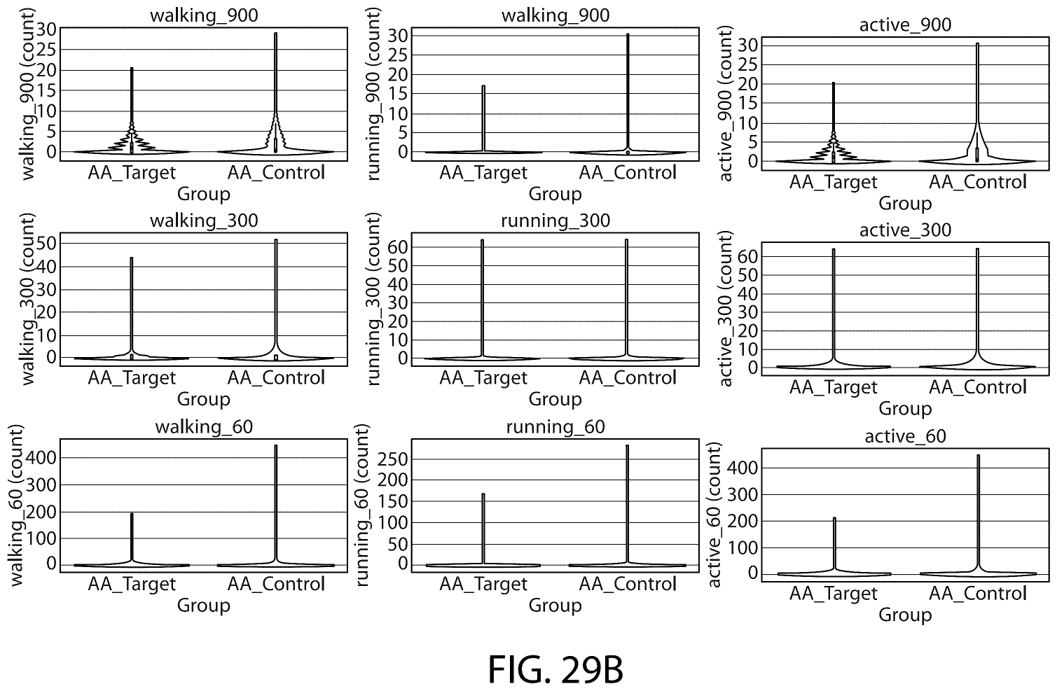

FIGS. 29A and B depict plots of session metrics for Aging and Non-aging. FIG. 29A shows line plots charting change from baseline in the number of 15 minute periods in which the subject was walking, running, or active per day for treatment and control groups. 29B shows violin plots visualizing the distribution of the count of dogs that exhibited behavior sessions for varying durations and behaviors.

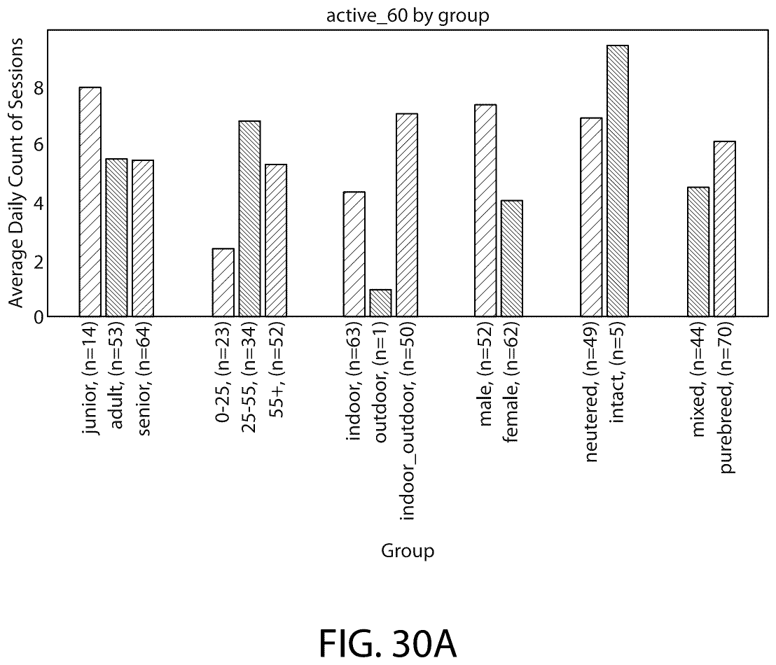

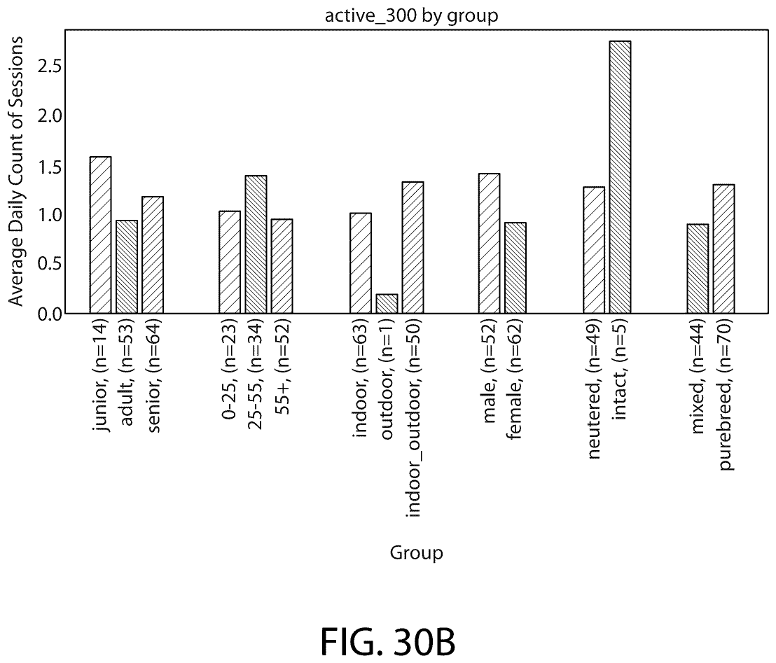

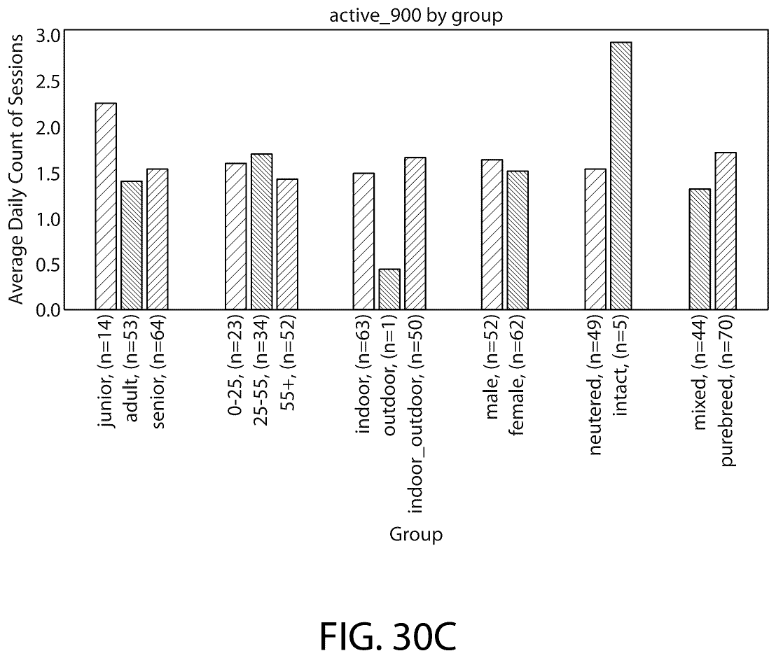

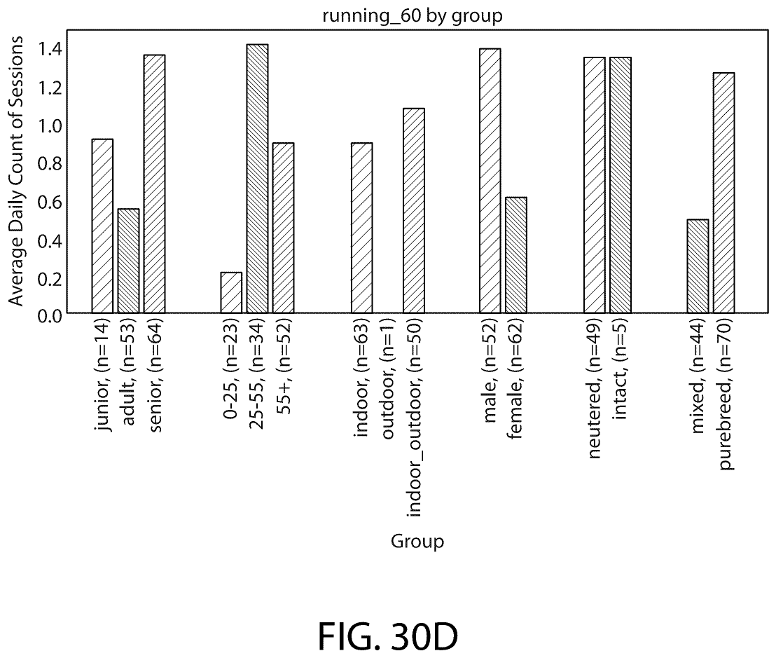

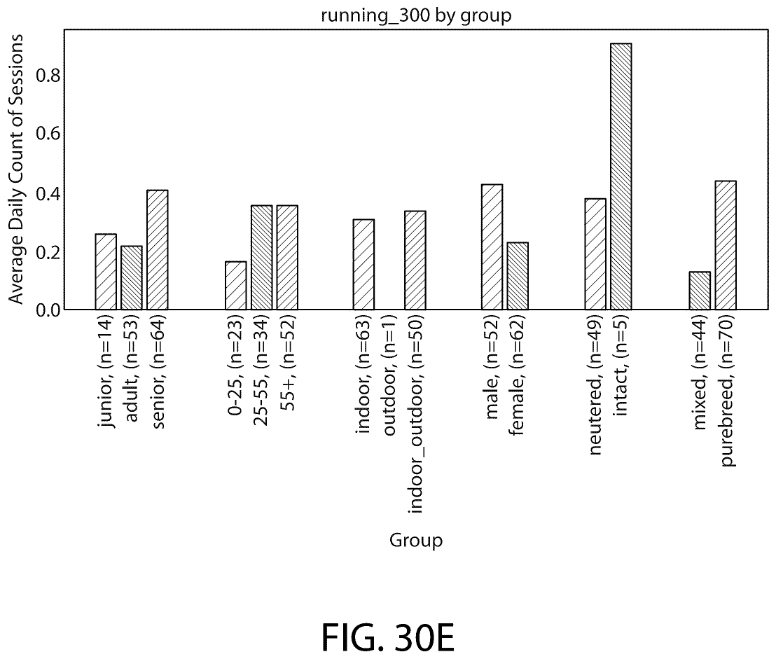

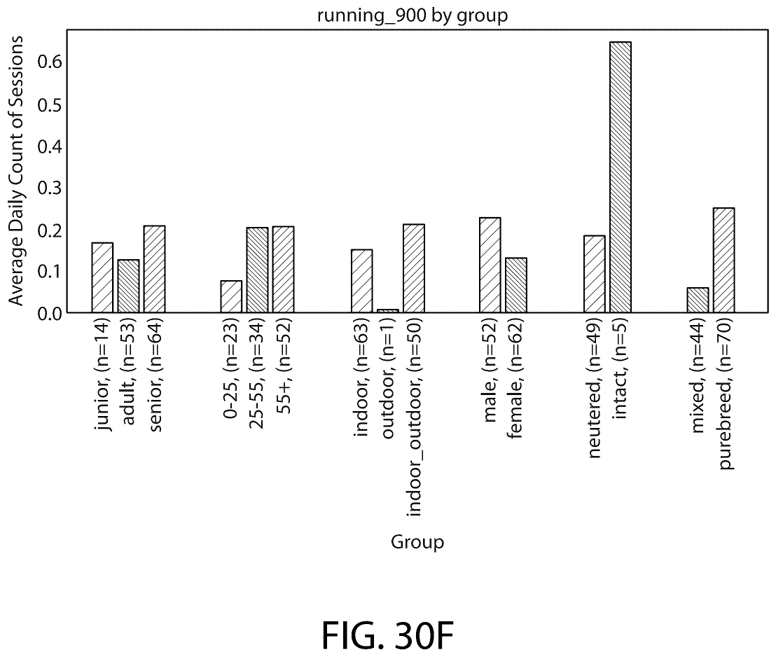

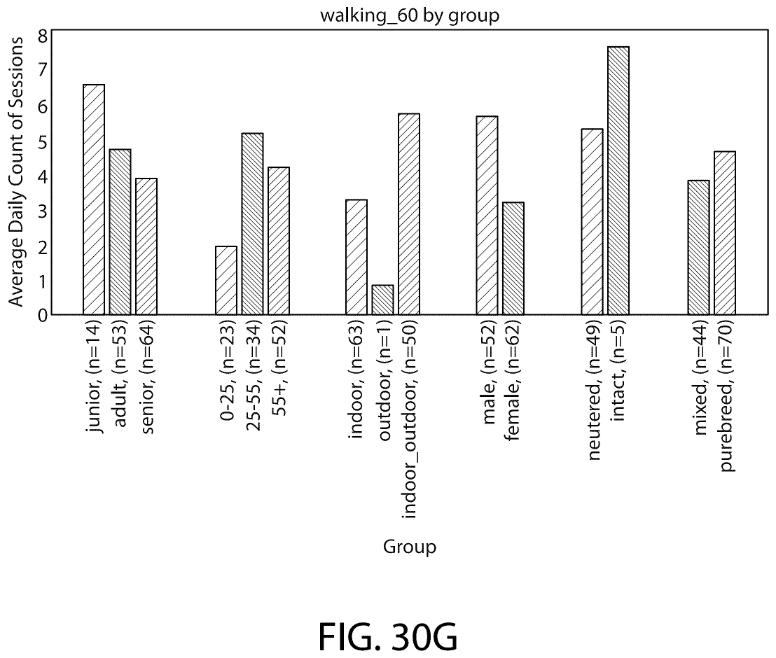

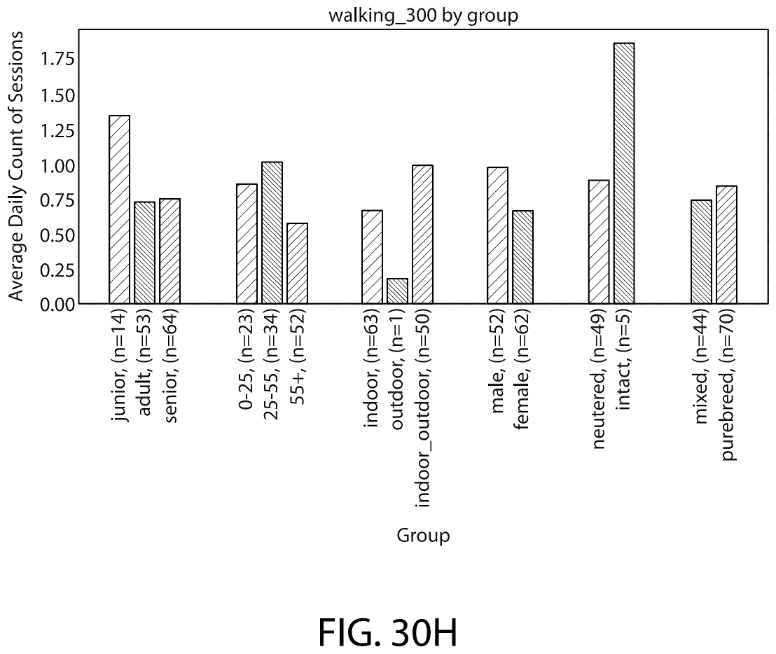

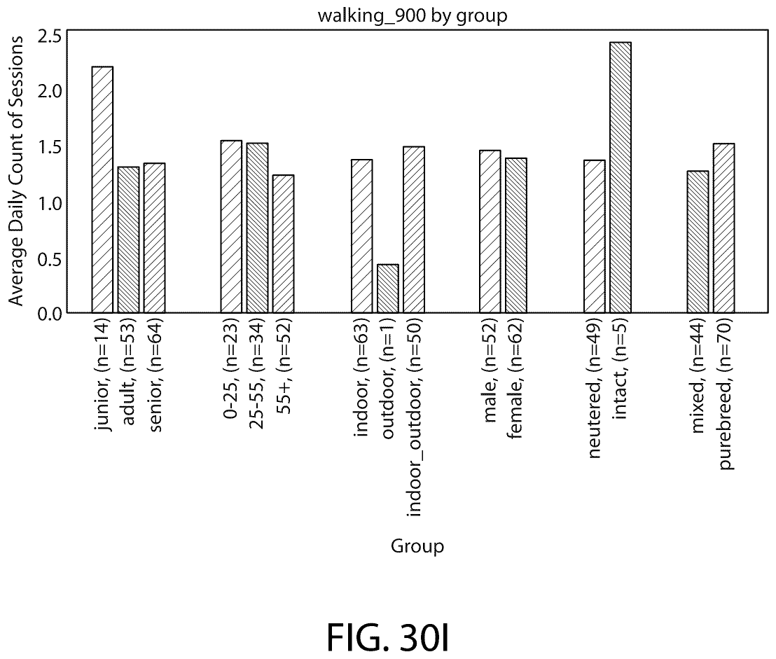

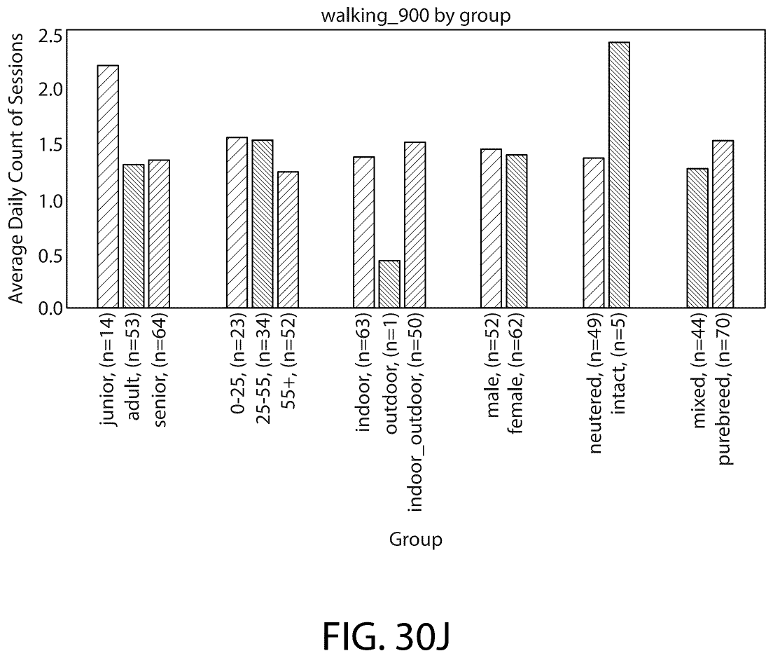

FIG. 30A through 30J depict sessionization data of groups of pets. The bar charts show differences in session exhibition between different subgroups.

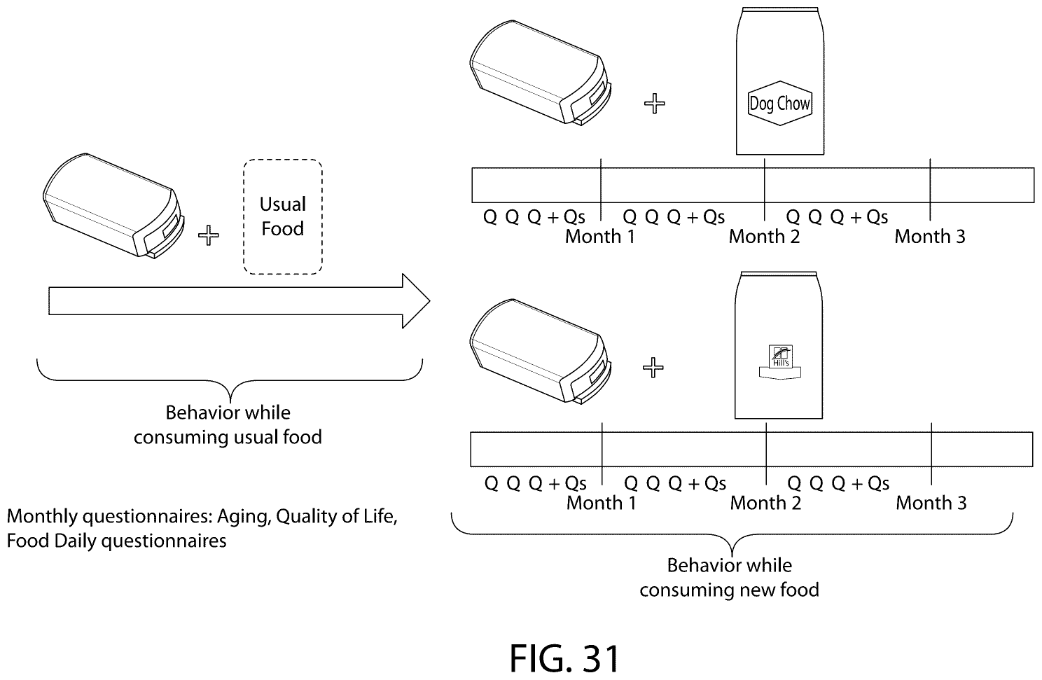

FIG. 31 depicts a study design associated with a dog experiencing a diet change. Embodiments comprise methods along any study design herein and systems incorporating devices to harvest data for use in the methods. As illustrated in FIG. 31, a first portion of the study comprises monitoring behavior while the dog is consuming its usual food. A second portion comprises monitoring behavior while the dog is consuming new food. The study may implement at least one of a wearable sensor herein, a smart toy herein, a smart bed herein, or a smart room herein in order to monitor the behavior and harvest data related to the same. The study could also incorporate cameras for the ingestive behavior measures; for example, those described above. The study could also incorporate BFI, BCS, Weight, etc. estimations; for example, those described above. There are many different permutations that could be envisioned. The algorithms described above could be used, etc. The system may also implement an algorithm herein to provide displays of the data and/or conclusions based on the data. The design may include periodic; e.g., monthly, questionnaires filled out be a person observing the dog; e.g., its owner.

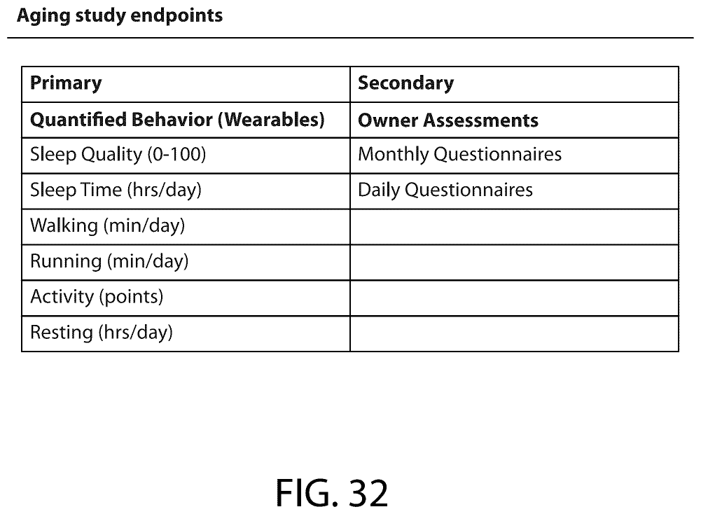

FIG. 32 depicts a set of study design endpoints. Primary endpoints may be quantified behavior. The primary endpoints may be data harvested by a wearable herein. Primary endpoints may be data harvested by at least one of a wearable sensor herein, a smart toy herein, a smart bed herein, or a smart room herein. Study design endpoints may be the endpoints of study design as described above for FIG. 31.

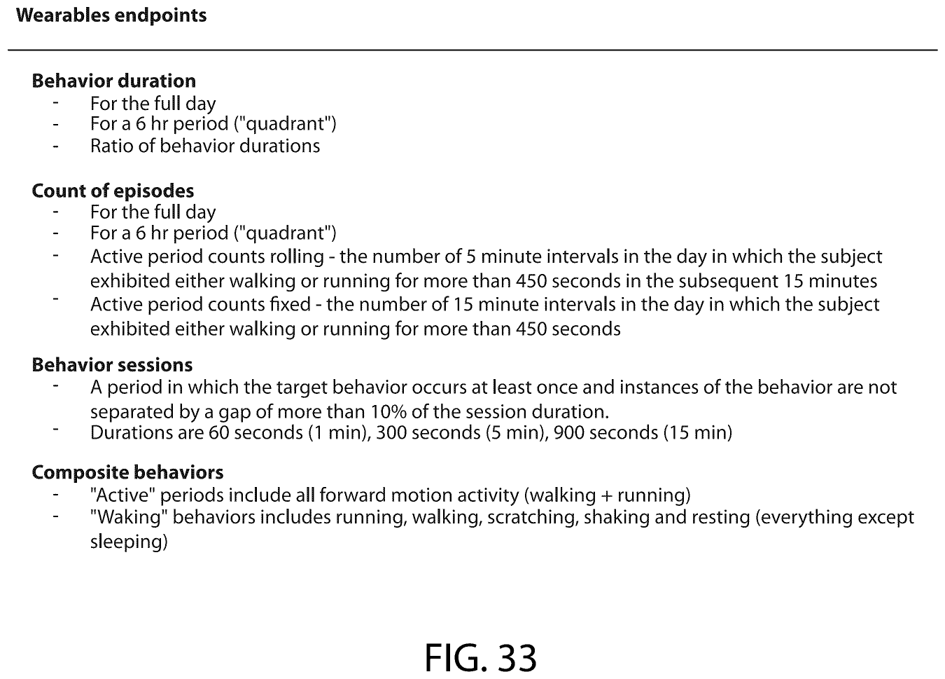

FIG. 33 depicts a set of metrics measured on a wearable sensor. The metrics may include behavior duration, count of episodes, behavior sessions, or composite behaviors. The behavior duration may be for a full day, for 6 hour period (or a “quadrant”), or a ratio of behavior durations. The counts of episodes may occur For a full day, for a 6 hr. period (“quadrant”), active period counts rolling—the number of 5 minute intervals in the day in which the subject exhibited either walking or running for more than 450 seconds in the subsequent 15 minutes, active period counts fixed—the number of 15 minute intervals in the day in which the subject exhibited either walking or running for more than 150 seconds. The behavior session may include a period in which the target behavior occurs at least once and instances of the behavior are not separated by a gap of more than 10% of the session duration. Durations may be 60 seconds (1 min), 300 seconds (5 min), or 900 seconds (15 min). Composite behaviors may involve “Active” periods that include all forward motion activity (walking+running) or “Waking” behaviors including running, walking, scratching, shaking and resting (everything except sleeping).

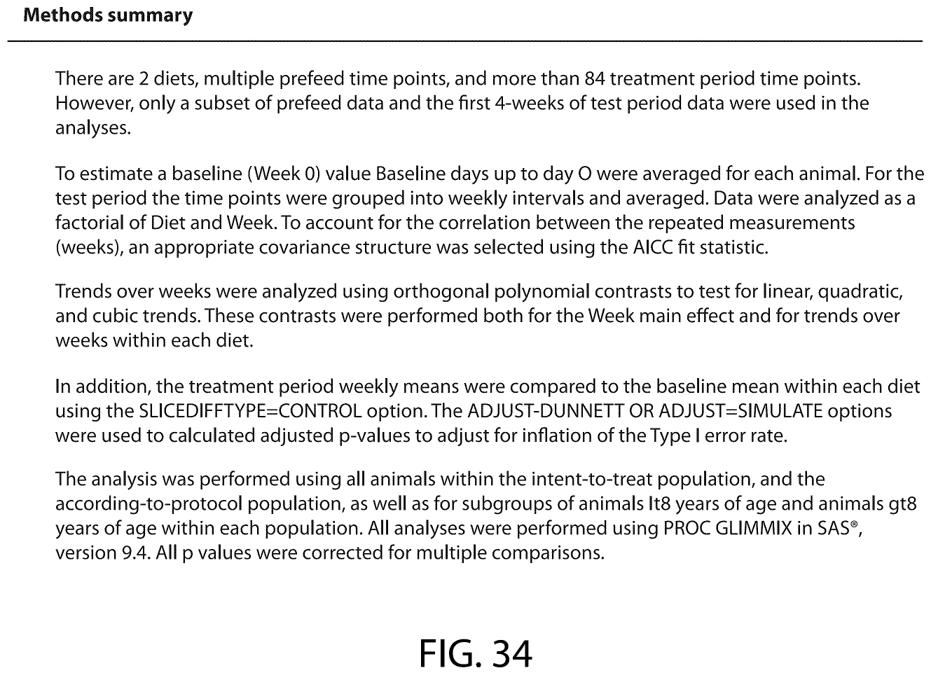

FIG. 34 depicts a methods summary of a study design including a wearable sensor on a subject. In this study, there were 2 diets, multiple prefeed time points, and more than 84 treatment period time points. However, only a subset of prefeed data and the first 4-weeks of test period data were used in the analyses. To estimate a baseline (Week O) value Baseline days up to day O were averaged for each animal. For the test period the time points were grouped into weekly intervals and averaged. Data were analyzed as a factorial of Diet and Week. To account for the correlation between the repeated measurements (weeks). an appropriate covariance structure was selected using the AICC fit statistic. Trends over weeks were analyzed using orthogonal polynomial contrasts to test for linear, quadratic, and cubic trends. These contrasts were performed both for the Week main effect and for trends over weeks within each diet. In addition, the treatment period weekly means were compared to the baseline mean within each diet using the SLICEDIFFTYPE=CONTROL option. The ADJUST=DUNNETT OR ADJUST=SIMULATE options were used to calculated adjusted p-values to adjust for inflation of the Type I error rate. The analysis was performed using all animals within the intent-to-treat population, and the according-to-protocol population, as well as for subgroups of animals lt8 years of age and animals gt8 years of age within each population. All analyses were performed using PROC GLIMMIX in SAS®, version 9.4. All p values were corrected for multiple comparisons.

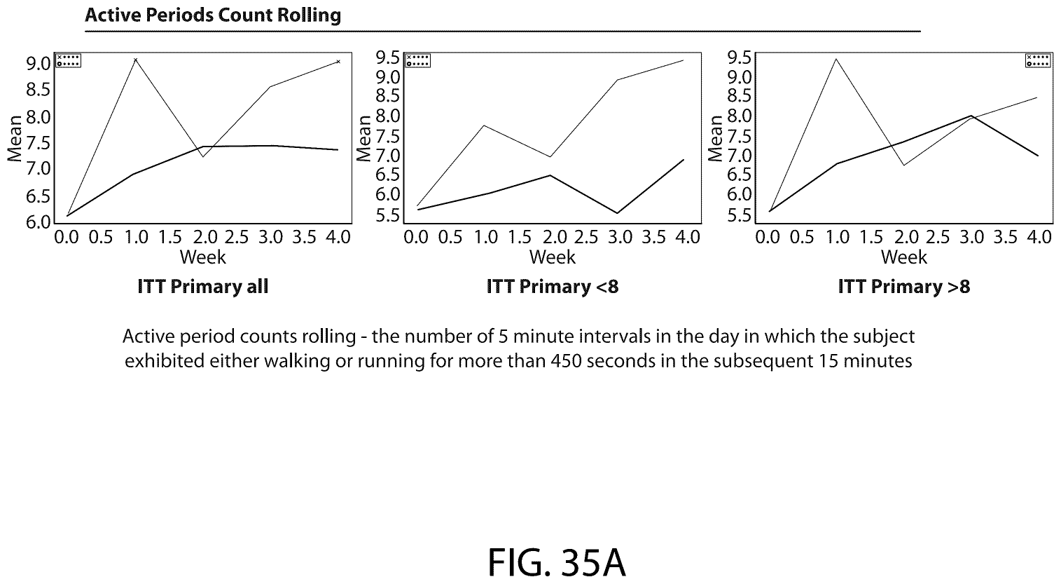

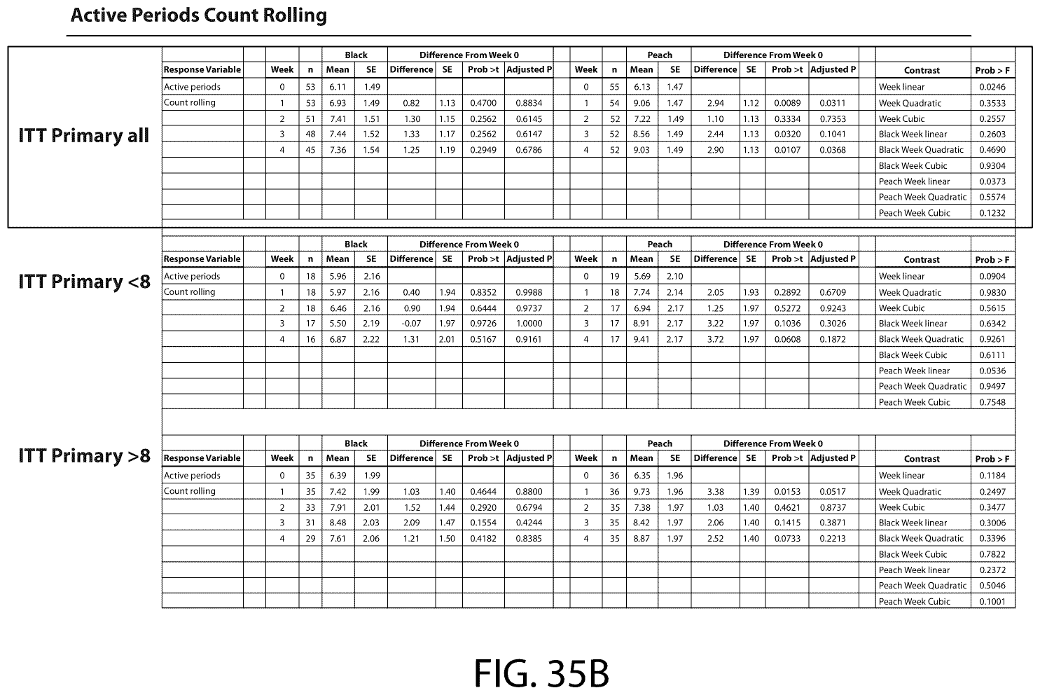

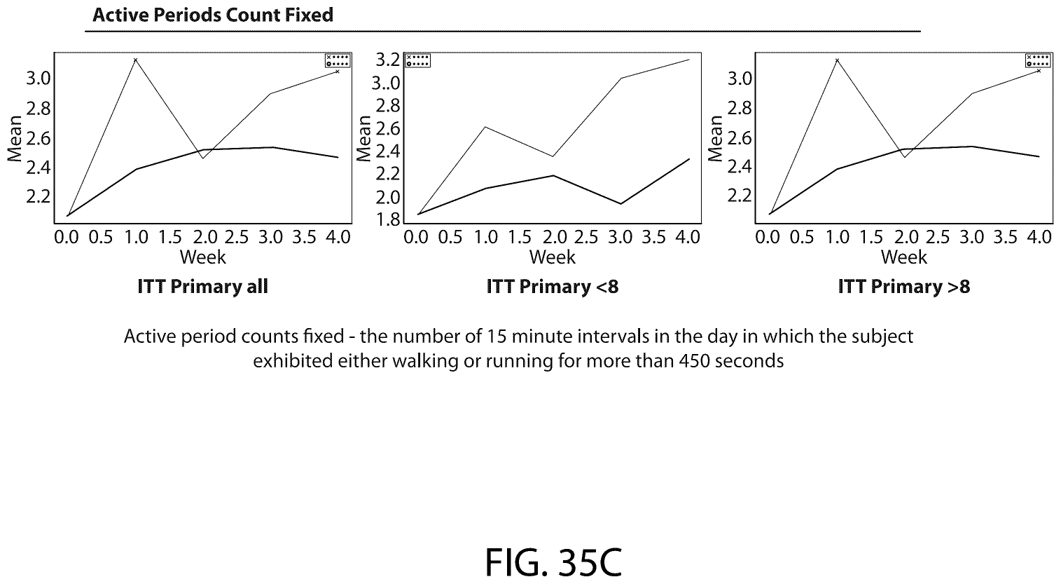

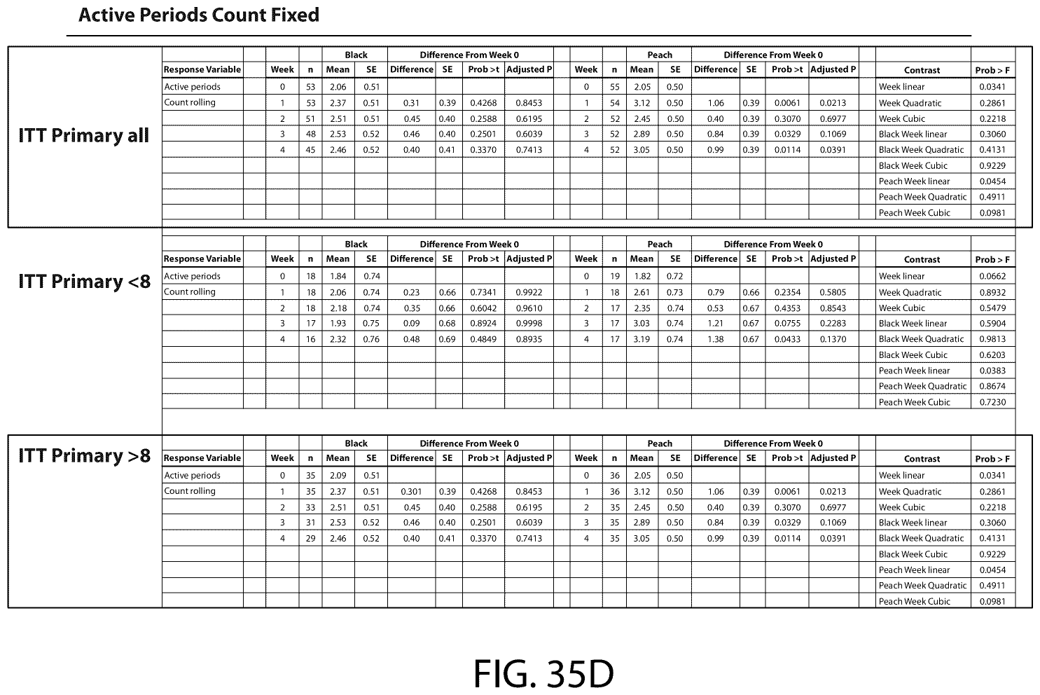

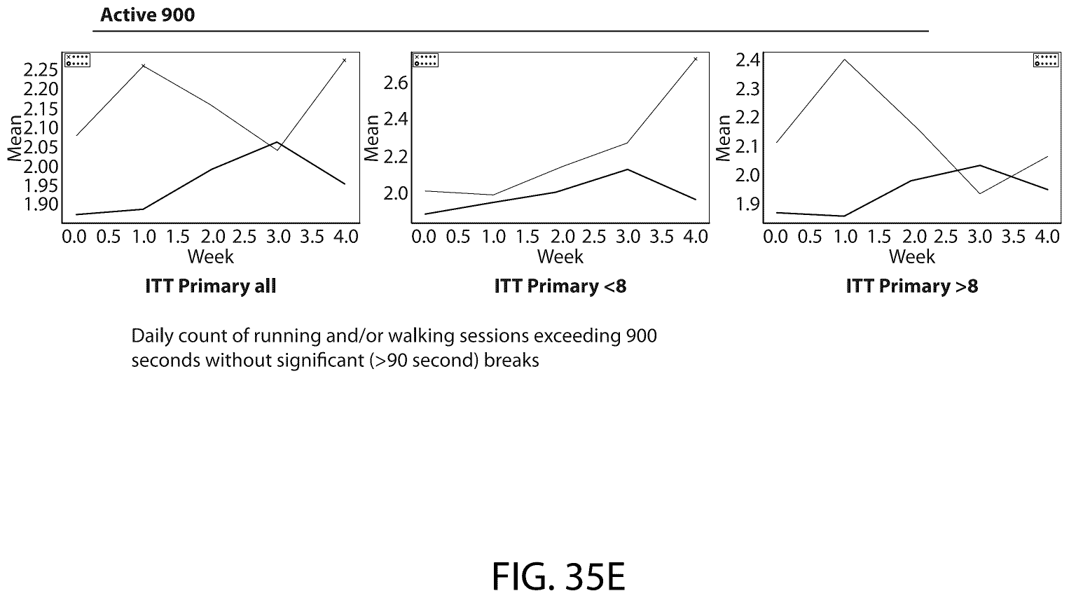

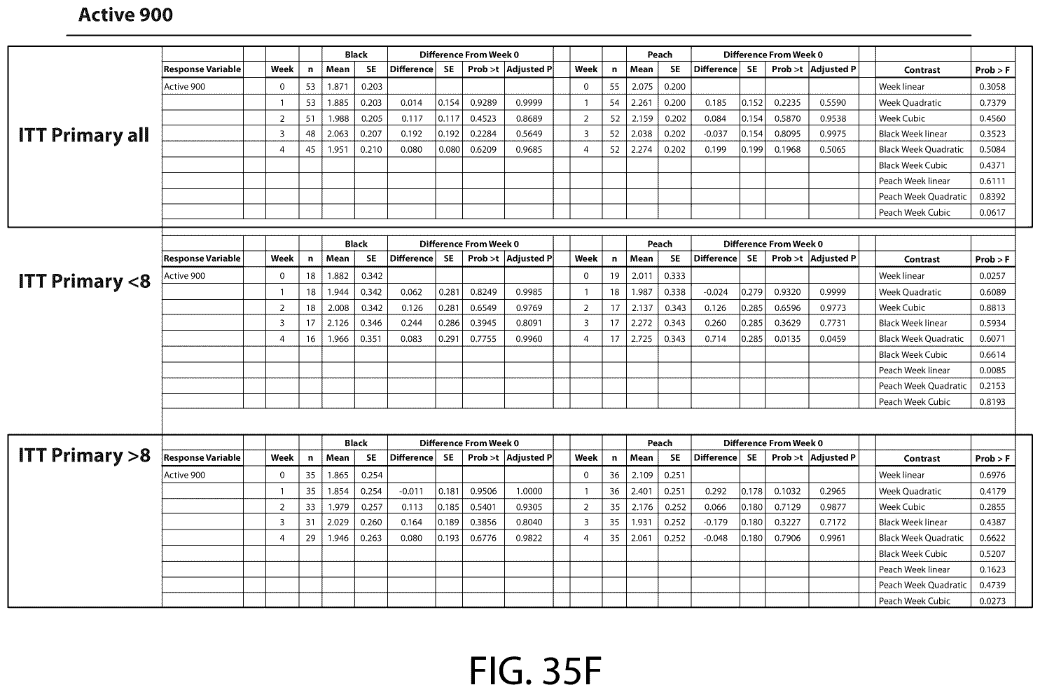

FIG. 35A through 35G depict a set of data related to activity measurements of pets.

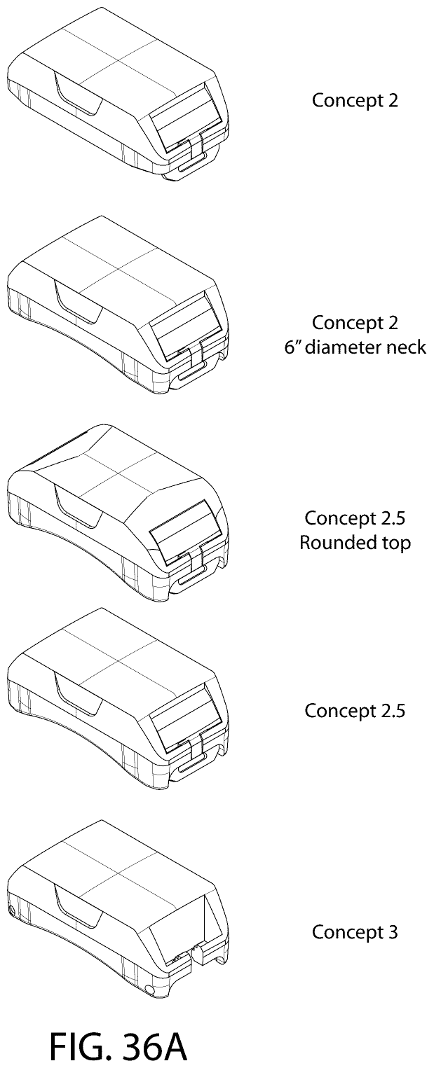

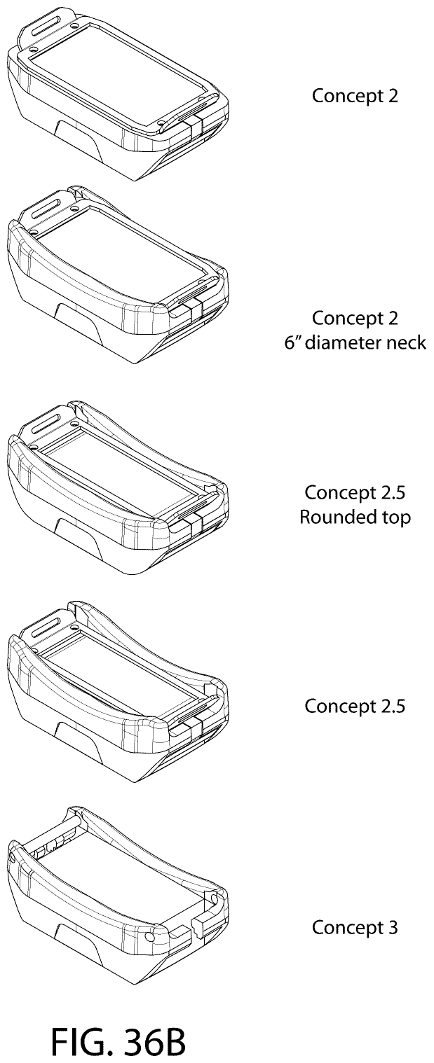

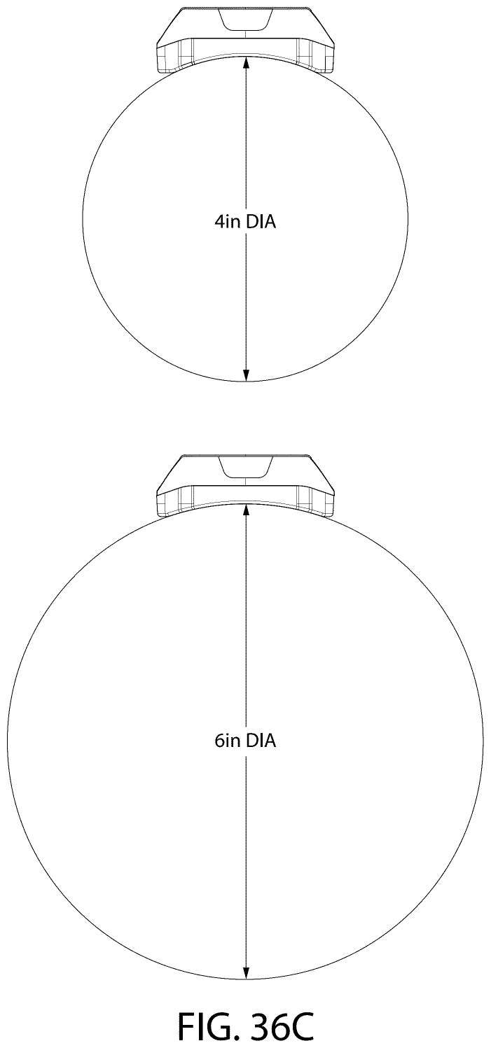

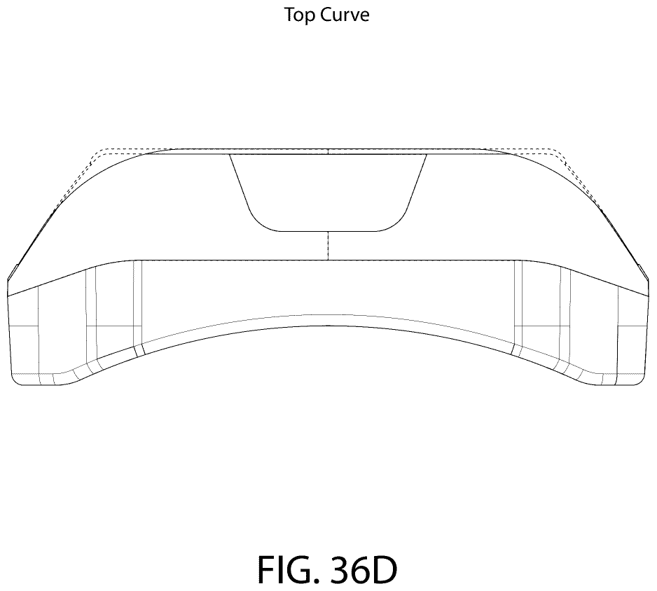

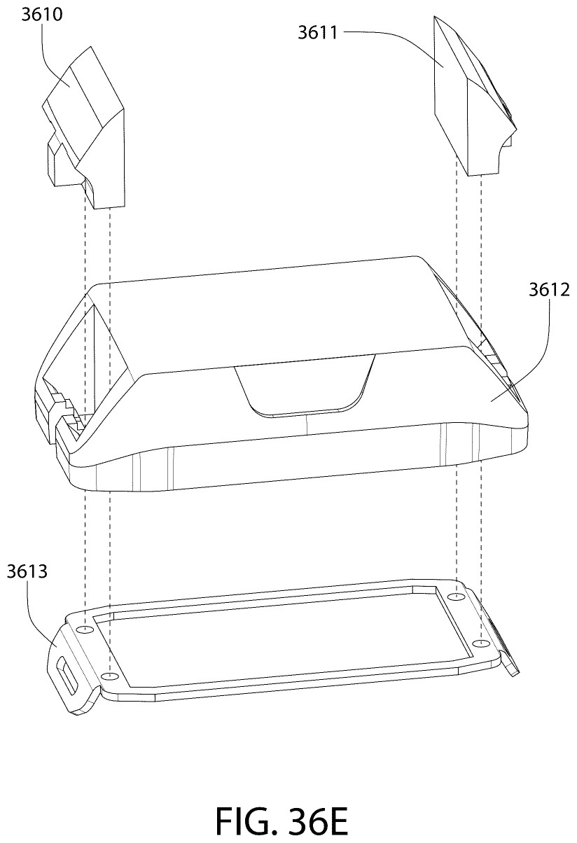

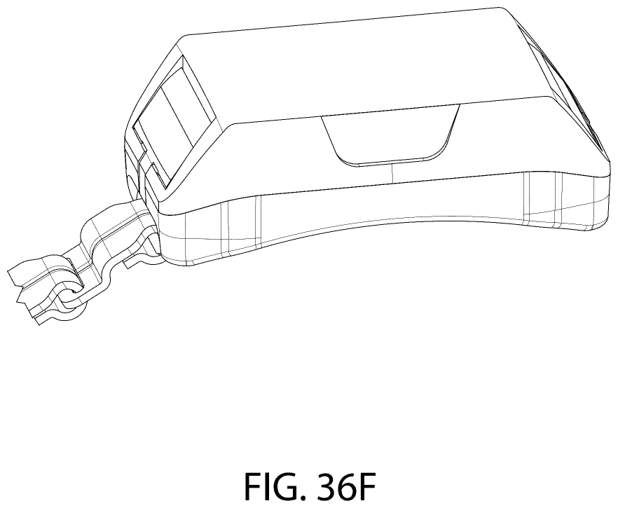

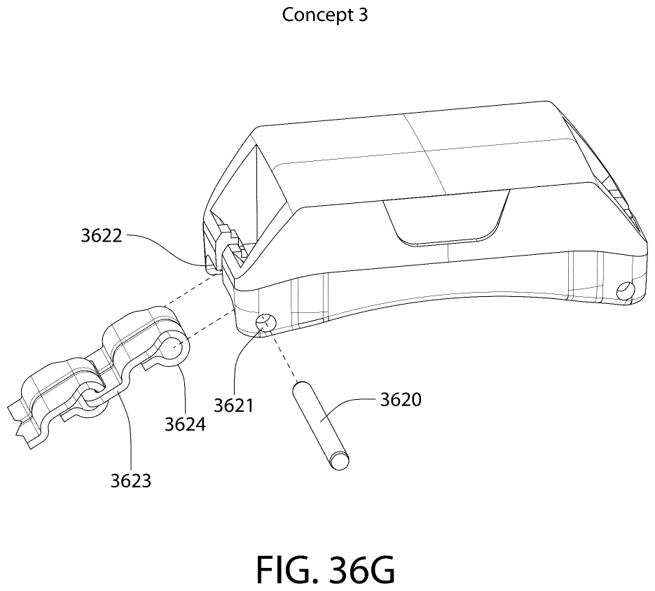

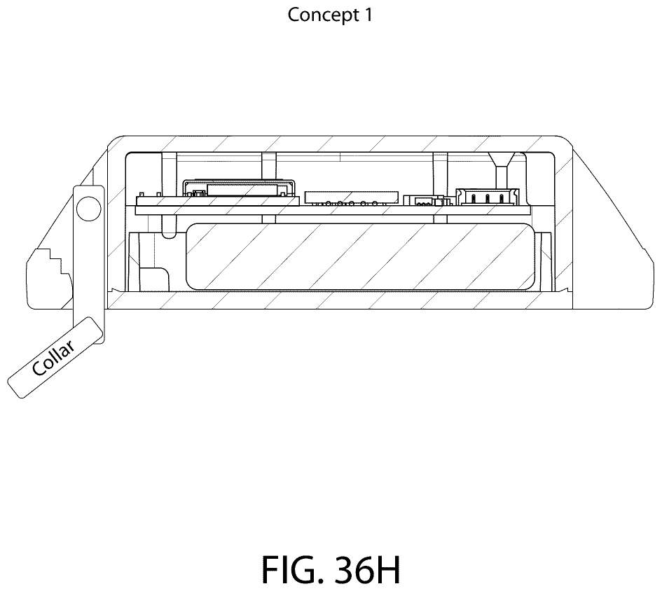

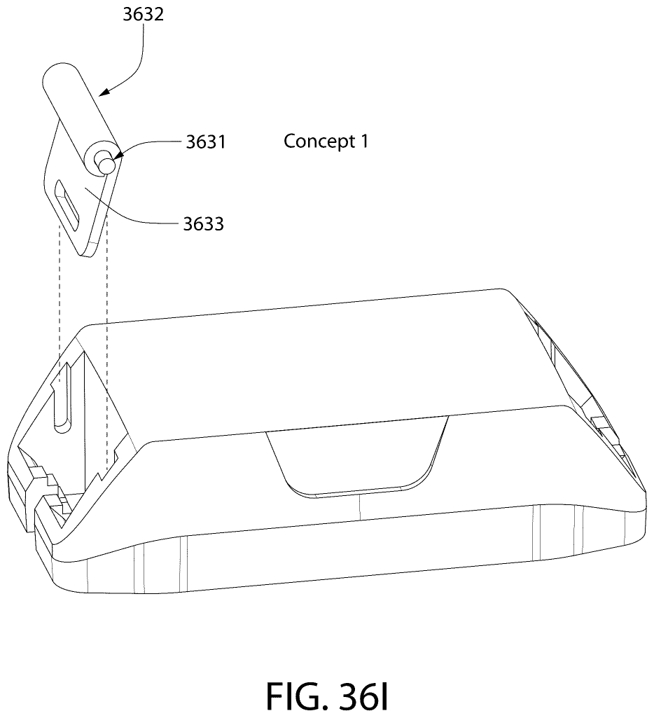

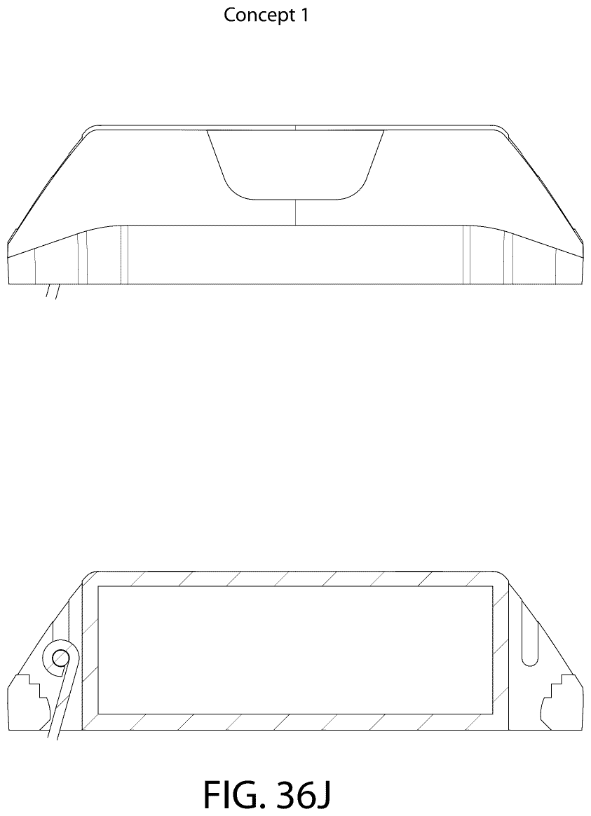

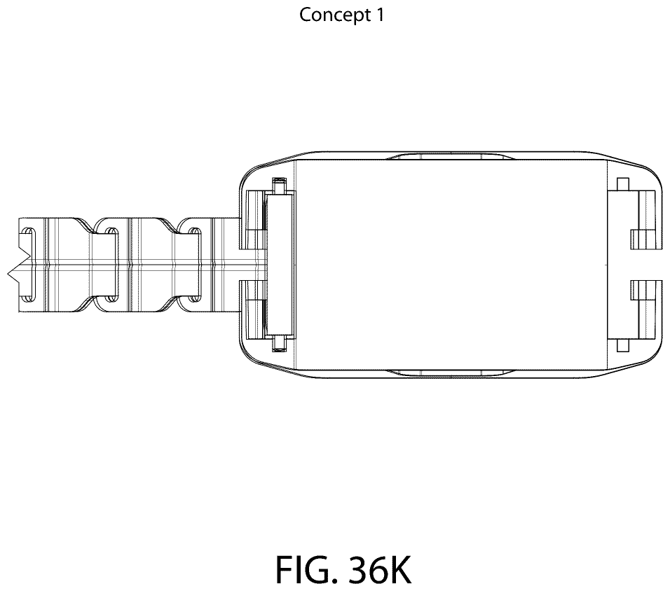

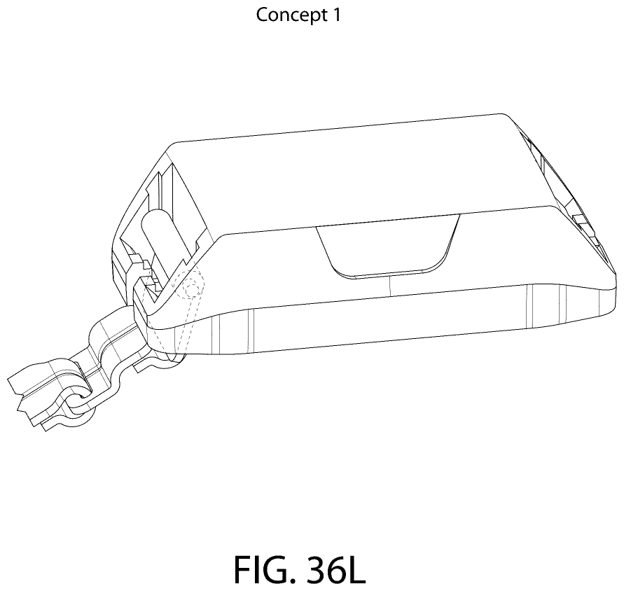

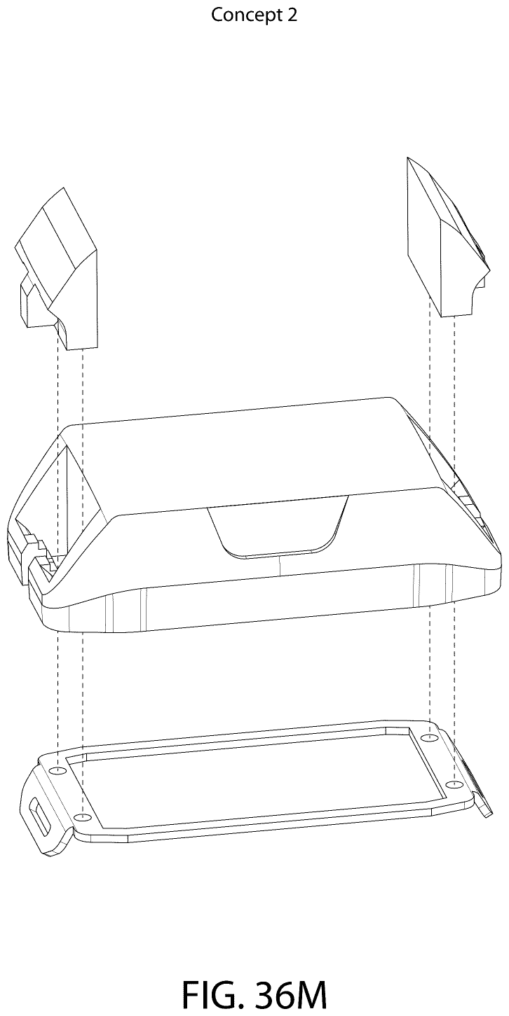

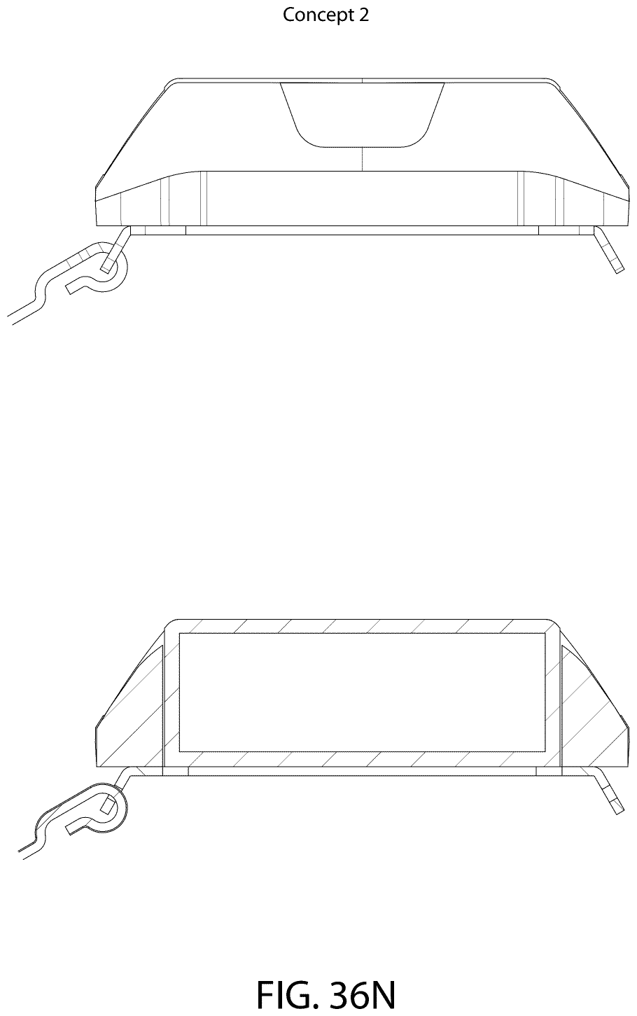

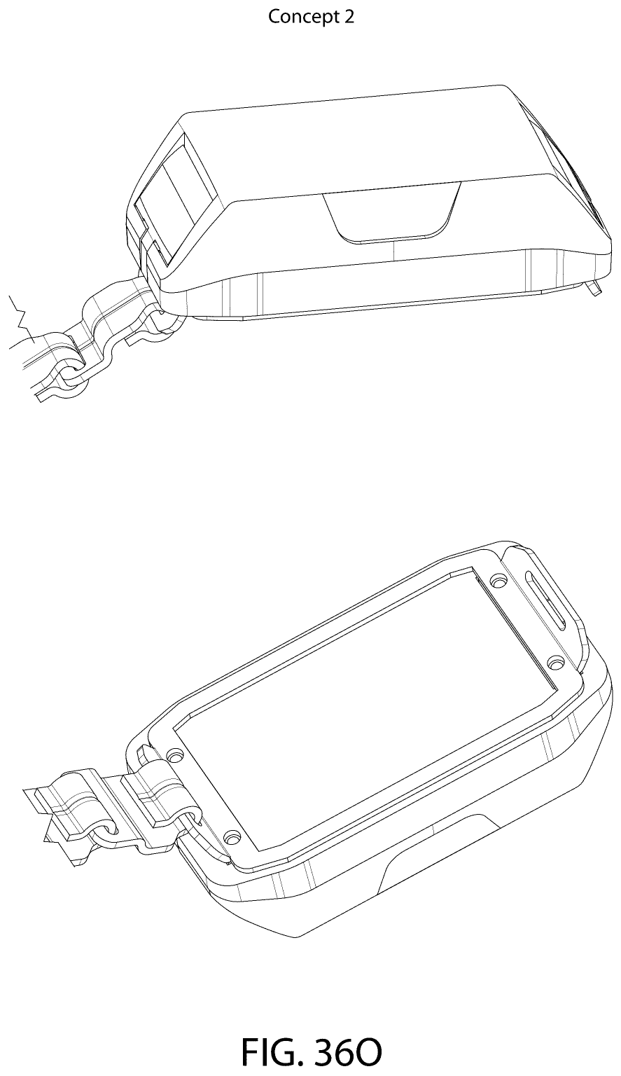

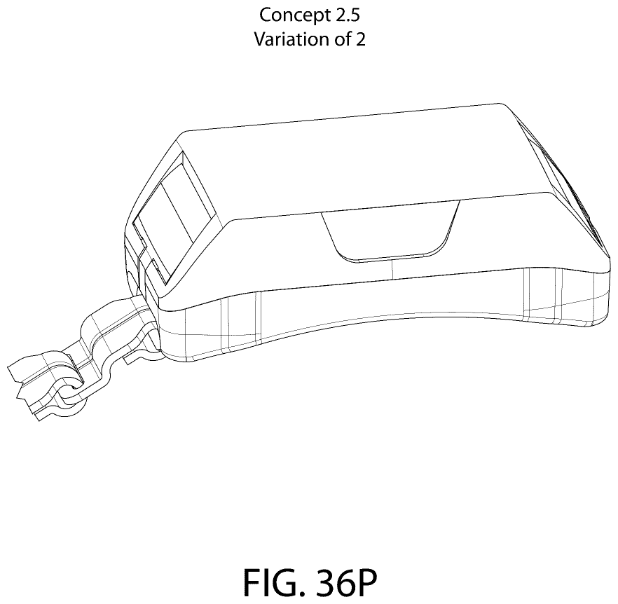

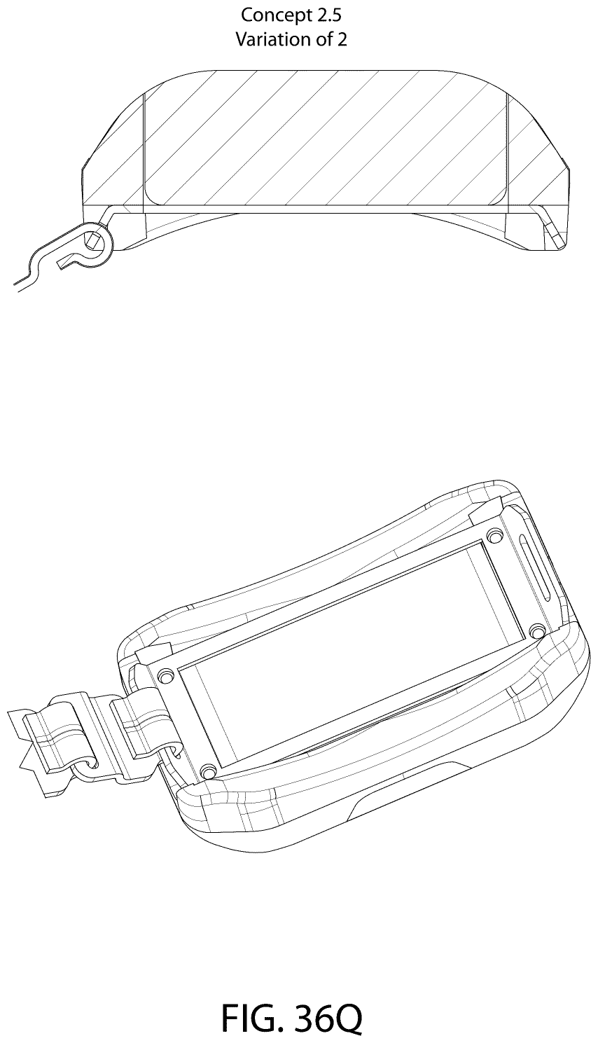

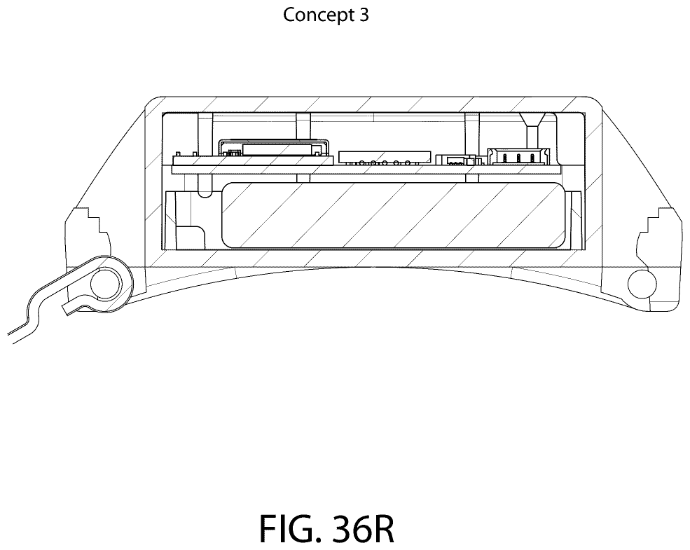

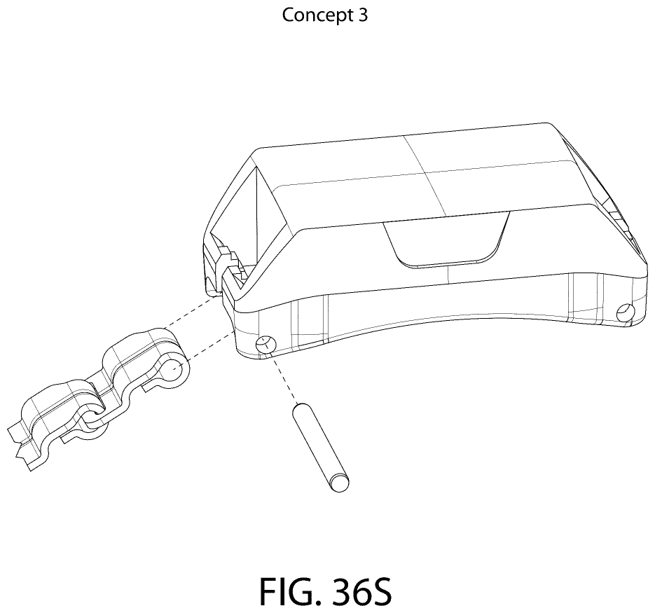

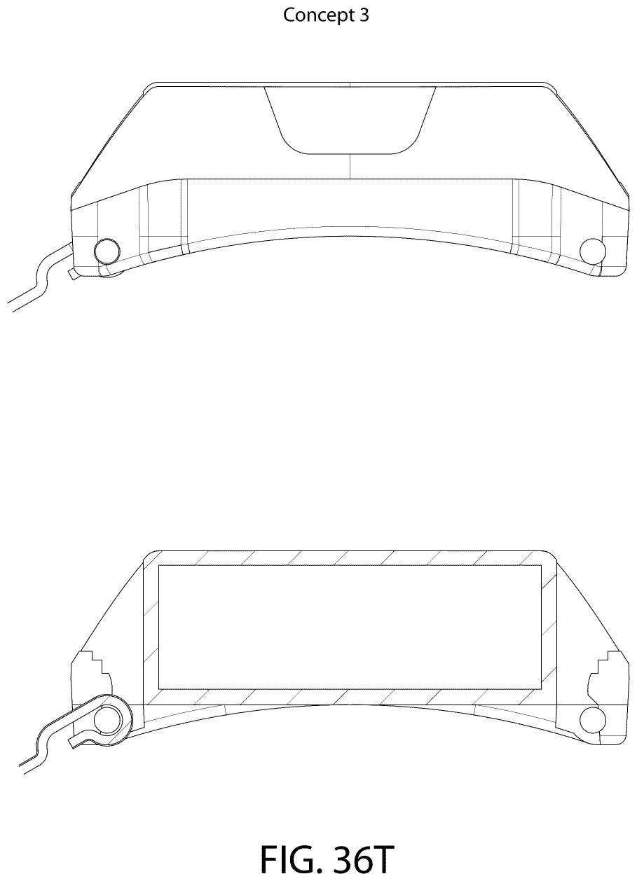

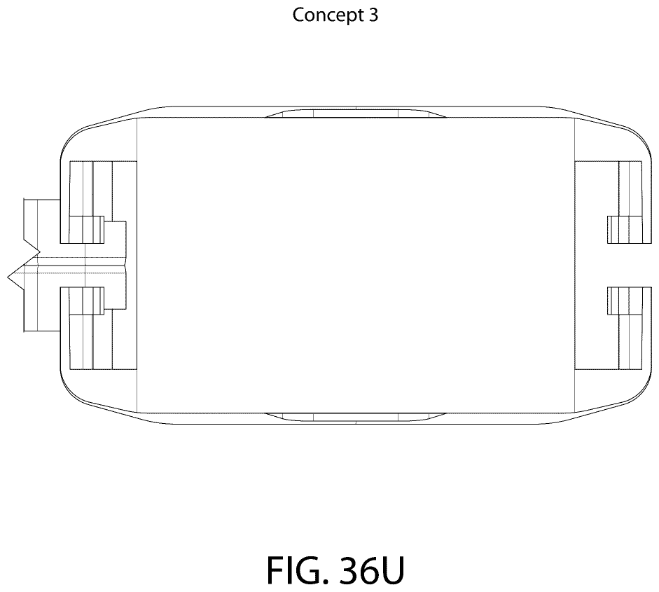

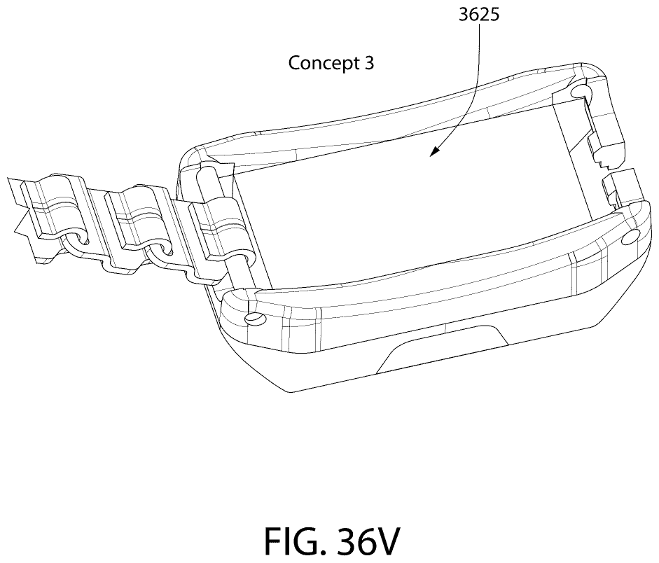

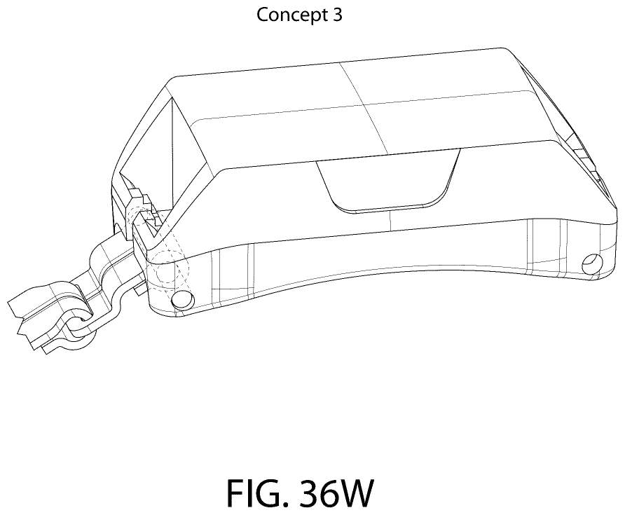

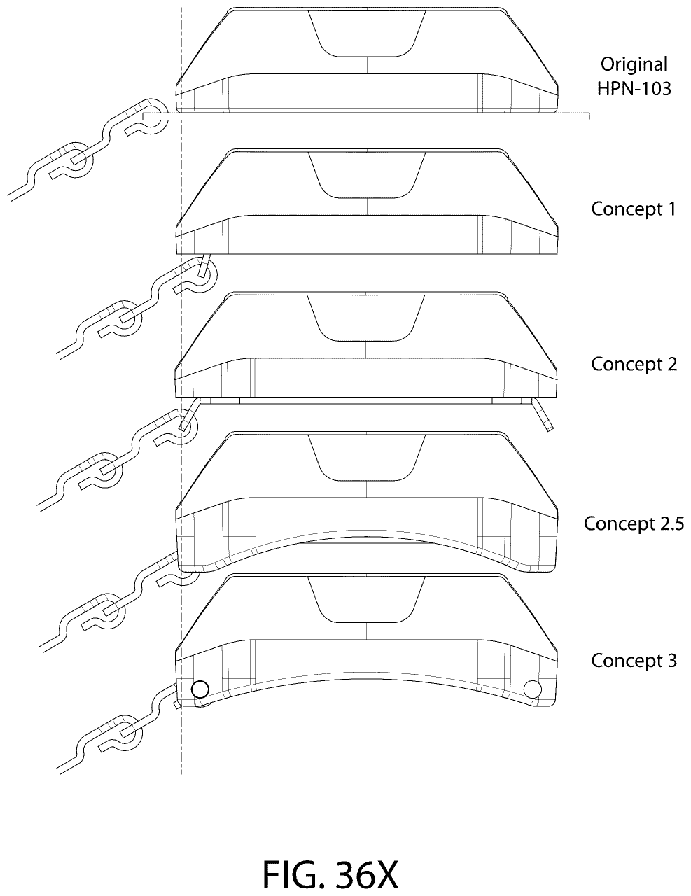

FIG. 36A through 36X depict embodiments of a wearable sensor incorporated in a collar for a domesticated animal. The sensors may include the basic elements as described above for a sensor. FIGS. 36A and 36B align various concepts for the wearable sensors.

FIG. 36 C illustrates wearable sensors designed for pets with different neck sizes. The device on the left is labeled for a four inch neck diameter, while the device on the right is labeled for a six inch neck diameter. However, embodiments herein comprise wearable sensors having a collar sufficient for any neck size. Further embodiments comprise wearable sensors with adjustable collars that may be adapted to the neck size of the animal upon which it is applied.

FIG. 36D illustrates a biosensor with trimmed corners. The trimmed corner portion is the dark shaded portion at the most sharply angled parts of the top sensor surface. As view at the upper right and upper left corners of the sensor. An embodiment is to trim the corners to soften the overall profile of the sensor, and remove the sharpest angles on the top surface. Portions that may be candidates for trimming are the portions shown in the darkest gray shading.

FIGS. 36E and 36M though 36O illustrates a wearable sensor according to concept 2, as labeled in FIGS. 36A and 36B. comprising a first housing 3610 and a housing 3611. One or both of the first and second housing may be plastic. As illustrated, the first housing 3610 and the second housing 3611 may slide into position on a main housing 3612. The main housing may be plastic. Also illustrated in FIG. 36E is a bottom plate 3613 that fits to the bottom of the main housing 3612. The bottom plate may be aluminum. Fasteners may be extended through the plate 3613 and the main housing 3612 and into the first and second housing 3610, 3611. One or both of the first and second housing 3610, 3611, also referred to as first and second plugs, may be included to cover strap lugs, for example nylon strap lugs. The plug may be used as a location to place or encode information regarding the animal. For example, medications may be indicated by name or different colors, the animals name could be provided thereon.

FIGS. 36F, 36P, and 36Q illustrate a wearable sensor according to concept 2.5, a variation of concept 2, as labeled in FIGS. 36 A and 36B. The concept 2.5 device is similar to the concept 2 device, but has a “concept 3” housing. The housing is extended downward on the ends. This may help keep the bottom plate from being exposed, and may better contour to the neck of the animal; e.g., a cat or dog, on which it is worn.

FIGS. 36G and 36R through 36W illustrate a wearable sensor according to concept 3 as labeled in FIGS. 36A and 36B. Concept 3 is similar to concept 2.5 with the housing extending downward and additional comprises a dowl pin 3620 and respective pin receivers 3621 and 3622 in the housing. The dowel pin 3620 may be used to engage a collar 3623. When engaged with the collar 3623 and the pin receivers 3621, 3622. In an embodiment, the collar is a metal collar comprising links where the last link 3624 engages the dowel pin 3620. A strap of a collar may lie against a back 3625 as illustrated in FIG. 36V.

FIGS. 36H, 36I, 36J, 36K, 36L illustrate a wearable sensor according to concept 1. The general features are as described above for concept 2, with the following differences. A dowel pin 3631 is included in the first housing 3632 to engage with grooves on the main housing. The first housing includes a slot 3633 that may engage with a collar. The first and second housing may be metal in order to provide strength in the collar engagement. As illustrated in FIGS. 36J through 36L, a collar may engage the slot.

FIG. 36 X compares the wearable sensor of an HPN-103 wearable sensor above the concept 1, concept 2, concept 2.5, and concept 3 variations, from top to bottom. The comparison highlights the centerline of the first link on the collar for each of the concepts.

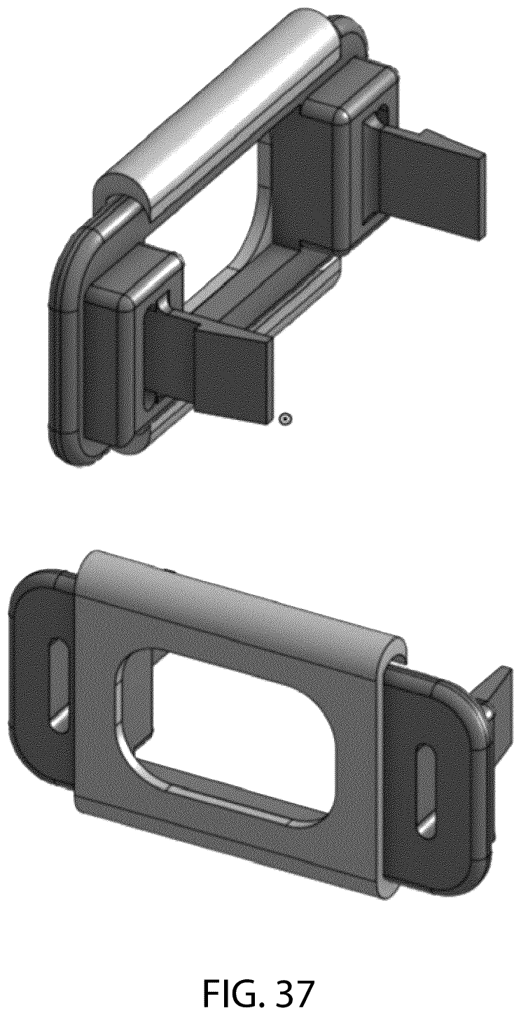

FIG. 37 depicts a modified embodiment for a wearable sensor incorporated in a collar for a pet with an HPN1 attachment. The c-clip design is to hold HPN1 with a snap friction fit.

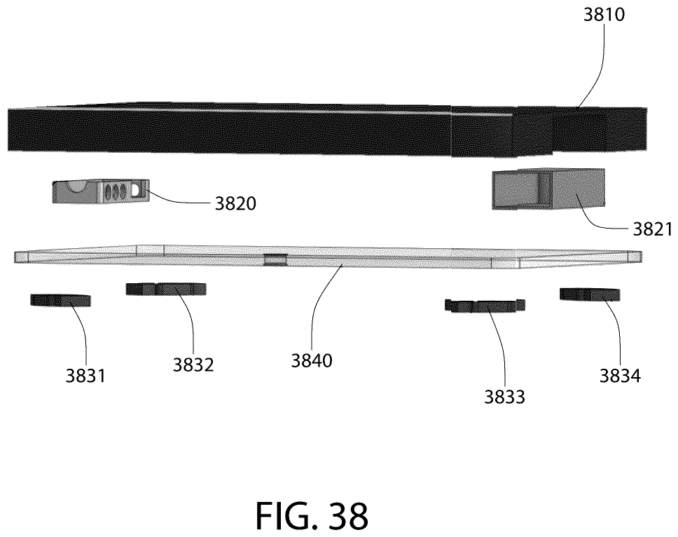

FIG. 38 depicts a design smart bed comprising a sensor incorporated in a bedding of a pet. The smart bed comprises a pad 3810, which may be foam, battery holders 3820, 3821, load cell housings 3831, 3832, 3833, and 3834, and a weight frame 3840, which may be acrylic.

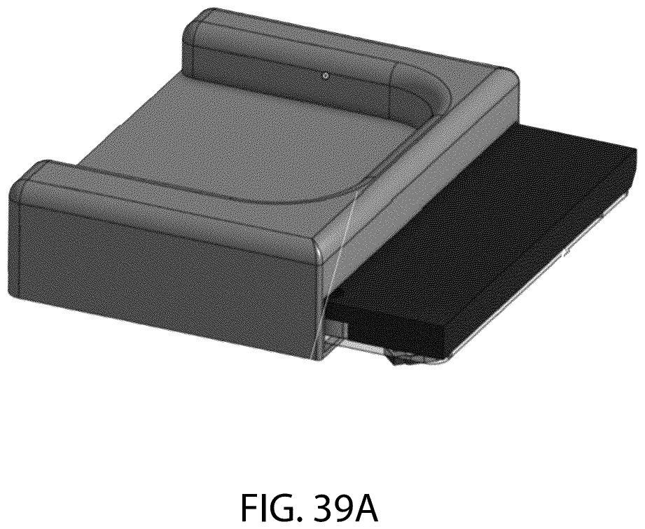

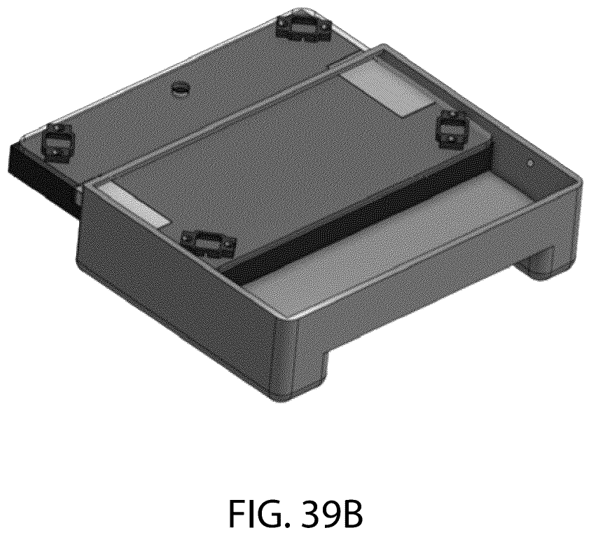

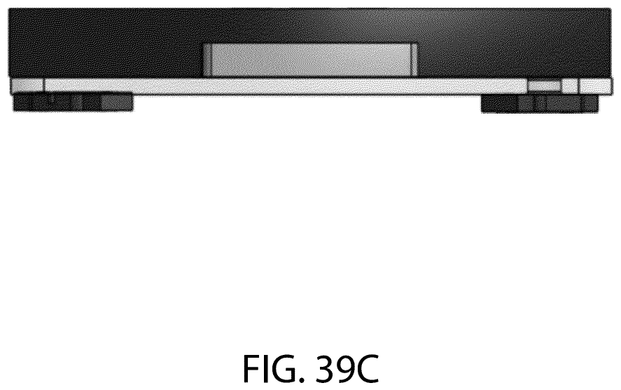

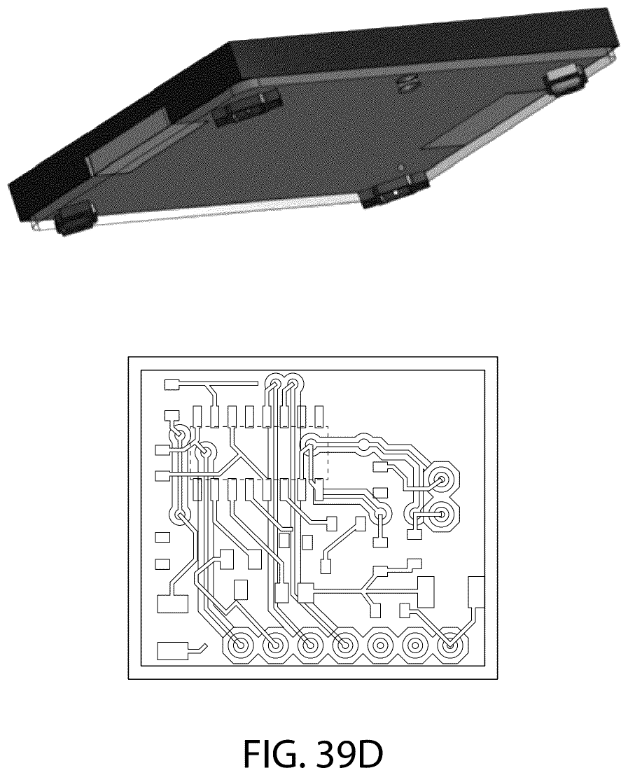

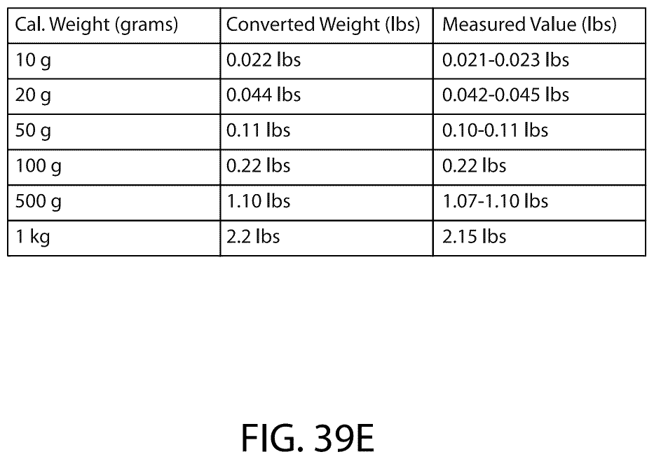

FIG. 39A through 39E depict embodiments of the sensor incorporated within bedding for a pet. FIG. 39A illustrate a top view of the smart bed with the weight frame being slid underneath, which FIG. 39B illustrates the bottom view. FIG. 39C illustrates a side view. The smart bed in this configuration may be smaller. It may have a footprint of about 24 inches by about 18 inches by about 3 inches (including two inches of foam). It may fit within the outer casing of store-bought beds. It may weigh about 12 pounds. FIG. 39D illustrates a weight frame having 4 half-bridge load cells as leges of the device. The legs may take the weight of the animal. The load cells may be connected to an Hx711 analog-to-digital converter/multiplexer that can be connected to an MCU. Wires may be routed underneath the frame and through a hole under the foam, and may be fully insulated. The weight frame may be acrylic. FIG. 39D illustrate weight frame calibration with calibrated weights. The calibration constant was 5000.

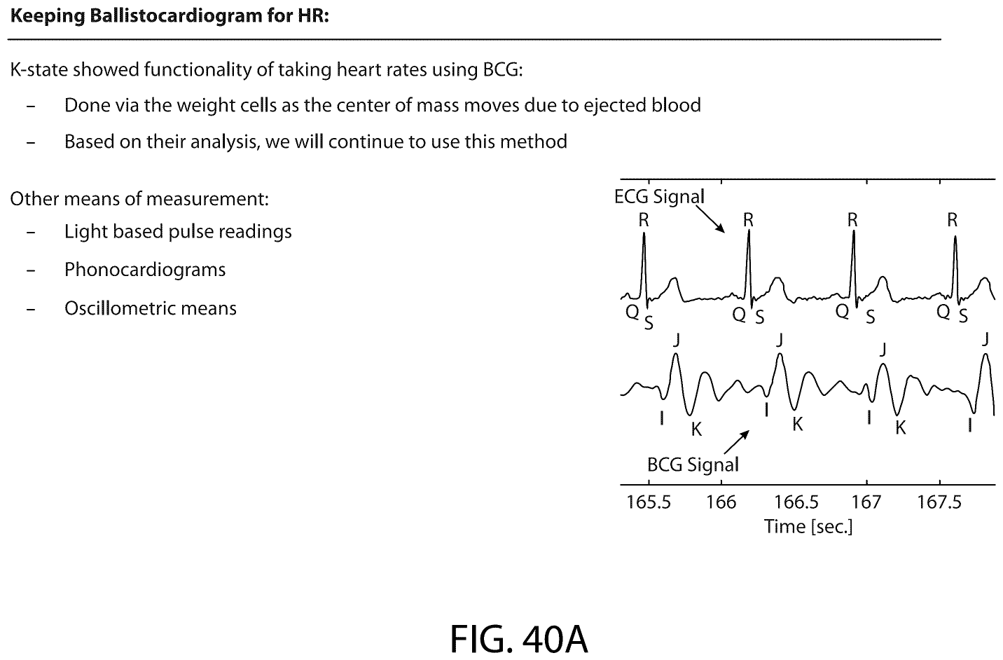

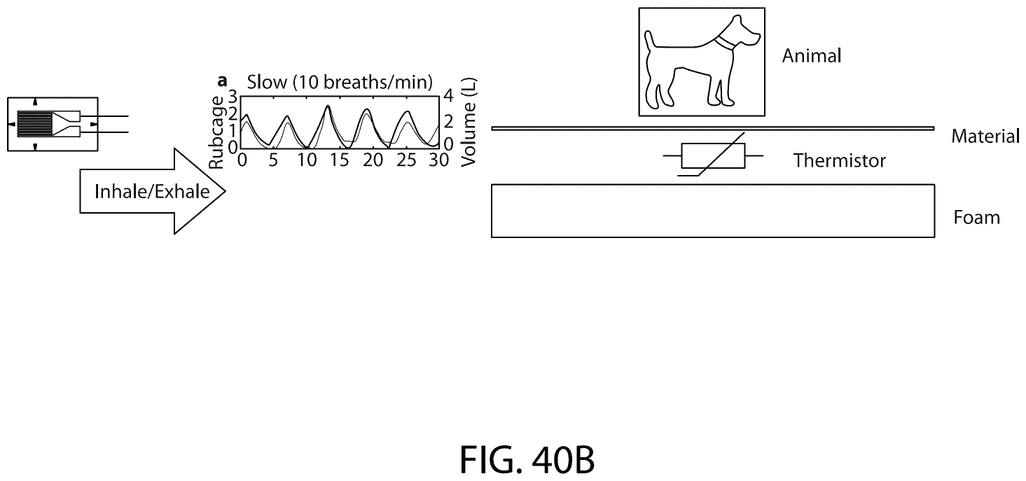

FIGS. 40A through 40C depict a collection of metrics to be measured with embodiments of the sensor incorporated within bedding for a pet. FIG. 40A illustrates functionality of taking heart rates using BCG. This was done via the weight cells as the center of mass moves due to ejected blood. Other means of measurement may include light based pulse readings, phonocardiograms, and oscillometric means. FIG. 40B illustrates functionality that may be added to a design herein. Respiration rate may be monitored. The design may include the whealston bridge system to measure changes in abdominal volume as an animal inhales and exhales on the bed. Temperature may also be monitored. Embodiments comprise a thermistor set-up to measure contact temperature when the animal lays on the bed.

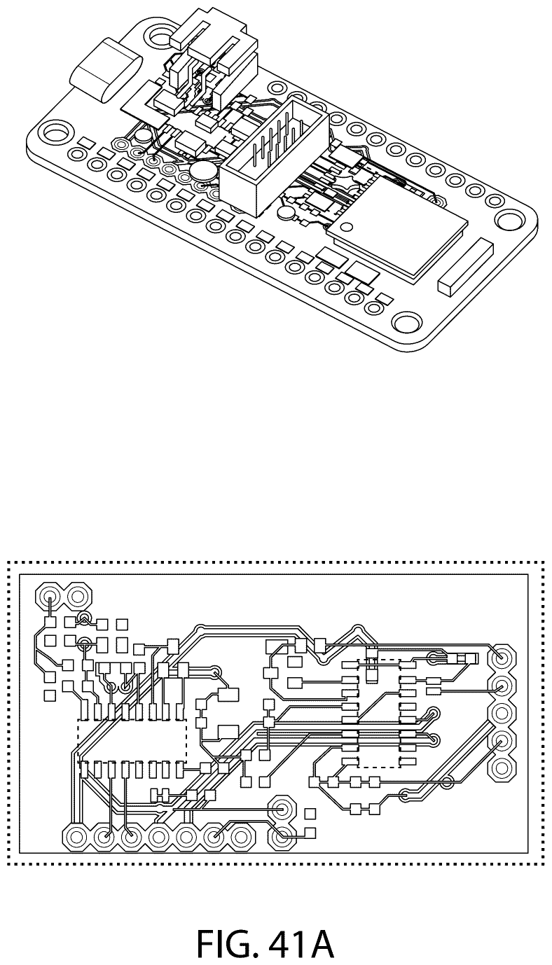

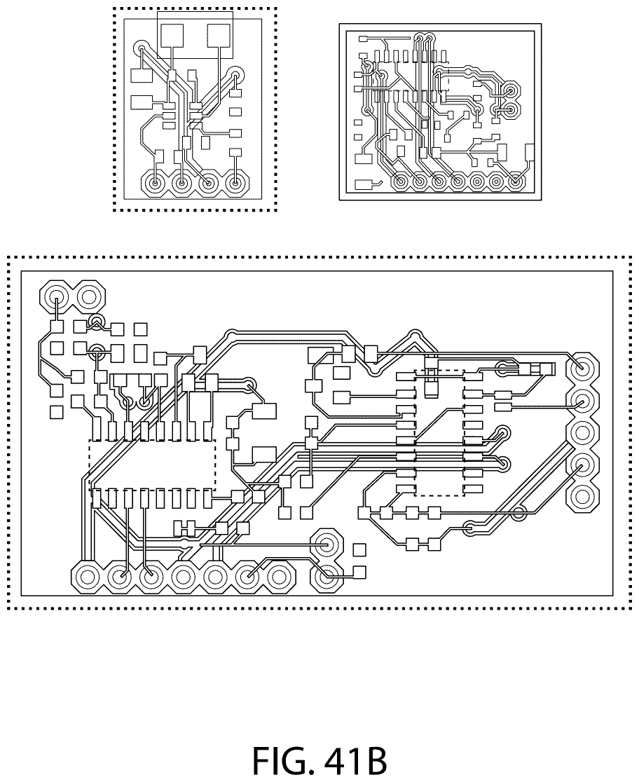

FIGS. 41A through 41B depict an electrical circuit of the sensor embedded in the collar device or the embodiments of the sensor incorporated within bedding for a pet. The MCU may comprise a Seeduino or Arduino nRF532840, preferably the latter, which may provide lower power consumption, more GPIO pins, more coding compatibility, and by BLE enabled. The battery may be a 6V battery, which may be a K-state battery. A buck converter may be provided to drop power output to about 3.3V. Modularity may be an feature; e.g., different batteries may be provided. Up to about 28V batteries may be included in embodiments herein. These improvements may produce improved battery life. The improved battery life may be up to about 460 continuous hours of run time. The embodiment illustrated is a fully merged board containing all subsystems: (e.g., weight, respiration rate, heart rate, and temperature).

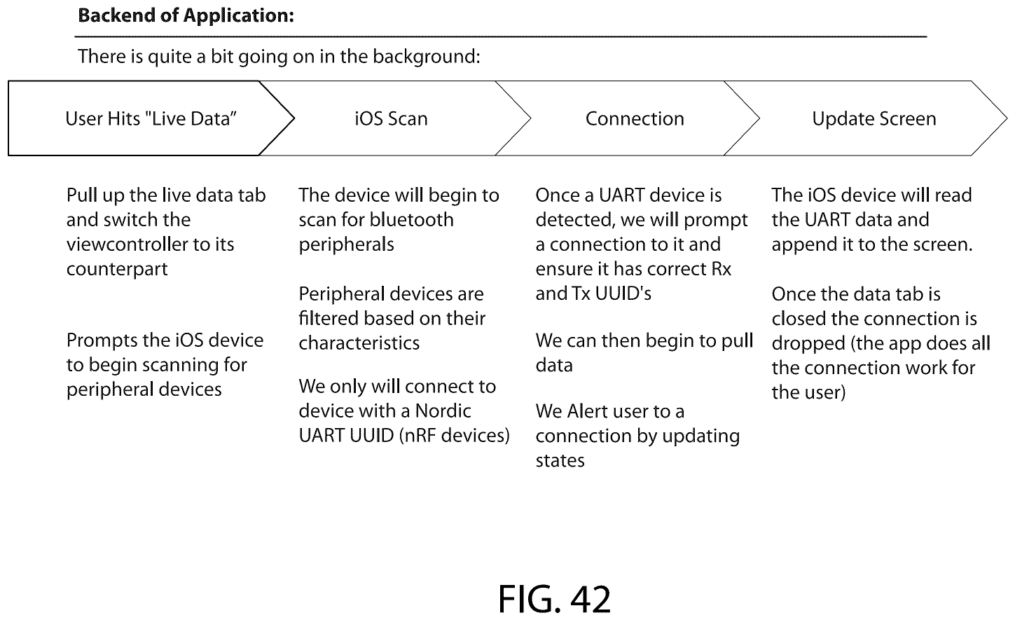

FIG. 42 depicts the metric detection system components in connection with a web-enabled device.

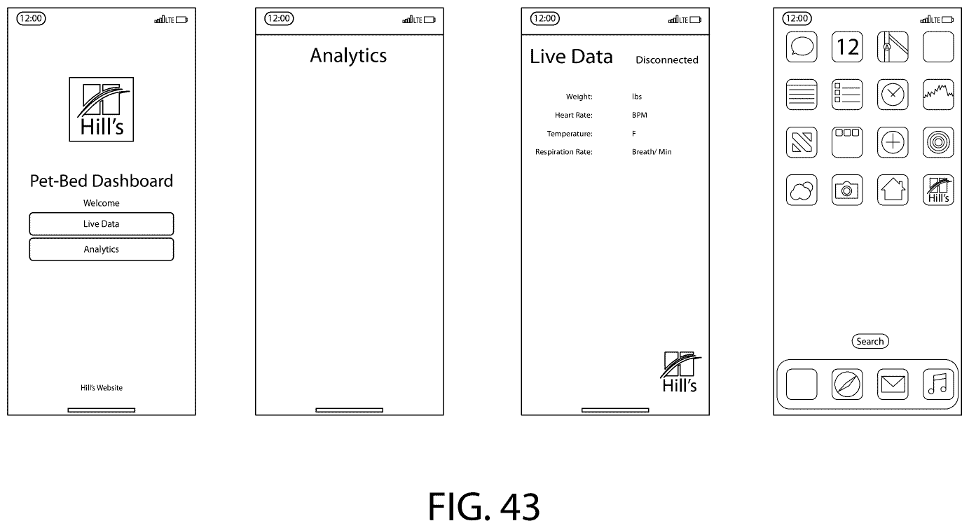

FIG. 43 depicts an image of a web-based application and computer program product displaying one or a plurality of metrics. The nRF may be connected to a user's computer. Or it may be powered by a batter. The Arduino may perform all of the BLE and send data packets. The result of the systems and methods herein could lead to estimates of aging that differ from chronological age. For example, calculations of the phenotypic age of a pet could lead to an age estimate that differs from chronological age of the pet, such as a medium-sized pet with a chronological age of 6 years (adult), but a phenotypic age more consistent with that of a pet several years older, for example 10 years (senior). Hence, in some embodiments, methods herein further comprise providing nutritional and care approaches typically used for senior or mature pets where phenotypic age indicates the pet is senior or mature despite a younger chronological age. The IN some embodiments, the methods comprise provision of appropriate care based on the phenotypic age, aging acceleration, aging deceleration, lifespan and/or healthspan for that pet. This could include modifications to schedules for veterinary examinations, diagnostic and screening tests. Such approaches could also include provision of appropriate nutrition based on the phenotypic age, aging acceleration, aging deceleration, lifespan and/or healthspan for that pet. This could include the provision of age- and/or lifestage-appropriate foods, such as Hill's Science Diet Adult 7+ Senior Vitality Chicken & Rice Recipe Dog Food, Hill's Science Diet Adult 7+ Senior Vitality Chicken & Rice Recipe Cat Food. This could also include provision of foods with appropriate nutrition profiles for the age- and/or lifestage of that pet. Examples of modifications to the nutrition profile of a food that could make the food more suitable for a senior or mature dog could include energy at a level of 3.0 to 4.0 kcal/dry matter (DM). Examples of modifications to the nutrition profile of a food that could make the food more suitable for a senior or mature cat could include energy at a level of 3.5 to 4.5 kcal/g dry matter (DM). For both dogs and cats, the appropriate nutrition for such an animal may need to be personalized based on the results of the phenotypic age, aging acceleration, lifespan or healthspan evaluation result.

These are examples for illustration only and in the case of an animal that is determined to have a phenotypic age younger than its chronological age, alternate nutritional approaches more suitable for a younger animal could be used. For example, calculations of the phenotypic age of a pet could lead to an age estimate that differs from chronological age of the pet in the opposite direction, such as a medium-sized pet with a chronological age of 10 years (senior), but a phenotypic age more consistent with that of a pet several years younger, for example 6 years (adult). The nutrition recommendations appropriate to an animal that is aging more slowly than would be expected would differ from those for older pets. For both dogs and cats, the appropriate nutrition for such an animal may need to be personalized based on the results of the phenotypic age, aging acceleration, lifespan or healthspan evaluation result.

In some embodiments, the disclosure provides a method for decelerating phenotypic aging of a companion animal in need thereof. The method comprises determining an index according to an aspect of the disclosure and providing personalized, customized, and/or tailored health, diet and nutrition measures for the companion animal according to the index for that companion animal. The method can also be considered to be a method of ameliorating accelerated aging or promoting/supporting decelerated aging, or maintaining an aging trajectory in the animal. In some embodiments, the personalized health measure comprises a diet that decelerates phenotypic aging and/or ameliorates accelerated phenotypic aging.

The following list of embodiments is exemplary of particular embodiments herein and is non-limiting to embodiments otherwise described herein.

1. A system for generating a multi-component aging index for an individual companion animal based on measuring at least one of digital biomarkers, traditional biomarkers and subjective assessment methods to predict phenotypic age and phenotypic age acceleration/deceleration in dogs and cats, the system optionally further comprising determining at least one of sex, neuter status, and lifestage.

2. The system according to embodiment 1, the system further comprising at least one of wearable devices to measure physical activity comprising walking, running, resting, jumping, sleep time, sleep quality and sleep regularity, subjective assessment via at least one of pet parent questionnaires and veterinary questionnaires, clinical characteristics comprising chronological age, weight, BCS, BFI, temperature, respiration rate, and heart rate, environmental sensors comprising sensors detecting at least one of location and location-based behaviors and activities comprising proximity to pet parent, play, timing and frequency of feeding, location of eating, drinking, urination and defecation, body posture, pose estimation, tail position, body position, movement tracking over time, repeated measures of clinical, digital and biological data, eating, drinking, urinating, defecating patterns, signs of emotional health, cognitive health, fear, anxiety, stress, dementia and social interaction with humans and other animals, and veterinary assessment of at least one of gastrointestinal disease, genitourinary disease, kidney disease, dermatological disease, respiratory disease, neurological disease, muscular disease, ophthalmological disease, auditory disease, cardiovascular disease, cancer, oral health, endocrine disease, infectious disease, immune function, inflammation, orthopedic disease, mobility, and pain.

3. The system according to embodiment 1 or 2, wherein the biomarker panels comprise two or more traditional biomarkers selected from CBC/chemistry parameters, fecal microbiome, fecal metabolites, urinary microbiome, urinary metabolites, blood metabolites, and blood biomarkers comprising albumin, creatinine, glucose, lymphocyte percent, mean cell volume, red cell distribution width, alkaline phosphatase, white blood cell count, SDMA, circulating peptides comprising Aβ42, circulating postbiotics, immunoglobulins, immunoglobulin M, growth hormone (GH)/insulin growth factor-1 (IGF-1), and DNA biomarkers, SNPs, and genetic variants.

4. The system according to embodiment 3, wherein the biomarker panels comprise three or more traditional biomarkers selected from CBC/chemistry parameters, fecal microbiome, fecal metabolites, urinary microbiome, urinary metabolites, blood metabolites, and blood biomarkers comprising albumin, creatinine, glucose, lymphocyte percent, mean cell volume, red cell distribution width, alkaline phosphatase, white blood cell count, SDMA, circulating peptides comprising Aβ42, circulating postbiotics, immunoglobulins, immunoglobulin M, growth hormone (GH)/insulin growth factor-1 (IGF-1), and DNA biomarkers, SNPs, and genetic variants.

5. The system according to embodiment 4, wherein the biomarker panels comprise four or more traditional biomarkers selected from CBC/chemistry parameters, fecal microbiome, fecal metabolites, urinary microbiome, urinary metabolites, blood metabolites, and blood biomarkers comprising albumin, creatinine, glucose, lymphocyte percent, mean cell volume, red cell distribution width, alkaline phosphatase, white blood cell count, SDMA, circulating peptides comprising Aβ42, circulating postbiotics, immunoglobulins, immunoglobulin M, growth hormone (GH)/insulin growth factor-1 (IGF-1), and DNA biomarkers, SNPs, and genetic variants

6. The system according to embodiment 5, wherein the biomarker panels comprise five or more traditional biomarkers selected from CBC/chemistry parameters, fecal microbiome, fecal metabolites, urinary microbiome, urinary metabolites, blood metabolites, and blood biomarkers comprising albumin, creatinine, glucose, lymphocyte percent, mean cell volume, red cell distribution width, alkaline phosphatase, white blood cell count, SDMA, circulating peptides comprising Aβ42, circulating postbiotics, immunoglobulins, immunoglobulin M, growth hormone (GH)/insulin growth factor-1 (IGF-1), and DNA biomarkers, SNPs, and genetic variants.

7. The system according to any of embodiments 1 to 6, wherein the multi-component aging index for an individual companion animal is further based on measuring epigenetic modifications including the DNA methylome.

8. The system according to any of embodiments 3 to 7, wherein the DNA biomarkers comprise one or a plurality of single nucleotide polymorphism (SNPs).

9. The system according to any of embodiments 2 to 8, wherein the wearable device is a Collar Mounted Activity Sensor (CMAS).

10. The system according to any of embodiments 2 to 9 further comprising one or more of a smart bed, a smart room and a smart toy and optionally one or more of a camera, computer vision, and audio units.

11. A method for decelerating phenotypic aging of a companion animal in need thereof comprising determining an index using the system of any one of embodiments 1to10 and based thereon providing customized health, dietary and/or nutrition measures to the companion animal.

12. The method according to embodiment 11, wherein the companion animal is an overweight animal.

13. The method according to embodiment 11, wherein the companion animal is an undernourished animal.

14. The method according to embodiment 11, wherein the companion animal is an animal that has difficulty maintaining a healthy weight.

15. The method according to embodiment 11, wherein the companion animal is an elderly animal or wherein the companion animal is an unknown age and determined to be experiencing advanced phenotypic age or aging acceleration via analysis of at least one traditional biomarker from the companion animal.

16. The method according to any of embodiments 11 to 15 wherein the personalized health measure comprises a diet that ameliorates accelerating phenotypic aging.

17. A system comprising: (a) a biosensor comprising: (i) a solid support comprising an internal cavity and external surface; (i) a band operably linked to an external surface of the solid support; (ii) an electrical circuit positioned within the internal cavity comprising at least a first position sensor and at least a first motion sensor; (b) at least one computer storage memory; and (c) a controller; wherein each of the sensors is in electrical communication with the controller.

18. A method of determining acceleration or deceleration of age of a subject comprising: (a) measuring one or a combination of activity metrics of the subject over a period of time; (b) determining a mobility score of the subject relative to a control subject of the same age; (c) classifying the subject as active if the mobility score is at or over the control mobility score for the age of the subject; or classifying the subject as inactive if the mobility score is under the control mobility score for the age of the subject.

19. The method of embodiment 18, wherein the subject is a companion animal.

20. The method of any of embodiments 18 or 19, wherein the activity metric is proximity to pet parent, play, timing and frequency of feeding, location of eating, drinking, urination and defecation, body posture (e.g., via pose estimation), tail position, body position, movement tracking over time, and incorporation of repeated measures of selected clinical, digital and biological data, eating, drinking, urinating, defecating and signs of emotional/cognitive health, such as fear, anxiety, stress, dementia and social interaction with humans and other animals tail movement, barking, jumping, scratching, speed of the animal in a direction, or sleeping.

21. The method of any of embodiments 18 to 20, wherein the period of time is no less than about one week.

22. The method of any of embodiments 18 to 21, wherein the period of time is no less than about 26 weeks.

23. The method of any of embodiments 18 to 22, wherein the step of measuring is performed by the system of embodiment 17.

24. The method of any of embodiments 18 to 23, wherein the step of measuring is performed by one or more weight sensors positioned on, under or proximate to the subject's bedding.

25. The method of embodiment 24, wherein the weight sensor(s) are a component of one or more biosensors positioned beneath the subject's bedding, and wherein the biosensor(s) comprise a top and bottom external surface and wherein the sensor(s) are capable of measuring the weight of the subject based upon the compression of the sensor(s) between the top and bottom external surfaces.

26. The method of embodiment 25, wherein the biosensor(s) comprises at least one additional sensor for measuring an activity metric other than sleeping.

27. The method of any of embodiments 18 to 26 further comprising a step of determining acceleration or deceleration of phenotypic age based upon the characterization of the subject as active or inactive.

27. A method of determining the phenotypic age of a subject comprising: (a) measuring one or a combination of activity metrics of the subject over a period of time; (b) determining a mobility score of the subject relative to a control subject of the same age; (c) classifying the subject as healthy if the mobility score is at or over the control mobility score for the age of the subject; or classifying the subject as unhealthy if the mobility score is under the control mobility score for the age of the subject; and/or (d) determining the age of the subject based upon the mobility score.

28. The method of embodiment 27, wherein the subject is a companion animal.

29. The method of any of embodiments 27 or 28, wherein the activity metric is at least one of proximity to pet parent, play, timing and frequency of feeding, location of eating, drinking, urination and defecation, body posture, pose estimation, tail position, body position, movement tracking over time, repeated measures of at least one of clinical data, digital data and biological data, or patterns of at least one of eating, drinking, urinating, defecating and signs of emotional health, cognitive health, fear, anxiety, stress, dementia, and social interaction with humans, interaction with other animals, tail movement, barking, jumping, scratching, speed of the animal in a direction, or sleeping.

30. The method of any of embodiments 27 to 29, wherein the period of time is no less than about one week.

31. The method of any of embodiments 27 to 30, wherein the period of time is no less than about 26 weeks.

32. The method of any of embodiments 27 to 31, wherein the step of measuring is performed by the system of embodiment 17.

33. The method of any of embodiments 27 to 32, wherein the step of measuring is performed by one or more weight sensors positioned on, under, or proximate to the subject's bedding.

34. The method of embodiment 33, wherein the weight sensor(s) are a component of one or more devices positioned beneath the subject's bedding, and wherein the device(s) comprise a top and bottom external surface and wherein the sensor(s) are capable of measuring the weight of the subject based upon the compression of the sensor(s) between the top and bottom external surfaces.

35. The method of embodiment 34, wherein the device comprises at least one additional sensor for measuring an activity metric other than sleeping.

36. A computer program product encoded on a computer-readable storage medium, wherein the computer program product comprises instructions for: (a) receiving data from one or a plurality of biosensors on a subject; (b) calculating a mobility score based upon the data; (c) determining the level of activity or age of a subject based upon the mobility score.

37. The computer program product of embodiment 36 further comprising a step of correlating the mobility score with the health of the subject.

38. The computer program product of embodiment 35, further comprising instructions for selecting a treatment for the subject based upon the phenotypic age or health of the subject.

39. A biosensor comprising: a top and a bottom exterior surface separated by a height, the exterior surface and the height defining an internal cavity comprising at least one of: (i) a gyroscope; (ii) at least a first pressure sensor; (iii) at least a first temperature sensor; (iv) at least a first accelerometer; and (v) a controller, wherein each of (i), (ii), (iii), and (iv) are in electrical communication with a controller by way of an electrical circuit.

40. The biosensor of embodiment 39, wherein the height is no more than about 3 inches.

41. The biosensor of embodiment 39 or 40, wherein the top and bottom exterior surfaces comprise a pliable materials chosen from: rubber, latex, vinyl or polyurethane, or a combination thereof.

42. The biosensor of any of embodiments 39 to 41, wherein the pressure sensor comprises at least one compression spring operably connected to the electrical circuit.

43. The biosensor of any of embodiments 39 to 42 further comprising a UV light spectrophotometer.

44. The biosensor of any of embodiments 39 to 43 further comprising a pH meter.

45. The biosensor of any of embodiments 39 to 44 further comprising a WiFi and Bluetooth communication antenna with a charging port.

46. The biosensor of any of embodiments 39 to 45 further comprising an amperometric hydrogen sulfide sensor.

47. A smart bed comprising a frame, a base, and sensory equipment.

48. A Smart room comprising at least one of (1) the biosensor of any of embodiments 39-46 and (2) the smart bed on embodiment 47.

49. A system comprising at least one of (1) the biosensor of any of embodiments 39 to 46, (2) the smart bed on embodiment 47, and (3) the smart room of embodiment 48, the system further comprising at least one computer storage memory; and a controller; wherein sensors in the biosensor, the smart bed, and/or the smart room are in electrical communication with the controller.

### EXAMPLES

Understanding a pet's health and wellbeing poses unique challenges, especially considering the communication gap between humans and animals. Exclusive reliance on owner reporting to understand pet health and wellbeing outside of the veterinary clinic presents several limitations due to the subjective nature of such reports and the potential for owners to overlook signs and behavioral changes that could be indicative of health issues.

The use of wearables for assessing animal behavior has emerged as a promising potential solution to these challenges (Neethirajan, Suresh. “Recent advances in wearable sensors for animal health management.” Sensing and Bio-Sensing Research 12 (2017): 15-29). Innovative tools such as wearable devices allow for continuous, objective monitoring of an animal's activities and behaviors (Griffies, Joel D., et al. “Wearable sensor shown to specifically quantify pruritic behaviors in dogs.” BMC veterinary research 14 (2018): 1-10.), thus enabling a more comprehensive and nuanced understanding of their health.

Establishing behavior norms is a crucial step in using wearable technology for monitoring and identifying early changes in health and wellbeing in animals. Norms provide a baseline against which deviations in behavior can be compared, enabling the early identification of potential health concerns. However, raw behavioral data alone might not be sufficient to derive meaningful insights, necessitating the development of sensitive and specific algorithms for behavior recognition and quantification.

In traditional behavioral research, one of the key methods used to analyze animal behavior is the identification and measurement of behavior bouts (Dawkins, Marian Stamp. Observing animal behavior: design and analysis of quantitative data. Oxford University Press, 2007.). These bouts represent discrete episodes of a specific behavior, allowing researchers to investigate the frequency, duration, and temporal distribution of an animal's activities. However, this method of analysis may not capture the full complexity of an animal's behavioral patterns, as it often treats each bout as an isolated event without considering the broader context of the animal's overall activity pattern.

A range of derived metrics can be used in the analysis of animal behavior. One such metric, sessionization, involves the categorization of prolonged periods of continuous behavior into ‘sessions’, treating these episodes not as isolated events, but rather as part of an ongoing behavioral sequence. It provides a comprehensive view of an animal's behavioral patterns by examining not only the individual behavior bouts but also the structure and continuity of these bouts within larger time intervals. Sessionization moves beyond the traditional measurement of isolated behavior bouts to consider the broader temporal landscape of an animal's behaviors. It acknowledges that a single bout of behavior does not occur in isolation but is instead part of a continuous stream of activity. By considering these broader patterns, it is possible to uncover additional layers of information, such as the regularity of certain behaviors, the sequencing of different behaviors, and the overall rhythm of an animal's activity cycle.

While in some cases, subjective assessments on their own may be accurate representations of an animal's behavior, in other cases, such assessments may leave room for improvement in terms of reliability and consistency. Understanding how well such subjective assessments agree with the objective measurements provided by sensor data will enable researchers to understand the relationship between observable dog behaviors and their perceived health status as well as providing them with the option to collect continuous, automated, objective data in situations where it may be difficult to obtain adequate information through subjective assessments. Furthermore, in some situations, combining subjective assessments—such as owner observations and veterinary evaluations—together with automated assessments generated through wearables, can provide a more holistic understanding of an animal's wellbeing. This interplay can aid in fine-tuning the analysis and interpretation of data generated by wearables.

The example presents a preliminary examination of a large, and growing, longitudinal dataset collected from companion dogs. This dataset leverages wearable technology, derived metrics, and subjective assessments to provide an extensive overview of animal health and wellbeing.

### Methodology

### Participants and Setting

Participants were recruited from the pool of current employees of Hill's Pet Nutrition. Employees who owned dogs were invited to enroll in a cohort study involving a wearable device and questionnaires, invitations were issued from 2020 through 2023. Participants were provided with a collar-worn wearable device containing a triaxial accelerometer at the time of study enrollment, along with instructions for setting up, charging and using the device. Complementing this objective data collection, subjective assessments of each dog's health and wellbeing were also collected. To this end, participants were intermittently invited to complete questionnaires through an email notification, all questionnaires were completed online.

### Questionnaire Data Collection

The questionnaires administered to study participants were carefully chosen to cover various aspects of a dog's health and quality of life. They included the Canine Brief Pain Inventory (CBPI) (Brown D C, Boston R C, Coyne J C, Farrar J T: Development and psychometric testing of an instrument designed to measure chronic pain in dogs with osteoarthritis. American Journal of Veterinary Research 68:631-637; 2007) to evaluate pain and its impact on the dog's life; the Sleep and Night Time Restlessness Evaluation Score (SNoRE) (Knazovicky, David, et al. “Initial evaluation of nighttime restlessness in a naturally occurring canine model of osteoarthritis pain.” Peer J. 3 (2015): e772.) questionnaire for sleep quality; an internally developed Aging questionnaire to monitor signs of aging; and a Quality of Life (QoL) (Lavan R P. Development and validation of a survey for quality of life assessment by owners of healthy dogs. Vet J. 2013 September; 197(3):578-82. doi: 10.1016/j.tvjl.2013.03.021. Epub 2013 Apr. 29. PMID: 23639368) questionnaire to assess overall wellbeing. In addition, the outcomes of veterinary assessments as reported by pet owners (“reported veterinarian assessments”) were collected using questionnaires administered to the pet owner.

The CBPI questionnaire was administered in 2023 and received 168 responses, the SNoRE questionnaire was administered in 2021 and received 287 responses, the Aging questionnaire was completed in 2022 and received 166 responses and the QoL questionnaire [1] was administered in 2022 and received 154 responses. Wearables data was not available for all participants dogs that completed the questionnaires.

### Wearable Data Collection

The collar-worn triaxial accelerometers worn by dogs participating in the study allowed for 24/7 recording of movement data. These accelerometers were set to capture data at a rate of 100 Hz with a sensitivity of ±8 g, ensuring the precision and accuracy of the movement data recorded.

The raw accelerometer data was transmitted wirelessly to the cloud and processed through a classification pipeline designed to apply a range of behavior recognition models (algorithms) to the accelerometer data. This labels or models the dogs' behaviors, grouping motion data into distinct categories including running, walking, scratching, shaking, resting, and sleeping. Algorithms have been shown to perform with high sensitivity, specificity and accuracy (Griffies, Joel D., et al. “Wearable sensor shown to specifically quantify pruritic behaviors in dogs.” BMC veterinary research 14 (2018): 1-10, which is incorporated herein by reference as if fully set forth), providing a solid foundation for the analysis of animal behavior in this context. Examples of classification pipelines, for various types of data, can be found in U.S. Pre-grant Publication Nos. 2022/0087229 (System and Method for Monitoring Motion of an Animal), 2022/0044788 (System and Method for Determining Caloric Requirements of an Animal), US 2022/0104464 (System and method for associating a signature of an animal movement and an animal activity), and 2022/0039358 (System and Method for Determining Caloric Requirements of an Animal Based on a Plurality of Durational Parameters). Data collected by any means herein or by any sensor herein may be analyzed as set forth in any one or more of the foregoing.

During the course of the study, data was collected continuously from the collar-worn triaxial accelerometers affixed to the collar of each client-owned dog. This methodology enabled the accumulation of an extensive dataset capturing the nuances of each dog's movement and behaviors in real-time over a period of over 3 years, with recruitment throughout the period.

Wearables data for each participant in the 7 days prior to the completion of each questionnaire was used for association analysis with questionnaire results. All available data was used when comparing session metrics between subgroups.

### Targeted Subgroup Analysis

This portion of the work was conducted using a diverse cohort of 188 client-owned dogs. Metric validation was focused on a distinct set of dogs from within this cohort. The set was designated as “Aging Evaluation” and comprised two subgroups: ‘Aging signs’ and ‘No reported aging signs’. The ‘Aging signs’ subgroup (n=53) included dogs that met our defined aging inclusion criteria, i.e., over the age of 5, over 15 lbs, not suffering from a number of preselected conditions, already eating a therapeutic diet, and were reported to have been diagnosed with an age-related condition. The ‘No reported aging signs’ subgroup (n=89) encompassed dogs that, while meeting the aging inclusion criteria, had not been reported to have been diagnosed with any age-related condition.

### Data Analysis

### Sessionization Metrics

Our study explores the concept of sessions, often referred to as bouts, in the context of animal behavior. Here, we specifically use the term to denote the extended display of a target behavior that remains robust against temporary deviations. We define a session through three parameters: minimum length, break threshold, and a minimum number of events. The emphasis of this study is primarily on the first two parameters, while the third parameter—the minimum number of events—was set to be a single event for the context of our research.

The minimum length parameter plays a crucial role in shaping the interpretation of the outcome. The minimum length pertains to the shortest duration for which the target behavior needs to be exhibited (including breaks) for the period to qualify as a session. This was chosen based on plausible durations for an exercise bout, with three primary durations taken into account—15 minutes, 5 minutes, and 1 minute.

The break threshold refers to the maximum permissible gap between instances of the target behavior before the ongoing session is considered to have ended. Finding the right balance in determining the break threshold is critical; too short a threshold could result in a surplus of prematurely discarded sessions, while too long a threshold might lead to the inappropriate merging of distinct sessions. The current study examined a range of values, ultimately establishing that a threshold of 10% is apt due to its position at the inflection point, following a slow initial increase and preceding an exponential rise.

A session is considered to be initiated with the first instance of the target behavior. Subsequent gaps in the target behavior are evaluated based on their length. If such a gap surpasses the break threshold, the ongoing session is deemed to have ended. A new session is then considered to have begun with the next occurrence of the target behavior. Lastly, all sessions that meet or exceed the minimum length are included in the final count. In the context of this study three target behaviors were examined: walking, running and either walking or running, called ‘active’.

### Validation of Sessionization Using Aging Evaluation Data

In this study, we postulated a hypothesis centered on dogs identified as having age-related health conditions. We hypothesized that these dogs would exhibit lower average daily session counts across different types of physical activities—namely walking, running, and being active in general—for three defined minimum lengths: 15 minutes, 5 minutes, and 1 minute. To test this hypothesis, we generated session metrics from two distinct groups within an aging canine population: a control group comprising dogs that had not been reported to have signs of an age-related condition, and a target group made up of dogs that had been identified as having signs of an age-related condition. The intent was to examine and compare the daily session counts between these two groups.

The non-parametric Mann-Whitney U test was applied to discern the difference between the control and target groups in terms of the number of sessions and total duration for walking, running, and general active behavior. The selection of the Mann-Whitney U test was motivated by its ability to compare non-normal distributions of independent variables between two groups.

### Subgroup Comparisons

Extensive metadata was collected for the cohort, enabling us to develop an overview of the various demographic groups within the population, see Table 1 (below). These groups were formulated based on several key characteristics, including age, living conditions (indoor, outdoor, or both), sex and reproductive status (neutered male, neutered female, intact male, intact female), breed type (purebred versus mixed breed). This metadata was pivotal in the study as it provided rich contextual information about the diverse characteristics of our cohort. Session variables were compared between these subgroups using the Mann Whitney U test. Visualizations of group means were created to allow for a visual comparison of the mean metric values between groups.

TABLE 1
Subgroup Participant Count

| Metric | Group | N |
|---|---|---|
| Age | Junior | 14 |
| | Adult | 53 |
| | Senior | 64 |
| Weight (lbs.) | <25 | 23 |
| | 25-55 | 34 |
| | >55 | 52 |
| Living Condition | Indoor | 63 |
| | Outdoor | 1 |
| | indoor_outdoor | 50 |
| Sex | Male | 52 |
| | Female | 62 |
| Neuter Status | Neutered | 49 |
| | Intact | 5 |
| Breed | Mixed | 44 |
| | Purebred | 70 |

### Owner Assessments

The relationship between the average weekly behavior rates and the responses collected from questionnaires was another critical aspect of the study. We sought to explore how the physical activity levels of dogs, as measured through our defined session metrics, correlated with the assessments provided by their owners.

To investigate this, Spearman's rank correlation coefficient, a non-parametric measure of the strength and direction of association between two ranked variables, was employed —. This statistical tool is particularly appropriate for this study, given its ability to identify both linear and monotonic relationships, providing a robust measure of correlation irrespective of the distribution of data.

Through the application of Spearman's rank correlation coefficient, we were able to evaluate the correlations between the activity metrics derived from our session-based approach and the subjective assessments offered by the owners. This allowed us to not only validate our activity metrics but also to understand the relationship between observable dog behaviors and their perceived health status.

We conducted Mann-Whitney U tests to determine differences in activity between the control group (dogs without reported signs of an age-related health condition) and the target group (dogs with reported signs of an age-related health condition). Specifically, we tested for differences in the counts of walking, running, and general active sessions, with a minimum duration of 1, 5, and 15 minutes (60, 300 and 900 seconds respectively). U statistics and p-values are listed in Table 2. Violin plots illustrate the distribution of values for each metric, including the long tails of increasingly higher values. Visually, the difference between the groups appears to be minor, but is apparent in the length of the tails, see FIG. 1, and these differences are borne out in the significance tests performed.

Table 2 Results of Mann-Whitney U tests between dogs with signs of aging and dogs with no signs of aging.

### Results

### Validation of Sessionization on Aging

We employed the Mann-Whitney U test to determine differences in activity between the control group (dogs without reported signs of an age-related health condition) and the target group (dogs with reported signs of an age-related health condition). Specifically, the teste was applied to the counts of walking, running, and general active sessions, with a minimum duration of 1, 5, and 15 minutes (60, 300, and 900 seconds, respectively). U statistics and p-values are listed in Table 2. Violin plots illustrate the distribution of values for each metric, including the long tails of increasingly higher values. The difference between the groups is minor, but apparent in the length of the tails, see FIGS. 29A and 29B.

TABLE 2
Output of Manny-Whitney U Test on Aging vs. Non-Aging

| Variable | U Statistic | Mean Target | Mean Control | P Value |
|---|---|---|---|---|
| walking_900 | 544 | 1.179 | 1.816 | 0.045 |
| running_900 | 479.5 | 0.059 | 0.296 | 0.007 |
| active_900 | 496 | 1.211 | 2.045 | 0.013 |
| walking_300 | 559 | 0.655 | 1.100 | 0.064 |
| running_300 | 503 | 0.151 | 0.526 | 0.014 |
| active_300 | 525 | 0.827 | 1.669 | 0.028 |
| walking_60 | 793.5 | 4.289 | 5.024 | 0.610 |
| running_60 | 586.5 | 0.454 | 1.737 | 0.105 |

### Subgroup Comparisons

In order to compare session variables across the groups, we implemented the Mann-Whitney U test. We applied this to each grouping pairwise to identify significant differences in the behavioral sessions of our defined subgroups, thereby shedding light on the variability of behaviors in different population subsets.

Our study incorporated a range of groups, each with its unique characteristics and profiles. Alongside the table, we have also included key figures that visually represent the most significant findings of our study.

TABLE 3
Most Significant Metrics from the Subgroup Comparison Analysis

| | | | | | Group | Group | p- |
|---|---|---|---|---|---|---|---|
| Behavior | Group 1 | | Group 2 | | 1 Mean | 2 Mean | value |
| running_60 | Male | | female | | 1.39 | 0.61 | 0.005 |
| walking_60 | 0-25 | Lbs. | 55+ | Lbs. | 1.98 | 4.23 | 0.008 |
| active_60 | 0-25 | Lbs. | 55+ | Lbs. | 2.40 | 5.31 | 0.008 |
| active_60 | Male | | female | | 7.31 | 4.03 | 0.040 |
| active_900 | neutered | | intact | | 1.51 | 2.90 | 0.052 |
| walking_60 | indoor | | indoor_out- | | 3.28 | 5.77 | 0.058 |
| | | | door | | | | |
| walking_300 | 25-55 | Lbs. | 55+ | Lbs. | 1.00 | 0.58 | 0.073 |
| running_900 | neutered | | intact | | 0.18 | 0.64 | 0.080 |
| running_300 | neutered | | intact | | 0.37 | 0.90 | 0.091 |
| active_60 | 0-25 | Lbs. | 25-55 | Lbs. | 2.40 | 6.85 | 0.095 |

We explored correlations between session metrics and responses from the Aging questionnaire, see Table 4. 15 minute running sessions are positively associated with the owners perception of the dog's stamina suggesting that more 15 minute running sessions are associated with increased stamina as reported by pet parents (R=0.43). More 15 min walking sessions were associated with reduced pet parent reported stamina (R=−0.18). Overall the number of 15 minute running sessions is not only the best differentiator between dogs with and without aging signs, it is also the best predictor of pet parent reported stamina. The second best differentiator between dogs with and without aging signs is 5 minute running sessions, and it is also the second best predictor of pet parent reported stamina, tied with 5 minute sessions of total activity. Higher scores in the aging questionnaire indicate fewer signs of aging.

TABLE 4
Spearman correlations for Aging questionnaire vs session metrics. Higher scores in the aging
questionnaire indicate fewer signs of aging. Increasing numbers mean increasing heat.

| | Correlation Matrix Heatmap | | | | | | | | |
|---|---|---|---|---|---|---|---|---|---|
| Aging_Stamina | −0.18 | 0.43 | 0.088 | 0.16 | 0.37 | 0.37 | −0.27 | 0.15 | −0.03 |
| Aging_Affection_{—} | −0.088 | 0.13 | 0.084 | 0.15 | 0.2 | 0.28 | 0.0024 | 0.11 | 0.15 |
| Frequency | | | | | | | | | |
| Aging_{—} | −0.14 | 0.17 | −0.021 | −0.094 | 0.033 | 0.0029 | −0.22 | 0.021 | −0.13 |
| Physically_Able | | | | | | | | | |
| Aging_{—} | −0.093 | 0.16 | 0.0032 | 0.0077 | 0.19 | 0.089 | 0.18 | 0.19 | 0.25 |
| Mentally_Able | | | | | | | | | |
| | walking_900 | running_900 | active_900 | walkin_300 | running_300 | active_300 | walking_60 | running_60 | active_60 |

Session counts were correlated with the SNoRE questionnaire responses see Table 5. Dogs that have more 15-minute walking sessions exhibit fewer movements during sleep (R=−0.35). The 15-minute running sessions correlate positively with reports of dreaming during sleep (R=0.44), suggesting that longer running activities might influence the depth or nature of a dog's sleep. Furthermore, these prolonged running sessions are notably associated with increased pacing during sleep, hinting at potential restlessness (R=0.55). When considering the overall sleep quality of the dog, it is evident that more frequent 1-minute total activity sessions correspond with owners perceiving a decline in their dogs' sleep quality (R=0.54). Overall, an increase in 15 minute sessions appears to be associated with better sleep quality, where more 1 minute sessions relate to poorer sleep quality. Higher scores in the SNoRE questionnaire indicate a negative impact on sleep quality.

TABLE 5
Spearman correlation matrix for snore vs session metrics. Higher scores in the SNoRE questionnaire
indicate a negative impact on sleep quality. Increasing numbers mean increasing heat.

| | Correlation Matrix Heatmap | | | | | | | |
|---|---|---|---|---|---|---|---|---|
| SNORE_MOVING | −0.35 | 0.23 | −0.36 | 0.071 | 0.4 | 0.078 | 0.054 | 0.054 |
| SNORE_TWITCHING | −0.47 | 0.13 | −0.47 | −0.12 | −0.073 | −0.059 | 0.14 | 0.14 |
| SNORE DREAMING | −0.15 | 0.44 | −0.14 | 0.25 | 0.2 | 0.36 | 0.24 | 0.24 |
| SNORE_SHIFTING | −0.21 | 0.32 | −0.22 | 0.029 | 0.37 | 0.029 | 0.16 | 0.16 |
| SNORE_VOCALIZING | −0.02 | −0.069 | −0.045 | −0.091 | −0.25 | −0.056 | 0.076 | 0.076 |
| SNORE_PACING | −0.052 | 0.55 | −0.052 | 0.34 | 0.47 | 0.34 | 0.052 | 0.052 |
| SNORE_THINK_{—} | −0.046 | 0.067 | −0.046 | −0.33 | −0.16 | −0.21 | 0.54 | 0.54 |
| SLEEP_QUALITY_7 | | | | | | | | |
| | walking_900 | running_900 | active_900 | walkin_300 | running_300 | active_300 | walking_60 | active_60 |

We correlated session metrics with Quality of Life (QoL) questionnaire responses in Table 6. Notably, dogs' responsiveness to their owners is strongly correlated with their daily average of 15-minute active sessions (R=0.83). Furthermore, a higher frequency of 15-minute running sessions is observed in dogs that owners believe sleep more (R=0.89). In contrast, dogs perceived to be in pain exhibit a strong negative correlation with 15-minute walking sessions (R=−0.93). Interestingly, dogs that frequently pant show a robust positive association with their average 15-minute active sessions (R=0.85), while dogs exhibiting a change in general health have a notable negative correlation with 15-minute running sessions (R=−0.89). Scores from the QoL questionnaire have been normalized so that higher scores indicate an improved quality of life.

TABLE 6
Spearman correlation matrix for QOL vs session metrics. Scores from the QoL questionnaire have been normalized
so that higher scores indicate an improved quality of life. Increasing numbers mean increasing heat.

| | Correlation Matrix Heatmap | | | | | | | | |
|---|---|---|---|---|---|---|---|---|---|
| Q0L_{—} | 0.22 | 0.66 | 0.83 | −0.25 | 0.49 | 0.43 | −0.21 | 0.49 | 0.62 |
| RESPONDS_{—} | | | | | | | | | |
| TO_OWNER | | | | | | | | | |
| QOL_{—} | 0.42 | 0.42 | 0.65 | 0.32 | 0.31 | 0.54 | −0.4 | 0.31 | 0.13 |
| ENJOYS_LIFE | | | | | | | | | |
| QOL_{—} | 0.42 | 0.42 | 0.65 | 0.32 | 0.31 | 0.54 | −0.4 | 0.31 | 0.13 |
| HAS_MORE- | | | | | | | | | |
| GOOD_DAYS_{—} | | | | | | | | | |
| THAN_{—} | | | | | | | | | |
| BAD_DAYS | | | | | | | | | |
| QOL_{—} | −0.098 | 0.89 | 0.8 | −0.56 | 0.66 | 0.44 | −0.53 | 0.66 | 0.59 |
| SLEEPS_MORE | | | | | | | | | |
| QOL_{—} | −0.93 | 0.52 | 0.29 | −0.71 | 0.69 | 0.4 | −0.5 | 0.69 | 0.68 |
| IN_PAIN | | | | | | | | | |
| QOL_{—} | −0.22 | 0.66 | 0.41 | −0.25 | 0.49 | 0.43 | −0.84 | 0.49 | 0 |
| MOVES_{—} | | | | | | | | | |
| NORMALLY | | | | | | | | | |
| QOL_NO_{—} | −0.13 | 0.066 | 0.22 | −0.22 | 0.018 | −0.079 | 0.36 | 0.018 | 0.46 |
| MOVEMENT | | | | | | | | | |
| QOL_{—} | 0.1 | 0.31 | 0.098 | 0 | −0.12 | −0.1 | −0.4 | −0.12 | −0.49 |
| AS_ACTIVE | | | | | | | | | |
| QOL_CLEAN | 0.42 | 0.42 | 0.65 | 0.32 | 0.31 | 0.54 | −0.4 | 0.31 | 0.13 |
| QOL_SMELLS_{—} | 0.42 | −0.7 | −0.65 | 0.32 | −0.46 | −0.4 | 0.53 | −0.46 | −0.39 |
| URINE_{—} | | | | | | | | | |
| SKIN_{—} | | | | | | | | | |
| IRRIRASTION | | | | | | | | | |
| QOL_DULL_{—} | −0.14 | 0.42 | 0.39 | −0.63 | 0.31 | 0 | 0.13 | 0.31 | 0.65 |
| DEPRESSED | | | | | | | | | |
| QOL_{—} | 0.29 | 0.65 | 0.85 | −0.12 | 0.48 | 0.49 | −0.27 | 0.48 | 0.54 |
| PANTS_{—} | | | | | | | | | |
| FREQUENTLY | | | | | | | | | |
| QOL_SHAKES | 0.066 | 0.56 | 0.93 | 0.11 | 0.66 | 0.83 | −0.38 | 0.66 | 0.74 |
| QOL_{—} | 0.49 | −0.89 | −0.49 | 0.78 | −0.66 | −0.32 | 0.78 | −0.66 | −0.34 |
| GENERAL_{—} | | | | | | | | | |
| HEALTH_{—} | | | | | | | | | |
| CHANGE | | | | | | | | | |
| QOL_QOL | 0.42 | 0.42 | 0.65 | 0.32 | 0.31 | 0.54 | −0.4 | 0.31 | 0.13 |
| | walking_900 | running_900 | active_900 | walkin_300 | running_300 | active_300 | walking_60 | running_60 | active_60 |

For most pain-related metrics, increased pain perceptions by owners align with reduced activity, see Table 7. High scores in the CBPI questionnaire indicate higher levels of pain, or impact of pain, with the exception of overall quality of life for which a higher score indicates a better quality of life. For instance, over a 7-day period, the least pain perceived has a negative association with many activity measures, including 15-minute active sessions (R=−0.23), signifying that higher baseline pain perception corresponds with decreased activity. The current pain perception further underscores this pattern, exhibiting a notably strong negative correlation with 60-second active sessions (R=−0.38). Contrastingly, a dog's ability to rise from a lying position reveals a slight positive link with 15-minute running sessions (R=0.13), indicating that this specific assessment of pain might not impede running bouts as much. Notably, a higher QoL, signifying better well-being, has a significant positive relationship with 60-second active sessions (R=0.38). This underscores that despite the challenges of pain, a dog's general well-being is associated with its activity levels.

TABLE 7
Spearman correlation matrix for CBPI vs session metrics. High scores in the CBPI questionnaire indicate higher levels of pain, or impact of pain,
with the exception of overall quality of life for which a higher score indicates a better quality of life. Increasing numbers mean increasing heat.

| | Correlation Matrix Heatmap | | | | | | | | |
|---|---|---|---|---|---|---|---|---|---|
| CBPI_{—} | −0.21 | −0.12 | −0.23 | −0.17 | −0.1 | −0.19 | −0.22 | −0.1 | −0.23 |
| LEAST_PAIN_7 | | | | | | | | | |
| CBPI_{—} | −0.15 | −0.22 | −0.2 | −0.12 | −0.19 | −0.16 | −0.24 | −0.043 | −0.27 |
| AVERAGE_{—} | | | | | | | | | |
| PAIN_7 | | | | | | | | | |
| CBPI_{—} | −0.21 | −0.2 | −0.25 | −0.19 | −0.17 | −0.22 | −0.35 | −0.17 | −0.38 |
| CURRENT_PAIN | | | | | | | | | |
| CBPI_ACTIVITY | −0.071 | −0.058 | −0.11 | −0.042 | −0.028 | −0.086 | −0.26 | −0.17 | −0.28 |
| CBPI_{—} | −0.086 | −0.03 | −0.14 | −0.064 | 0.037 | −0.12 | −0.26 | −0.1 | −0.29 |
| ENJOYMENT | | | | | | | | | |
| CBPI_RISE_{—} | −0.035 | 0.13 | 0 | −0.028 | −0.016 | −0.066 | −0.23 | −0.004 | −0.16 |
| FROM_LYING | | | | | | | | | |
| CBPI_WALK | −0.089 | −0.043 | −0.15 | −0.09 | −0.013 | −0.14 | −0.28 | −0.24 | −0.32 |
| CBPI_RUN | −0.15 | −0.22 | −0.19 | −0.12 | −0.19 | −0.16 | −0.25 | −0.047 | −0.28 |
| CBPI_CLIMB | −0.02 | −0.17 | −0.053 | −0.014 | −0.15 | −0.049 | −0.13 | 0.025 | −0.16 |
| CBPI_QOL_7 | 0.25 | 0.32 | 0.31 | 0.28 | 0.28 | 0.33 | 0.34 | 0.28 | 0.38 |
| | walking_900 | running_900 | active_900 | walkin_300 | running_300 | active_300 | walking_60 | running_60 | active_60 |

Our findings revealed some noteworthy trends in relation to the length of session metrics, see Table 2. For several of the longer session metrics—specifically those involving walking, running, and active behaviors over 15-minute and 5-minute intervals—dogs showing no signs of aging exhibited significantly higher values than those dogs exhibiting signs of aging. This trend was particularly pronounced in the case of the 15-minute sessions. These findings support the hypothesis that session metrics can differentiate between dogs that exhibit or do not exhibit signs of aging. Notably, the difference between the groups in these 15-minute sessions suggests that sessionization is a powerful tool in capturing the decline in stamina that typifies older dogs. This further emphasizes the utility of our methodological approach in elucidating nuanced changes in animal behavior that might otherwise be overlooked. Further examination of the data revealed an interesting trend at the 5-minute session level. The metrics for running and active behaviors were substantially higher in the dogs that do not exhibit signs of aging, see Table 2, suggesting that as dogs age, their ability to maintain longer periods of walking may be relatively preserved compared to their ability to sustain higher energy levels.

The Mann-Whitney U tests conducted across various dog subgroups did not reveal significant differences for all behavioral comparisons suggesting that not every comparison of ‘walking’, ‘running’, and ‘active’ states over various durations of time is associated with pet parent reported signs of aging, see Table 3. Nonetheless, certain intriguing patterns did emerge. In the ‘walking_60’ metric, a significant difference was detected between dogs weighing less than 25 lbs. and those over 55 lbs. (p=0.008), hinting that walking behavior patterns may vary based on weight. Furthermore, in the ‘running_60’ and ‘active_60’ categories, a significant difference was found between male and female dogs (p=0.005 and p=0.040, respectively), suggesting potential sex-based distinctions in running and overall activity levels. These findings suggest interesting directions for future research to explore the impact of weight and sex on dog behavior. Interestingly, we do not see significant differences between Adult and Senior dogs, a grouping that is based only on chronological age. However, when considering differences based on signs of aging, we see differences in behavior. This suggests that chronological age by itself may not be enough to distinguish pets with aging related behavior changes.

Our analysis of the correlations between session metrics and questionnaire responses indicates that while some correlations appear intuitive and compelling, others are less so, demonstrating that not all owner assessments are associated with behavioral data gathered through sessionization. Particularly noteworthy findings include correlations between activity measures and CBPI responses. These correlations were consistently in line with the notion that session metrics are impacted by the pain experienced by the dog, as assessed by the owner, and quality of life responses correlated positively with session metrics, suggesting that dogs perceived to have a higher quality of life exhibit more active behavior. This suggests that, despite their limitations, session metrics could still hold valuable insights into aspects of canine wellbeing that are closely linked to observable behaviors. The responses from the Quality of Life (QoL) questionnaire also showed some promising trends. Generally, there appears to be a positive correlation between QoL responses and session metrics, suggesting that dogs with higher QoL scores tended to exhibit more prolonged or more frequent behavioral sessions. Both 15 minute sessions of running and 15 minute sessions of total activity are the best predictors of dogs responding to owners, enjoying life and having more good days than bad days. 15 minute sessions for running and total activity are strongly negatively associated with the dog sleeping more or having a general change in health, but are positively associated with moving normally, panting frequently, keeping themselves clean, and shaking. Being able to walk, run, or be active for 15 minute sessions is strongly positively associated with quality of life. Taken generally this would suggest that the 15 minute session counts have potential to be a valuable component in assessing canine quality of life. In our analysis of the aging questionnaire responses, we found that overall the number of 15 minute running sessions is not only the best differentiator between dogs with and without aging signs, it is also the best predictor of pet parent reported stamina. The second best differentiator between dogs with and without aging signs is 5 minute running sessions, and it is also the second best predictor of pet parent reported stamina, tied with 5 minute sessions of total activity. This reinforces the suggestion that the long session durations may be able to capture an expenditure of energy that is beyond the capacity of dogs who are experiencing noticeable signs of aging. The SNoRE questionnaire, aimed at quantifying impact on sleep quality, on the whole suggests that an increase in 15 minute activity sessions are associated with better sleep quality, whereas more 1 minute sessions relate to poorer sleep quality. This could be because rather than engaging in a sustained period of activity, the dog engages in shorter periods of activity, interspersed with breaks, which might be expected if sleep quality is poor.

In light of these findings, we recognize the inherent complexities and challenges in using session metrics as a tool for interpreting animal wellbeing and health. It is important to consider also the directionality of the inferences being made. There is the possibility that, where the session metrics are aligned with owner assessments, it could either be because the metrics reflect the underlying condition of the dogs, or the ability of the dogs to sustain activity could be the factor that the owners perceive as being indicative of improvements in quality of life, sleep quality, pain experience or signs of aging.

The exploration of correlations between variables can be further extended. For instance, modeling interactions between variables may reveal complex relationships that simple correlations overlook. More in-depth studies could also examine relationships between owner and vet assessments, giving insights into disparities between professional and layman evaluations of canine health and wellbeing. Furthermore, the behavior exhibition rates in dogs with different conditions could be investigated, allowing for the impact of various ailments on canine behavior to be understood. The use of different questionnaires, potentially tapping into different aspects of dog behavior and wellbeing, can further enrich the dataset.

The integration of other measures of health and wellbeing can strengthen the understanding of canine pain and behavior. These measures could include physiological indicators such as heart rate, body temperature, or cortisol levels, which can serve as objective indices of stress or discomfort. Additionally, observational data on dogs' social behavior, sleep patterns, feeding and elimination habits, and other routine activities could serve as complementary data sources, providing a holistic picture of dogs' wellbeing.

The study revealed preliminary findings that provide confidence in the information that wearables can provide, with notable correlations between certain wearable device metrics and canine quality of life and pain measures, opening the door to further investigation and validation.

TABLE 8
Supplementary Table of Normalized Data

| Session | | | Group 1 | Group 2 | |
|---|---|---|---|---|---|
| Metric | Group 1 | Group 2 | Mean | Mean | p-value |
| running_900 | neutered | intact | 0.18 | 0.64 | 0.080 |
| active_900 | neutered | intact | 1.51 | 2.90 | 0.052 |
| running_300 | neutered | intact | 0.37 | 0.90 | 0.091 |
| running_60 | male | female | 1.39 | 0.61 | 0.005 |
| active_60 | male | female | 7.31 | 4.03 | 0.040 |
| walking_60 | indoor | indoor_outdoor | 3.28 | 5.77 | 0.058 |

| Behavior | Group1 | | Group2 | | Mean1 | Mean2 | p-value |
|---|---|---|---|---|---|---|---|
| walking_300 | 25-55 | Lbs. | 55+ | Lbs. | 1.00 | 0.58 | 0.073 |
| walking_60 | 0-25 | Lbs. | 55+ | Lbs. | 1.98 | 4.23 | 0.008 |
| active_60 | 0-25 | Lbs. | 25-55 | Lbs. | 2.40 | 6.85 | 0.095 |
| active_60 | 0-25 | Lbs. | 55+ | Lbs. | 2.40 | 5.31 | 0.008 |

An improved understanding of the aging process in a given pet would allow:
a. Pet parents and veterinarians to provide more personalized care based on the aging process in that pet. This could include modifications to schedules for veterinary examinations, diagnostic and screening tests, and vaccine schedules as well as age and/or lifestage-related accommodations or modifications (here and here), resulting in care that is better suited to the rate of aging and the lifestage of that pet.
b. Provision of appropriate nutrition based on the Phenotypic age, aging acceleration or aging deceleration results for that pet. This could include the provision of age- and/or lifestage-appropriate foods, such as Hill's Science Diet Adult 7+ Senior Vitality Chicken & Rice Recipe Dog Food, Hill's Science Diet Adult 7+ Senior Vitality Chicken & Rice Recipe Cat Food.
c. Provision of foods with appropriate nutrition profiles for the age- and/or lifestage of that pet.

As applied to dogs this could include feeding a dog with a food comprising at least one of or all of:
a. 3.0 to 4.0 kcal/g dry matter (DM)
b. fat levels between 7 and 15% DM
c. crude fiber of at least 2% DM
d. protein levels of 15 to 23% DM
e. phosphorous levels of 0.3 to 0.7% DM
f. sodium levels of 0.15 to 0.4% DM
g. at least 400 IU vitamin E/kg DM
h. at least 100 mg vitamin C/kg DM
i. 0.5 to 1.3 mg selenium/kg DM
j. calcium levels of 0.4% to 0.8% DM and
k. a calcium:phosphorous ratio of no less than 1.1.

As applied to cats, this could include feeding a cat a food comprising at least one of or all of:
a. 3.5 to 4.5 kcal/g dry matter (DM)
b. fat levels between 10 and 25% DM
c. crude fiber of up to 15% DM
d. protein levels of 30 to 45% DM
e. phosphorous levels of 0.5 to 0.7% DM
f. sodium levels of 0.2 to 0.4% DM
g. potassium levels of at least 0.6% DM
h. magnesium levels of 0.05% to 0.1% DM
i. lower urine acidifying potential, targeting urine pH of 6.4 and 6.6
j. at least 500 IU vitamin E/kg DM
k. 100 to 200 mg vitamin C/kg DM
l. 0.5 to 1.3 mg selenium/kg DM
m. calcium levels of 0.6 to 1.0% DM and
n. a calcium:phosphorous ratio of between 0.9:1 and 1.5:1.

The introduction or modification of cognitive and/or behavioral enrichment activities provided by the pet parent and/or veterinarian based on the Phenotypic age either in conjunction with provision of the above foods or on their own, including but not limited to modified exercise routines (e.g., more/less frequent or longer/shorter walks), play, interaction with smart toys or devices, introduction of novel toys or experiences, puzzles involving scent, puzzle feeders, and increased opportunities for interaction with people or other dogs.

Accordingly, some embodiments herein comprise feeding an animal a diet based on phenotypic age known or determined by any method or system herein. Some embodiments herein comprise modifying an animal exercise routine based on phenotypic age known or determined by any method or system herein. Some embodiments herein comprise modifying an animal play routine based on phenotypic age known or determined by any method or system herein. Some embodiments herein comprise modifying an animal smarty toy or device availability based on phenotypic age known or determined by any method or system herein. Some embodiments herein comprise introducing at least one of a new toy, experience, puzzle, puzzle involving scent, or puzzle feeders to an animal based on phenotypic age known or determined by any method or system herein. Some embodiments herein comprise increasing opportunities for interaction with one or more person or one or more other animal for an animal based on phenotypic age known or determined by any method or system herein. Some embodiments herein comprise decreasing opportunities for interaction with one or more person or one or more other animal for an animal based on phenotypic age known or determined by any method or system herein.

### Development of Biomarker-Driven Phenotypic Aging Models in Canines and Felines

Biological age (BA) is a measure of healthy aging that evaluates the accumulation of damage, physiological changes, and loss of function that occur in cells over time.[1] Circulating and non-circulating biomarkers can be evaluated within populations to estimate BA and predictors of BA can be leveraged further to assess mortality and morbidity risk.[2, 3]

While research involving humans is evolving and informed by multiple investigations, there is a paucity of analogous research in feline and canine models. Raj et al. developed an aging model using DNA methylation profiles in 130 felines but did not assess the association between modeled feline age and mortality. [4] Similarly, common “hallmarks of aging” such as immunosenescence are largely missing from the canine literature.[5]

We therefore collected biomarker data from large cohorts of canines and felines to develop models of phenotypic aging based on the methodology developed by Levine and colleagues[6] and subsequently validated in the US population by Liu et al.[7, 8]. The utility of biomarkers as predictors of both BA and mortality, as well as whether Phenotypic Age was under or overestimated by chronological age (Phenotypic Age acceleration), in canines and felines was also evaluated.

The following research objectives were examined and are presented in this example: 1. Developing phenotypic age for feline and canine, separately; and 2. Calculate Phenotypic Age Acceleration.

### Methods

### Feline Sample

A sample of 803 felines aged 0.1 to 19.5 years (mean±SD=5.7±4.5 years) at baseline were followed until death. Biomarkers were measured at baseline and each follow up visit. Our analytical sample limited the follow-up to 3 years and included 721 felines with complete biomarker data at baseline. Biomarkers measured at baseline were used to predict the risk of death over the 3 years of follow-up. Over this follow-up time, 109 (15.1%) felines died.

### Statistical Methods—Feline Analysis

Baseline characteristics were presented as means and standard deviations by death status. Two-sample t-tests were used to compare biomarkers between felines who died and those that did not die during the study period. Cox proportional hazard models were used to assess the univariate association between biomarkers and risk of death at any time during follow-up. Additionally, receiver operating characteristic curves (ROC) were used to evaluate all-cause mortality predictions and compared using the area under the curve (AUC).

Age and a total of 34 biomarkers were considered: platelets, red blood cells (RBC), hematocrit (HCT), hemoglobin, mean corpuscular volume (MCV), mean corpuscular hemoglobin (MCH), mean corpuscular hemoglobin concentration (MCHC), white blood cells (WBC), red cell distribution width (RDW), basophils, eosinophils, lymphocytes, monocytes, neutrophils, alanine aminotransferase (ALT), alkaline phosphatase (ALP), creatinine, blood urea nitrogen (BUN), blood urea nitrogen to creatine ratio, calcium, chloride, cholesterol, albumin, globulin, albumin to globulin ratio, glucose, phosphorus, magnesium, sodium, potassium, sodium to potassium ratio (Na/K ratio) bilirubin, total protein, and triglycerides.

Important biomarkers predictive of risk of death were selected using 3 methods:

First, stepwise backward selection method was used to select important biomarkers associated with the risk of death. This procedure resulted in the selection of 9 variables: HCT, MCH, WBC, RDW, Bun/Creat, BUN, Chloride, Sodium, and animal age (in years). (Model 1) The stepwise procedure was implemented in SAS.

Second, the Elastic Cox regression model was used to select clinical biomarkers associated with the risk of death. Ten-fold cross-validation was performed to select the parameter value, lambda, for the penalized regression, resulting in a lambda of 0.0812, a one standard deviation (SD) increase over the lambda with minimum mean-squared error during cross-validation. Of the 34 biomarkers and animal age included in the elastic Cox model, age and 7 biomarkers were selected: RBC, MCHC, MCH, MCV, RDW, Chloride, Triglycerides. (Model 2) The elastic net model was performed in R.

Third, we used variables retained by Liu et al. to develop a Phenotypic Age (PhenoAge) in humans.[7, 8] Nine out of 10 variables were available in our dataset and included: Creatinine, Glucose, MCV, Albumin, RDW, Lymphocytes, WBC, ALP, and animal age in years (Animal_age_yrs). (Model 3).

The selected variables were then included in a parametric proportional hazards model based on the Gompertz distribution.[6] The results were combined to estimate Phenotypic Age (in units of years) and presented below:

TABLE 9
Univariate associations between feline demographic
and biomarker characteristics and mortality

| | | |
|---|---|---|
| Variables | HR and 95% CI | p-value |

Sex

| | | | |
|---|---|---|---|
| Male | 1.78 (1.22, | 2.59) | 0.0027 |
| Female | 1.00 | | |
| Age, years | 1.35 (1.29, | 1.41) | <.0001 |
| PLT, k/μL | 1.00 (0.99, | 1.00) | 0.7159 |
| RBC, M/μL | 0.58 (0.54, | 0.62) | <.0001 |
| HCT, % | 0.87 (0.85, | 0.90) | <.0001 |
| HGB, g/dL | 0.76 (0.68, | 0.85) | <.0001 |
| MCHC, g/dL | 1.82 (1.65, | 2.01) | <.0001 |
| MCH, pg | 1.20 (1.17, | 1.23) | <.0001 |
| MCV, fl | 0.845 (0.81, | 0.89) | <.0001 |
| RDW, % | 1.05 (1.01, | 1.09) | 0.0065 |
| WBC, k/μl | 1.03 (1.00, | 1.07) | 0.0521 |
| Basophils, % | 0.12 (0.06, | 0.28) | <.0001 |
| Eosinophils, % | 0.84 (0.78, | 0.91) | <.0001 |
| Lymphocytes, % | 0.97 (0.95, | 0.99) | 0.0002 |
| Monocytes, % | 0.93 (0.84, | 1.02) | 0.1220 |
| Neutrophils, % | 1.04 (1.03, | 1.06) | <.0001 |
| Albumin, g/dL | 0.48 (0.29, | 0.77) | 0.0025 |
| Globulin, g/dL | 1.70 (1.39, | 2.09) | <.0001 |
| Albumin/Globulin ratio | 0.14 (0.06, | 0.31) | <.0001 |
| Total protein, g/dL | 1.60 (1.26, | 2.03) | 0.0001 |
| ALT, U/L | 1.00 (0.99, | 1.01) | 0.9045 |
| ALP, U/L | 1.00 (0.99, | 1.01) | 0.8166 |
| Bilirubin, mg/dL, per 0.1 | 0.43 (0.32, | 0.58) | <.0001 |
| mg/dL | | | |
| Creatinine, mg/dL | 1.40 (1.00, | 1.96) | 0.0466 |
| BUN, mg/dL | 1.04 (1.03, | 1.04) | <.0001 |
| Bun/Creatinine ratio | 1.03 (1.00, | 1.05) | 0.0156 |
| Sodium, mmol/L | 0.90 (0.87, | 0.94) | <.0001 |
| Potassium, mmol/L | 0.43 (0.29, | 0.64) | <.0001 |
| NA/K ratio | 1.01 (0.97, | 1.05) | 0.6019 |
| Chloride, mmol/L | 0.87 (0.83, | 0.90) | <.0001 |
| Calcium, mg/dL | 0.82 (0.65, | 1.03) | 0.0882 |
| Magnesium, mg/dL | 1.11 (0.98, | 1.27) | 0.1115 |
| Phosphorus, mg/dL | 0.71 (0.59, | 0.87) | 0.0006 |
| Glucose, mg/dL | 1.01 (1.00, | 1.01) | 0.0001 |
| Cholesterol, mg/dL | 1.00 (1.00, | 1.01) | 0.0513 |
| Triglycerides, mg/dL | 1.01 (1.00, | 1.01) | <.0001 |

### Phenotypic Age Derivation

This derivation is valid for the current dataset, assuming a single observation per animal. We have days=365.25*5 (5-year mortality risk for each animal). Full model, with all covariates:

$$mortality = {CDF}_{f} ( days , x ) = 1 - exp ( - exp ( {xb}_{f} ) ( exp ( {γ}_{f} days ) - 1 ) / {γ}_{f} ) \qquad ( 1 )$$

where b_{f}x=Σbᵢxᵢ+b₀=Σbᵢxᵢ+λ_{f}, the coefficients for the full model, where b₀=λ_{f}, the “rate” estimate, and γ_{f}, the scale estimate from Gompertz.

Univariate model, with the age covariate only:

$${CDF}_{a} ( days , age ) = 1 - exp ( - exp ( age {b}_{a} + {λ}_{a} ) ( exp ( {γ}_{a} days ) - 1 ) / {γ}_{a} ) \qquad ( 2 )$$

We have CDF_{f}(days,x)=CDFₐ(days,x). Set known constants κ_{f}=(exp(γ_{f}days)−1)/γ_{f} and κₐ=(exp(γₐdays)−1)/γₐ. This can be done because we assume a single observation per subject, that is, we have a scalar.

$$exp ( - exp ( \sum {b}_{i} {x}_{i} + {λ}_{f} ) {κ}_{f} ) = exp ( - exp ( age {b}_{a} + {λ}_{a} ) {κ}_{a} ) \qquad ( 3 )$$

$$exp ( \sum {b}_{i} {x}_{i} + {λ}_{f} ) {κ}_{f} = exp ( age {b}_{a} + {λ}_{a} ) {κ}_{a} \qquad ( 4 )$$

$$exp ( \sum {b}_{i} {x}_{i} + {λ}_{f} ) κ = exp ( age {b}_{a} + {λ}_{a} ) , \qquad ( 5 )$$

$$where κ = {κ}_{f} / {κ}_{a}$$

$$Result \text{:} age = \frac{- {λ}_{a} + \sum {b}_{i} {x}_{i} + {λ}_{f} + log κ}{{b}_{a}} = \frac{- {λ}_{a} + {xb}_{f} + log κ}{{b}_{a}} \qquad ( 6 )$$

$$where$$

$${xb}_{f} = \sum {b}_{i} {x}_{i} + {λ}_{f} , the full model ,$$

$$and$$

$$κ = {κ}_{f} / {κ}_{a}$$

$$for$$

$${κ}_{f} = ( exp / {γ}_{f} days ) - 1 ) / {γ}_{f}$$

$$and$$

$${κ}_{a} = ( exp ( {γ}_{a} days ) - 1 ) / {γ}_{a} .$$

For each Phenotypic Age measure, we calculated Phenotypic Age Acceleration (PhenoAgeAccel), defined as the residual resulting from a linear model when regressing Phenotypic Age on chronological age. Therefore, PhenoAgeAccel represents Phenotypic Age after accounting for chronological age (i.e., whether an animal appears older [positive value] or younger [negative value] than expected, physiologically, based on their age). [7, 8] The correlation between Phenotypic Age and chronological age, as well as the distribution of PhenoAgeAccel—the residual of Phenotypic Age regressed on chronological age—were evaluated.

Animals were then grouped into quintiles for PhenoAgeAccel, so that the highest quintile represented animals who were most at risk of death for their age—i.e., those whose Phenotypic Age was the highest relative to their chronological age. We then plotted Kaplan-Meier curves for animals in the highest 20% versus the lowest 20%. Next, receiver operating characteristic (ROC) curves were used to compare the 2-year mortality risk prediction of Phenotypic Age Measures and chronological age (by cohort; ages 0-5, 6-10, and 11-20 years) using the AUC.

### Canine Sample

A sample of 863 canines aged 0.1 to 16.5 years (mean±SD=5.2±5.1 years) at baseline were followed until death. Biomarkers were measured at baseline and each follow up visit. Our analytical sample limited the follow-up to 3 years and included 709 canines with complete biomarker data at baseline. Note: Canines with missing biomarker data were excluded from further analysis. Biomarkers measured at baseline were used to predict the risk of death over the 3 years of follow-up. Over this follow-up time, 89 (12.6%) canines died.

### Statistical Methods—Canine Analysis

The canine statistical analysis followed the same methodology as the feline statistical analysis. However, the following differences were noted: First, the model derived using a stepwise backward selection method resulted in the selection of 10 variables: MCHC, MCH, RDW, ALT, Albumin, Chloride, Sodium, Eosinophils, Lymphocytes, and animal age (in years). (Model 1). Second, the ten-fold cross-validation resulted in a lambda of 0.1225. Of the 34 biomarkers and animal age included in the elastic Cox model, age and 5 biomarkers were selected: RBC, MCH, RDW, ALT, and Na/K ratio. (Model 2). No changes were noted in the third model. (Model 3).

TABLE 10
Univariate associations between canine demographic
and biomarker characteristics and mortality

| Variable | HR and 95% CI | | P value |
|---|---|---|---|
| Sex | | | |
| Male | 0.78 | (0.51, 1.19) | 0.2540 |
| Female | 1.00 | | |
| Age, years | 1.55 | (1.45, 1.66) | <.0001 |
| PLT, k/μL | 1.01 | (1.00, 1.01) | <.0001 |
| RBC, M/μL | 0.48 | (0.43, 0.53) | <.0001 |
| HCT, % | 0.99 | (0.96, 1.01) | 0.2672 |
| HGB, g/dL | 0.96 | (0.89, 1.04) | 0.3329 |
| MCHC, g/dL | 1.12 | (0.93, 1.34) | 0.2413 |
| MCH, pg | 1.42 | (1.36, 1.48) | <.0001 |
| MCV, fl | 0.86 | (0.79, 0.94) | 0.0005 |
| RDW, % | 1.26 | (1.22, 1.29) | <.0001 |
| WBC, k/ul | 0.83 | (0.77, 0.91) | <.0001 |
| Basophils, % | 0.59 | (0.40, 0.86) | 0.0069 |
| Eosinophils, % | 0.99 | (0.91, 1.09) | 0.8935 |
| Lymphocytes, % | 1.02 | (1.00, 1.05) | 0.0724 |
| Monocytes, % | 1.01 | (0.96, 1.06) | 0.6368 |
| Neutrophils, % | 0.99 | (0.97, 1.01) | 0.3228 |
| Albumin, g/dL | 0.50 | (0.30, 0.82) | 0.0056 |
| Globulin, g/dL | 1.01 | (0.74, 1.37) | 0.9692 |
| Albumin/Globulin ratio | 0.72 | (0.41, 1.26) | 0.2484 |
| Total protein, g/dL | 0.86 | (0.67, 1.10) | 0.2211 |
| ALT, U/L | 1.003 | (1.002, 1.005) | <.0001 |
| ALP, U/L | 1.002 | (1.001, 1.002) | <.0001 |
| Bilirubin, mg/dL, per 0.1 | 0.55 | (0.41, 0.75) | 0.0001 |
| mg/dL | | | |
| Creatinine, mg/dL | 2.96 | (1.11, 7.87) | 0.0296 |
| BUN, mg/dL | 1.11 | (1.08, 1.14) | <.0001 |
| Bun/Creatinine ratio | 1.02 | (1.01, 1.04) | 0.0014 |
| Sodium, mmol/L | 0.99 | (0.97, 1.00) | 0.0409 |
| Potassium, mmol/L | 1.38 | (1.05, 1.83) | 0.0224 |
| NA/K ratio | 0.92 | (0.89, 0.94) | <.0001 |
| Chloride, mmol/L | 0.98 | (0.96. 0.99) | 0.0015 |
| Calcium, mg/dL | 0.63 | (0.49, 0.82) | 0.0006 |
| Magnesium, mg/dL | 3.22 | (1.66, 6.24) | 0.0005 |
| Phosphorus, mg/dL | 0.86 | (0.77, 0.97) | 0.0099 |
| Glucose, mg/dL | 0.97 | (0.95, 0.98) | <.0001 |
| Cholesterol, mg/dL | 1.01 | (1.00, 1.01) | 0.3647 |
| Triglycerides, mg/dL | 1.01 | (1.005, 1.01) | <.0001 |

### Results

The baseline characteristics of the feline and canine analytic cohorts are presented as the below Tables 11a and 11b, respectively. The mean age±SD of the feline cohort was 5.1±4.2 years and 63.8% were female. (Table 11a) The proportion of felines that died during the study period was significantly different by gender (p=0.0034) with a higher proportion of males dying vs females (20.3% vs 12.2%). Statistically significant differences in biomarkers between cats who were alive at the end of follow-up and those that died were observed for the majority of biomarkers (n=25/34 biomarkers) except platelet count, WBC, monocytes, ALT, ALP, creatinine, Na/K ratio, and cholesterol.

The mean age±SD of the canine cohort was 4.9±3.9 years and 54.7% were female. (Table 1b) In contrast with the feline cohort, the proportion of canines that died during the study period did not differ by gender (p=0.3281). Fewer biomarkers differed significantly between dogs who were alive at the end of follow-up and those that died (n=18/34 biomarkers).

TABLE 11a
Feline baseline characteristics according to survival status at the end of follow-up

| | All | | Alive | | Died | | |
|---|---|---|---|---|---|---|---|
| Variables | (n = 721) | | (n = 612) | | (n = 109) | | P-value |
| Sex | | | | | | | 0.0034 |
| Male | 261 | (36.2) | 208 | (79.7) | 53 | (20.3) | |
| Female | 460 | (63.8) | 404 | (87.8) | 56 | (12.2) | |
| Age, years | 5.1 | (4.24) | 4.2 | (3.24) | 10.4 | (5.24) | <.0001 |
| PLT, k/μL | 274.4 | (253.31) | 273.0 | (262.71) | 282.5 | (192.93) | 0.6580 |
| RBC, M/μL | 7.3 | (2.50) | 7.9 | (1.37) | 3.8 | (4.02) | <.0001 |
| HCT, % | 35.9 | (6.20) | 36.7 | (5.82) | 31.7 | (6.58) | <.0001 |
| HGB, g/dL | 11.3 | (1.87) | 11.4 | (1.78) | 10.5 | (2.10) | <.0001 |
| MCHC, g/dL | 31.5 | (1.66) | 31.2 | (1.43) | 33.2 | (1.88) | <.0001 |
| MCH, pg | 17.4 | (6.95) | 15.4 | (4.12) | 28.5 | (8.83) | <.0001 |
| MCV, fl | 45.9 | (4.21) | 46.3 | (3.77) | 43.6 | (5.63) | <.0001 |
| RDW, % | 26.4 | (4.74) | 26.2 | (4.52) | 27.4 | (5.73) | 0.0322 |
| WBC, k/μL | 11.0 | (5.18) | 10.8 | (5.08) | 11.8 | (5.68) | 0.0813 |
| Basophils, % | 0.4 | (0.44) | 0.4 | (0.45) | 0.2 | (0.32) | <.0001 |
| Eosinophils, % | 6.0 | (3.14) | 6.2 | (3.16) | 4.9 | (2.74) | <.0001 |
| Lymphocytes, % | 28.1 | (13.34) | 28.9 | (13.04) | 23.6 | (14.19) | 0.0001 |
| Monocytes, % | 4.2 | (2.06) | 4.2 | (2.01) | 3.9 | (2.34) | 0.1799 |
| Neutrophils, % | 61.4 | (13.38) | 60.2 | (13.06) | 67.8 | (13.35) | <.0001 |
| Albumin, g/dL | 3.3 | (0.38) | 3.3 | (0.37) | 3.1 | (0.40) | 0.0018 |
| Globulin, g/dL | 3.6 | (0.78) | 3.5 | (0.73) | 3.9 | (0.97) | <.0001 |
| Albumin/Globulin | 1.0 | (0.26) | 1.0 | (0.25) | 0.9 | (0.27) | <.0001 |
| ratio | | | | | | | |
| Total protein, g/dL | 6.8 | (0.78) | 6.7 | (0.76) | 7.0 | (0.84) | <.0001 |
| ALT, U/L | 57.9 | (38.50) | 57.7 | (34.89) | 58.5 | (54.69) | 0.8880 |
| ALP, U/L | 36.1 | (23.76) | 36.2 | (24.17) | 35.2 | (21.41) | 0.6837 |
| Bilirubin, mg/dL | 0.2 | (1.01) | 0.2 | (1.09) | 0.1 | (0.17) | 0.0348 |
| Creatinine, mg/dL | 1.4 | (0.50) | 1.4 | (0.41) | 1.4 | (0.83) | 0.2650 |
| BUN, mg/dL | 23.3 | (7.64) | 22.5 | (4.62) | 27.6 | (15.69) | 0.0010 |
| Bun/Creatinine ratio | 18.6 | (7.73) | 18.3 | (7.94) | 20.1 | (6.30) | 0.0121 |
| Sodium, mmol/L | 154.1 | (4.96) | 154.4 | (5.05) | 152.1 | (3.92) | <.0001 |
| Potassium, mmol/L | 4.3 | (0.52) | 4.3 | (0.52) | 4.0 | (0.46) | <.0001 |
| NA/K ratio | 37.8 | (4.69) | 37.7 | (4.53) | 38.1 | (5.53) | 0.5230 |
| Chloride, mmol/L | 120.2 | (4.27) | 120.7 | (4.28) | 117.6 | (3.07) | <.0001 |
| Calcium, mg/dL | 9.8 | (0.83) | 9.8 | (0.85) | 9.6 | (0.68) | 0.0290 |
| Magnesium, mg/dL | 1.9 | (0.66) | 1.9 | (0.70) | 2.1 | (0.28) | 0.0027 |
| Phosphorus, mg/dL | 5.0 | (1.29) | 5.1 | (1.26) | 4.5 | (1.33) | 0.0001 |
| Glucose, mg/dL | 99.0 | (40.69) | 96.8 | (36.29) | 111.3 | (58.35) | 0.0132 |
| Cholesterol, mg/dL | 157.3 | (53.17) | 155.7 | (51.44) | 166.2 | (61.52) | 0.0970 |
| Triglycerides, mg/dL | 52.7 | (62.80) | 42.6 | (34.68) | 109.5 | (125.16) | <.0001 |

TABLE 11b
Canine baseline characteristics according to survival status at the end of follow-up

| | All | | Alive | | Died | | |
|---|---|---|---|---|---|---|---|
| | (n = 709) | | (n = 620) | | (n = 89) | | |
| | Mean (SD) | | Mean (SD) | | Mean (SD) | | |
| Variables | or n (%) | | or n (%) | | or n (%) | | |
| Sex | | | | | | | 0.3281 |
| Male | 321 | (45.3) | 285 | (88.8) | 36 | (11.2) | |
| Female | 388 | (54.7) | 335 | (86.3) | 53 | (13.7) | |
| Age, years | 4.9 | (3.94) | 4.2 | (3.23) | 10.1 | (4.50) | <.0001 |
| PLT, k/μL | 306.2 | (114.71) | 295.7 | (101.39) | 379.3 | (165.48) | <.0001 |
| RBC, M/μL | 5.6 | (1.70) | 6.0 | (1.06) | 3.4 | (3.07) | <.0001 |
| HCT, % | 41.1 | (7.39) | 41.2 | (7.50) | 40.3 | (6.52) | 0.2966 |
| HGB, g/dL | 14.1 | (4.76) | 14.2 | (5.01) | 13.7 | (2.36) | 0.1672 |
| MCHC, g/dL | 33.9 | (1.18) | 33.9 | (1.13) | 34.0 | (1.50) | 0.4738 |
| MCH, pg | 24.3 | (3.26) | 23.6 | (1.60) | 29.8 | (5.72) | <.0001 |
| MCV, fl | 69.0 | (2.50) | 69.1 | (2.38) | 68.2 | (3.07) | 0.0079 |
| RDW, % | 17.6 | (4.53) | 16.6 | (2.32) | 24.9 | (8.09) | <.0001 |
| WBC, k/ul | 8.9 | (3.58) | 9.2 | (3.55) | 7.3 | (3.36) | <.0001 |
| Basophils, % | 0.9 | (4.61) | 1.0 | (4.92) | 0.4 | (0.78) | 0.0078 |
| Eosinophils, % | 2.2 | (2.32) | 2.2 | (2.38) | 2.1 | (1.85) | 0.9826 |
| Lymphocytes, % | 18.1 | (7.55) | 17.9 | (7.20) | 19.7 | (9.58) | 0.0945 |
| Monocytes, % | 6.6 | (4.23) | 6.6 | (4.28) | 6.9 | (3.83) | 0.5409 |
| Neutrophils, % | 72.1 | (10.40) | 72.3 | (10.23) | 70.8 | (11.53) | 0.2091 |
| Albumin, g/dL | 3.3 | (0.41) | 3.3 | (0.39) | 3.2 | (0.46) | 0.0313 |
| Globulin, g/dL | 2.5 | (0.69) | 2.5 | (0.68) | 2.5 | (0.71) | 0.8381 |
| Albumin/Globulin | 1.4 | (0.42) | 1.4 | (0.43) | 1.4 | (0.40) | 0.3735 |
| ratio | | | | | | | |
| Total protein, g/dL | 5.8 | (0.86) | 5.9 | (0.86) | 5.7 | (0.88) | 0.2067 |
| ALT, U/L | 43.9 | (47.07) | 40.2 | (26.66) | 69.7 | (109.79) | 0.0133 |
| ALP, U/L | 120.6 | (120.84) | 114.5 | (88.80) | 163.4 | (244.75) | 0.0651 |
| Bilirubin, mg/dL | 0.2 | (0.14) | 0.2 | (0.13) | 0.1 | (0.14) | 0.0002 |
| Creatinine, mg/dL | 0.7 | (0.22) | 0.7 | (0.21) | 0.7 | (0.28) | 0.1995 |
| BUN, mg/dL | 12.1 | (4.90) | 11.6 | (4.15) | 15.5 | (7.63) | <.0001 |
| Bun/Creatinine ratio | 20.1 | (10.95) | 19.5 | (10.89) | 23.8 | (10.67) | 0.0006 |
| Sodium, mmol/L | 148.0 | (7.58) | 148.2 | (5.27) | 146.5 | (16.27) | 0.3258 |
| Potassium, mmol/L | 4.4 | (0.70) | 4.4 | (0.68) | 4.5 | (0.86) | 0.1078 |
| NA/K ratio | 36.1 | (5.68) | 36.6 | (5.44) | 32.8 | (6.22) | <.0001 |
| Chloride, mmol/L | 112.1 | (6.14) | 112.4 | (4.58) | 109.9 | (12.26) | 0.0610 |
| Calcium, mg/dL | 10.5 | (0.88) | 10.6 | (0.88) | 10.2 | (0.82) | 0.0002 |
| Magnesium, mg/dL | 1.7 | (0.25) | 1.7 | (0.24) | 1.8 | (0.29) | 0.0039 |
| Phosphorus, mg/dL | 4.8 | (2.35) | 4.9 | (2.44) | 4.2 | (1.41) | 0.0002 |
| Glucose, mg/dL | 99.0 | (14.81) | 99.8 | (14.60) | 93.0 | (15.02) | <.0001 |
| Cholesterol, mg/dL | 208.7 | (63.34) | 208.0 | (61.78) | 213.5 | (73.48) | 0.5022 |
| Triglycerides, mg/dL | 59.8 | (36.53) | 57.7 | (35.83) | 74.7 | (38.04) | <.0001 |

### Univariate Models

The association between individual variables and mortality for felines and canines are presented in Tables 9 and 10, respectively. Among felines, each year of age was associated with a 35% unadjusted increase in the risk of mortality. Among single biomarkers, MCHC was associated with nearly double the risk of mortality (HR: 1.82; 95% CI: 1.65, 2.01). In canines, each year of age was associated with a 55% unadjusted increase in the risk of mortality. Magnesium (HR: 3.22; 95% CI: 1.66, 6.24) and creatinine (HR: 2.96; 95% CI: 1.11, 7.87) were the biomarkers most strongly associated with mortality on an individual basis.

### Feline Analysis

### Survival Analysis

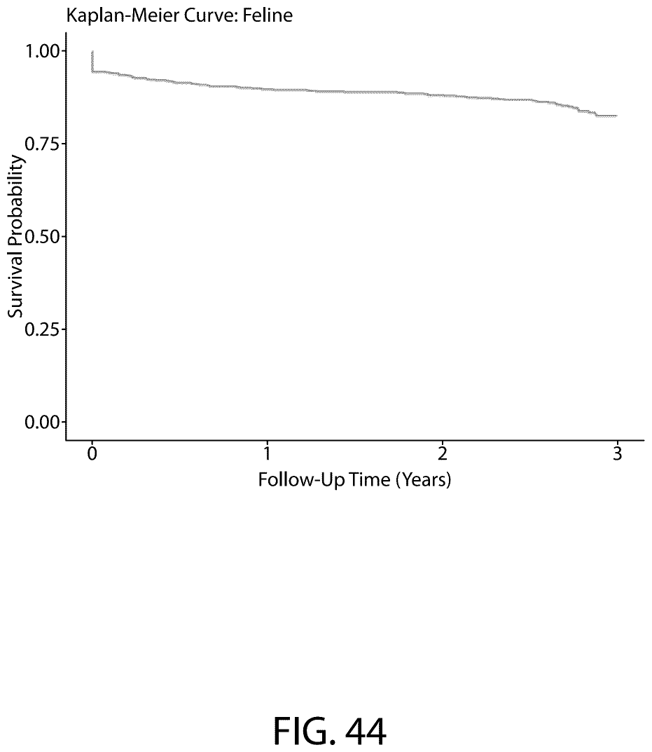

FIG. 44 illustrates overall survival among felines. As noted in Table 11a, 109 of 721 (15.1%) of felines died during the 3-year follow-up.

### Associations of Phenotypic Age and Mortality

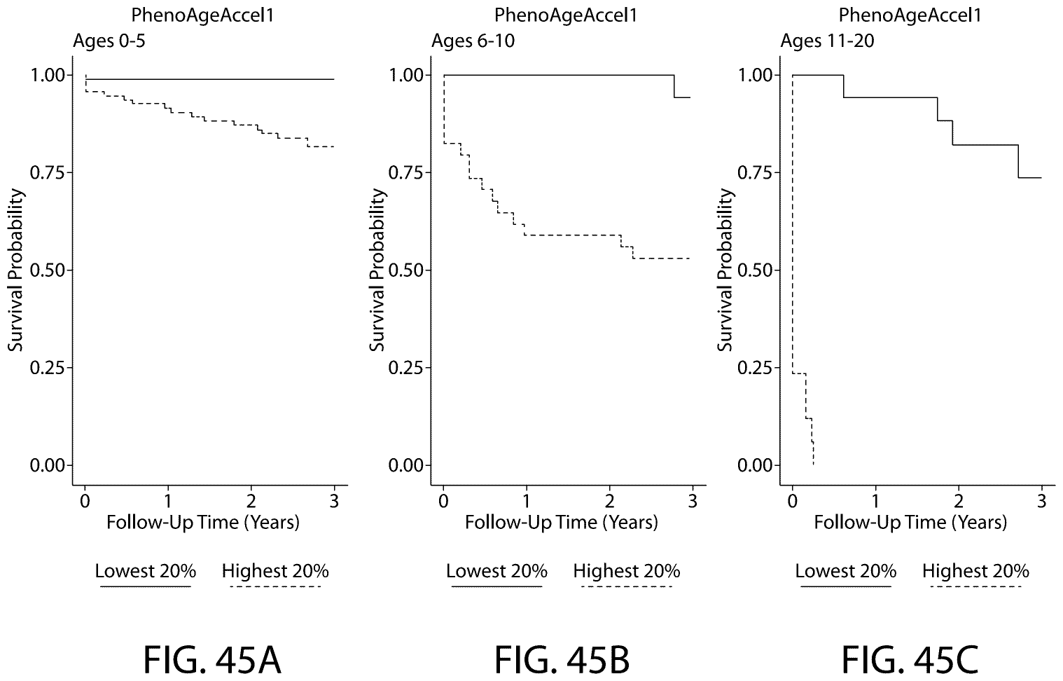

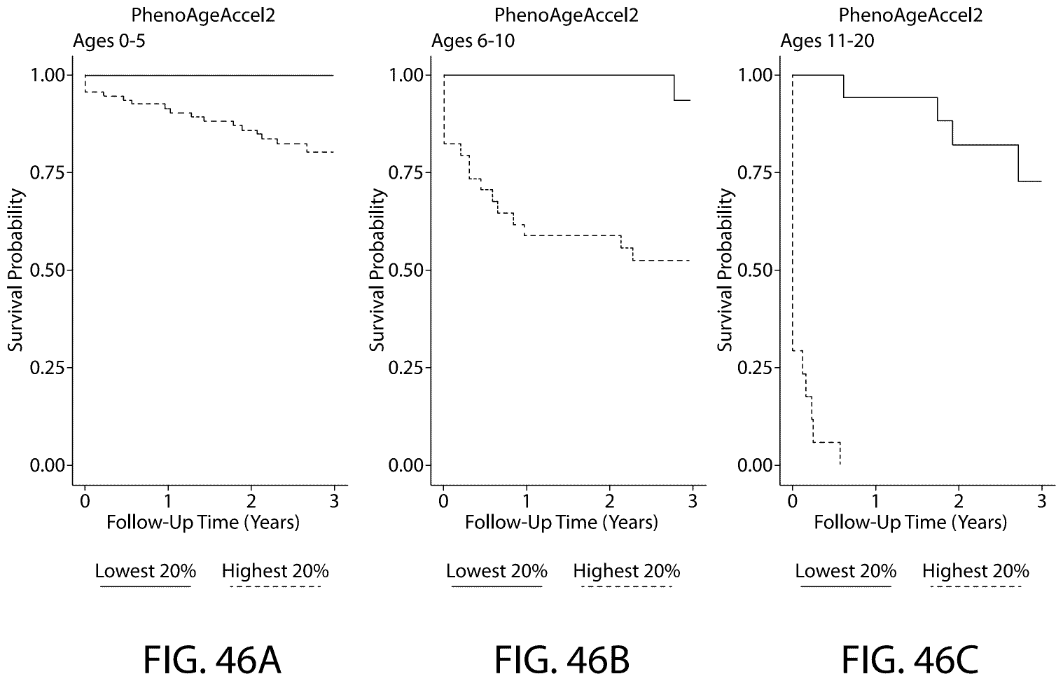

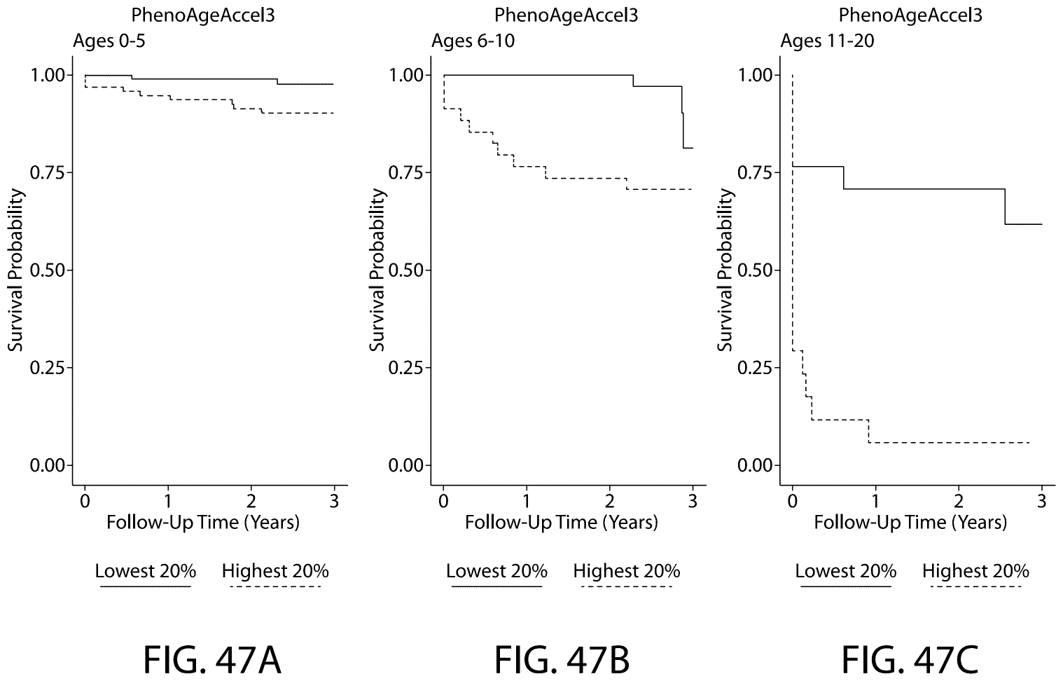

As shown in FIGS. 45A-45C, 46A-46C, and 47A-47C, felines with the highest Phenotypic Age relative to their chronological ages had steeper declines in survival over 3 years of follow-up, and differences grew between age intervals from felines ages 0-5 to felines ages 11-20 having the steepest decline in survival in the high risk group (highest 20% of PhenoAgeAccel). The difference in mortality between high and low PhenoAgeAccel was slightly attenuated in Model 3, which relied upon human PhenoAge predictors. For each of FIGS. 45A-47C, the y-axis indicates the survival rate, and the x-axis indicates follow-up time (in years). Similar trends by age cohort were observed across all models.

### Phenotypic Age Acceleration

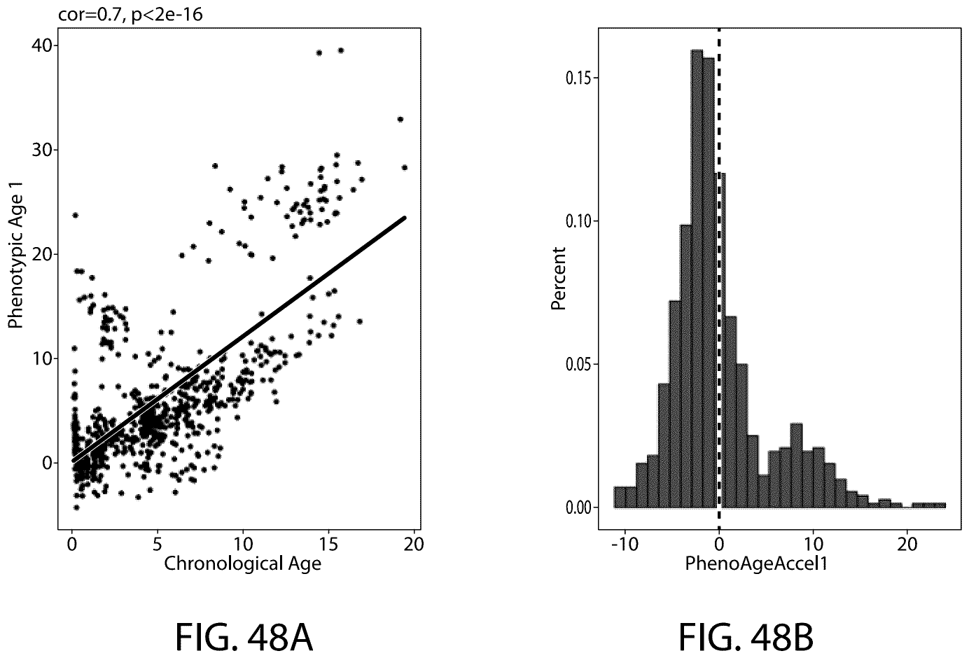

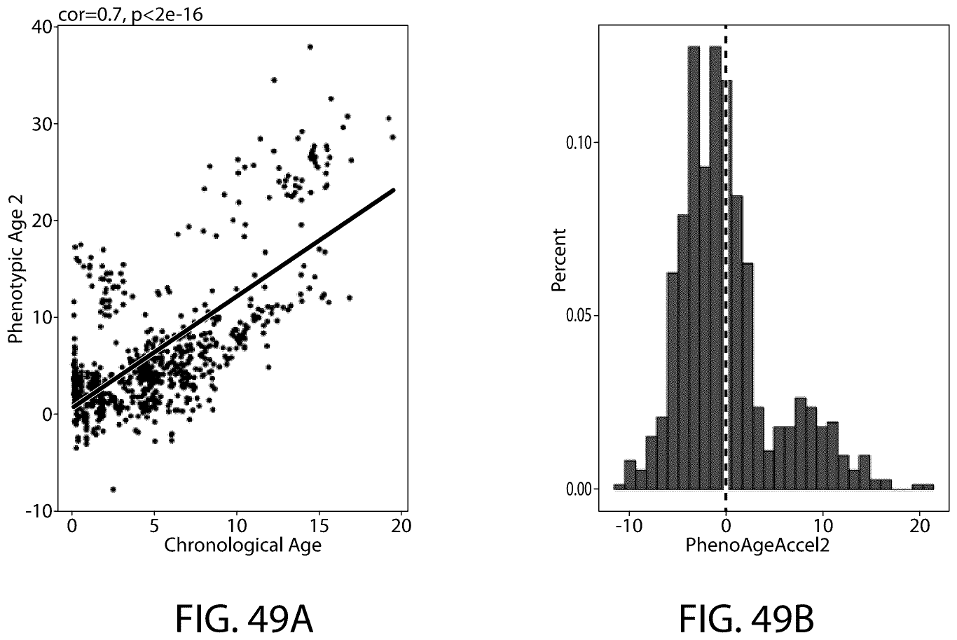

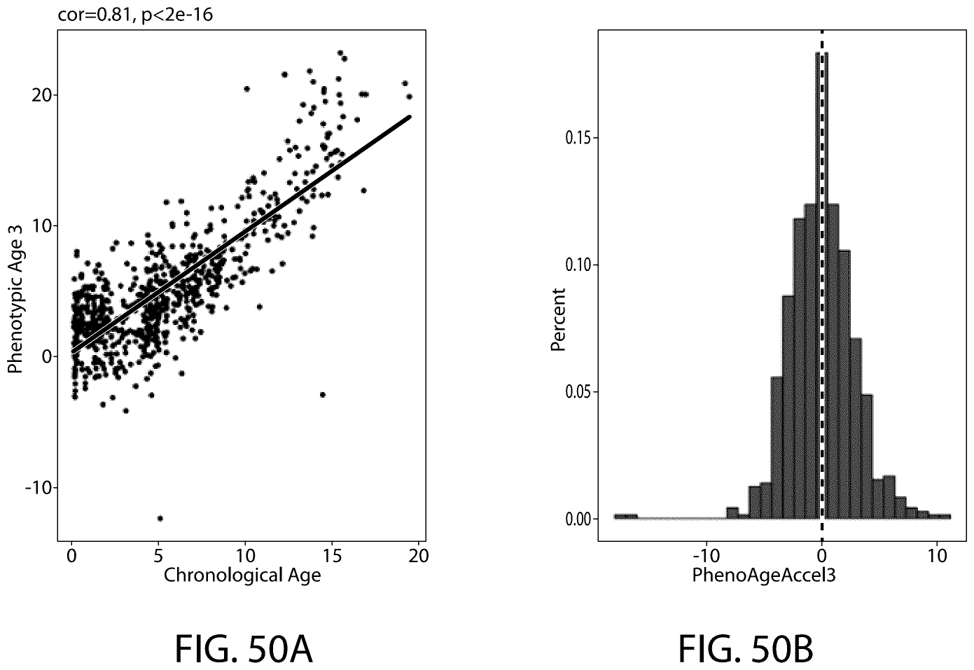

FIGS. 48A, 48B, 49A, 49B, 50A, and 50B illustrate the correlation between Phenotypic Age and chronological age, as well as the distribution of PhenoAgeAccel—the residual of Phenotypic Age regressed on chronological age. Phenotypic Age and chronological age are moderately-strongly correlated across models; part of this is due to the fact that age is in the Phenotypic Age measure. Phenotypic Age was moderately (r=0.7) correlated with chronological age in Models 1 and 2, which is partially due to the fact that the models include chronological age. The red line depicts the expected Phenotypic Age for each chronological age, with points above the line depicting people who were phenotypically older than expected, and points below the line depicting those who were phenotypically younger than expected. PhenoAgeAccel was fairly normally distributed but had a wider range than observed in humans in Liu et al. in all models. Phenotypic Age was more strongly (r=0.81) correlated with chronological age in Model 3 relative to Models 1 and 2.

### 2-Year Mortality

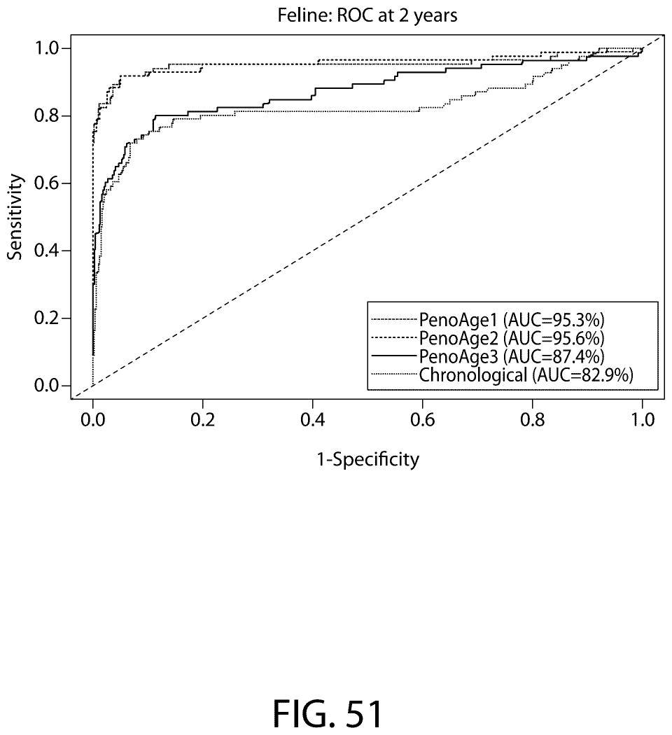

ROC curves (FIG. 51) in felines revealed that Phenotypic Age, with an area under the curve (AUC) of 0.953 in Model 1, 0.956 in Model 2 outperformed chronological age (0.829). Model 3 (0.874) did not predict 2-year mortality better than Models 1 and 2 relative to chronological age.

### Canine Analysis

### Survival Analysis

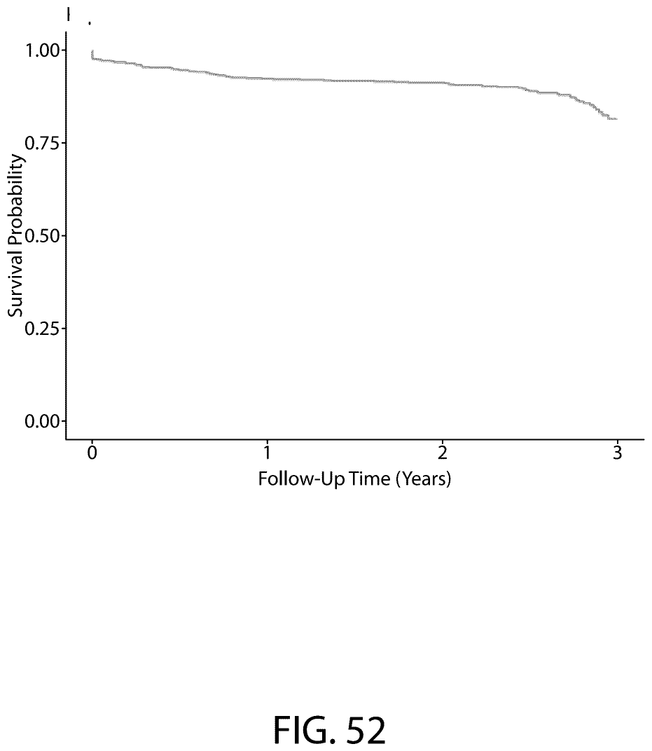

FIG. 52 illustrates overall survival among canines. As noted in Table 11b, 89 of 709 (12.6%) of canines died during the 3-year follow-up.

### Associations of Phenotypic Age and Mortality

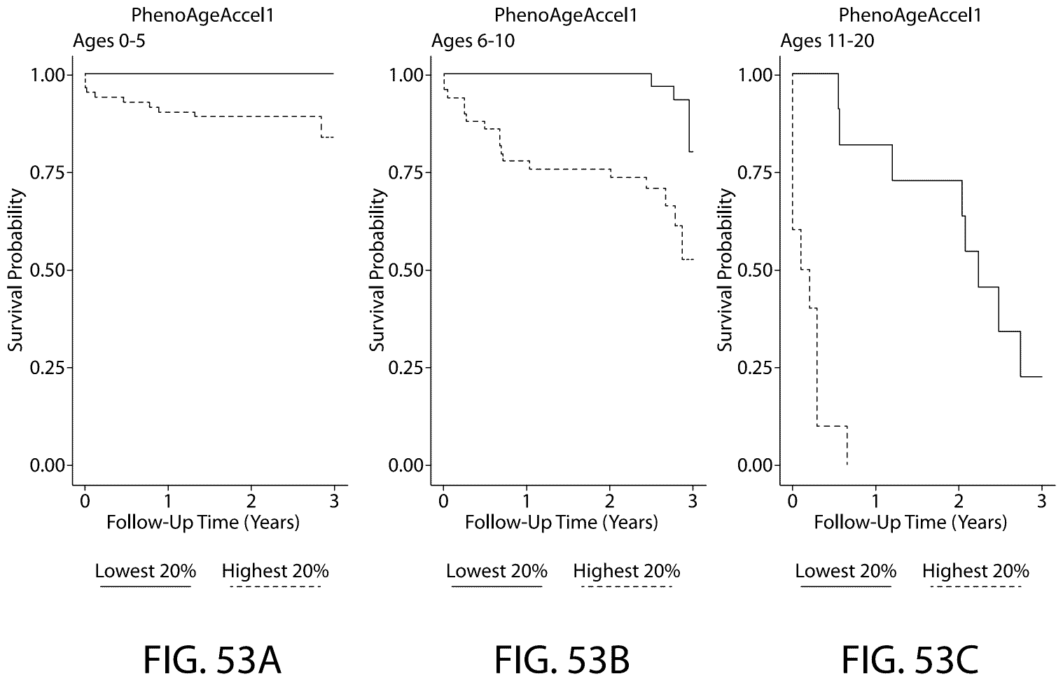

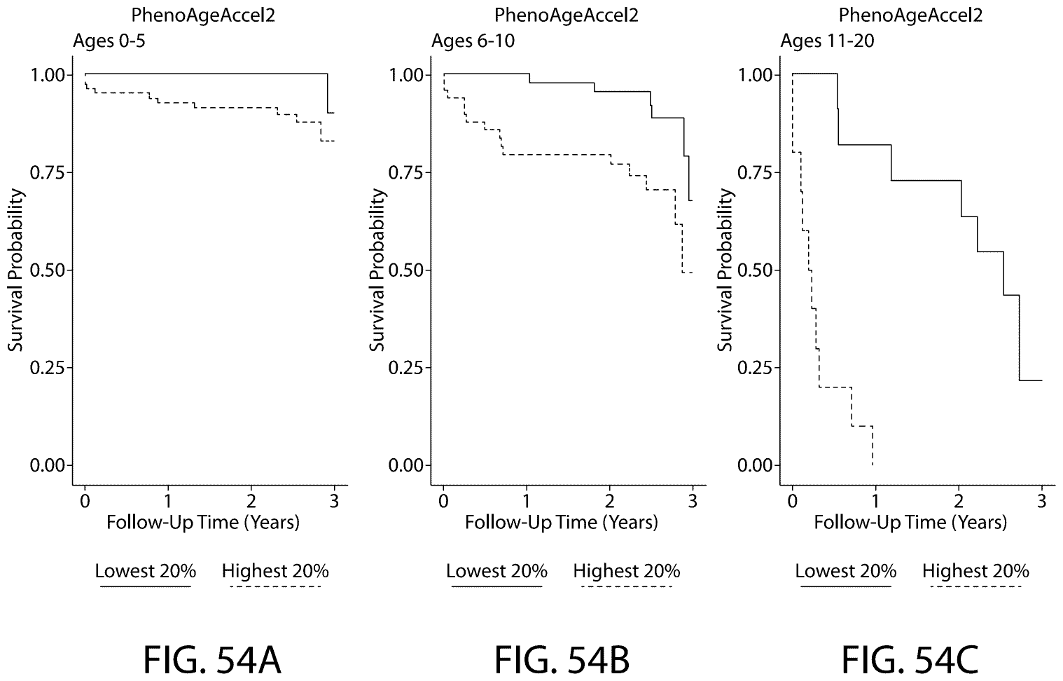

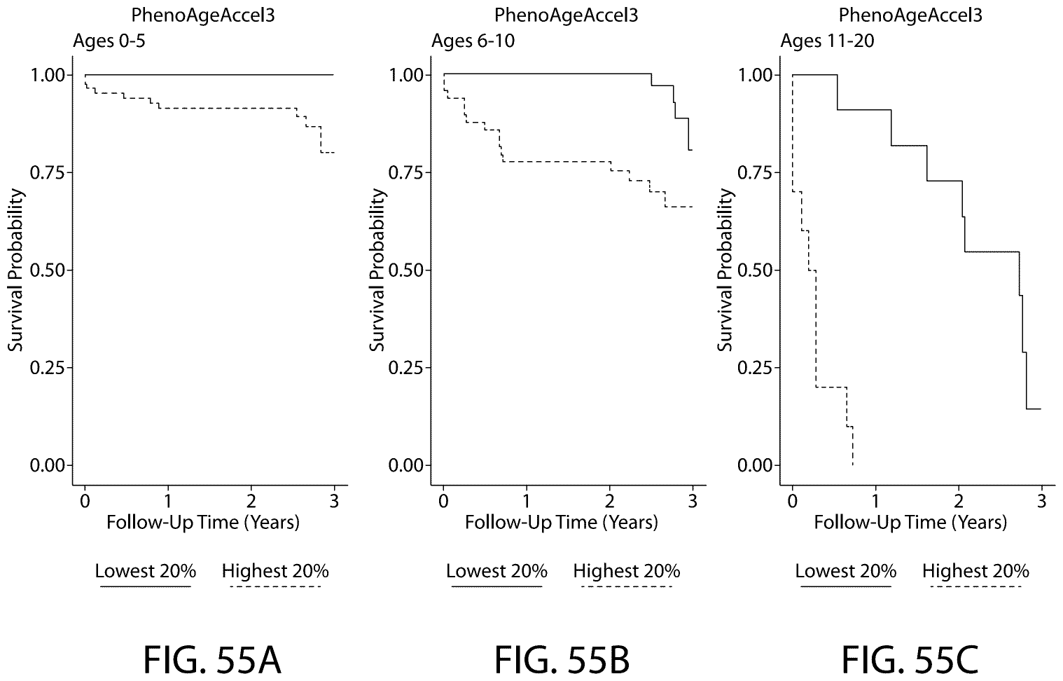

As shown in FIGS. 53A-53C, 54A-54C, and 55A-55C, canines with the highest Phenotypic Age relative to their chronological ages had steeper declines in survival over 3 years of follow-up, and differences grew across age intervals from canines ages 0-5 to canines ages 11-20 having the steepest decline in survival in the high risk group (highest 20% of PhenoAgeAccel). The difference in mortality between high and low risk PhenoAgeAccel is most predictive of mortality in ages 11-20, where high risk canines died within 1 year across models. The trends observed by age were observed for all models. In each of FIGS. 53A-55C, The y-axis indicates the survival rate, and the x-axis indicates follow-up time (in years).

### Phenotypic Age Acceleration

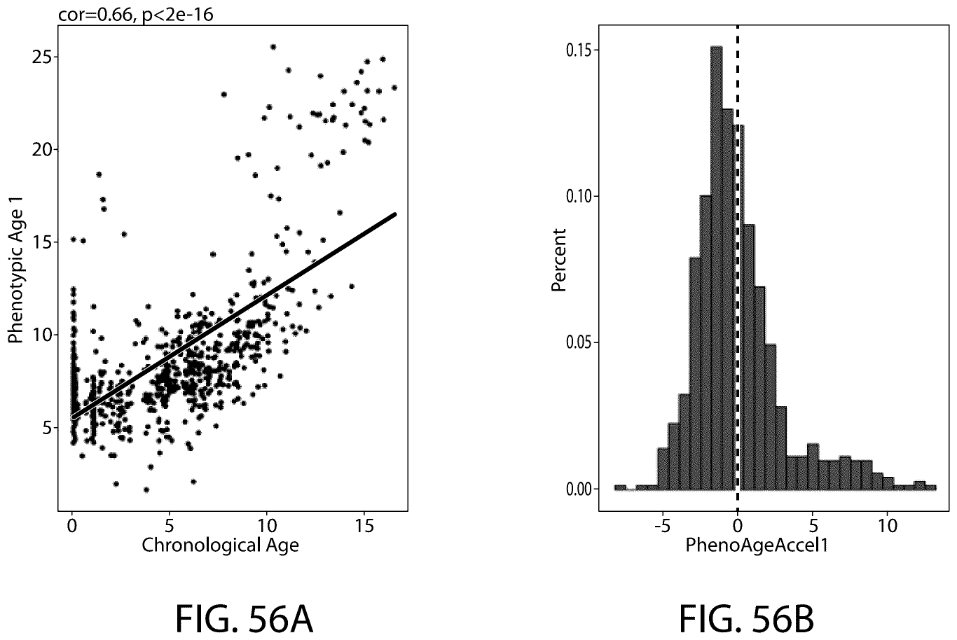

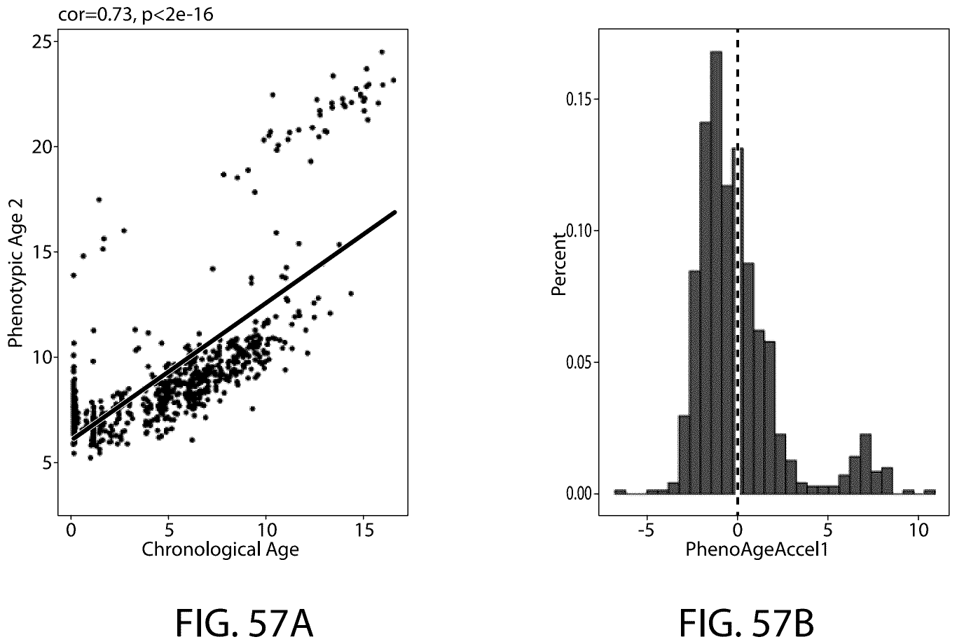

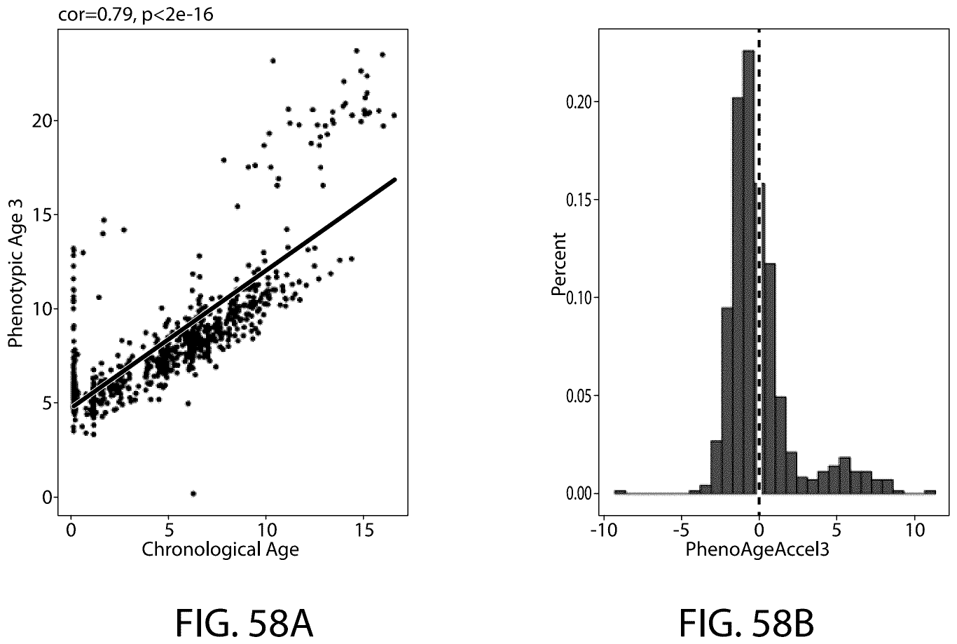

FIGS. 56A, 56B, 57A, 57B, 58A, and 58B illustrate the correlation between Phenotypic Age and chronological age, as well as the distribution of PhenoAgeAccel—the residual of Phenotypic Age regressed on chronological age. Phenotypic Age and chronological age are moderately correlated; part of this is due to the fact that age is in the Phenotypic Age measure. Phenotypic Age was moderately correlated with chronological age in Models 1 (r=0.66), 2 (r=0.73), and 3 (0.79) which is partially due to the fact that the models include chronological age. The red line depicts the expected Phenotypic Age for each chronological age, with points above the line depicting people who were phenotypically older than expected, and points below the line depicting those who were phenotypically younger than expected. PhenoAgeAccel was fairly normally distributed but had a wider range than observed in humans in Liu et al. in all models. Phenotypic Age was more strongly (r=0.79) correlated with chronological age in Model 3 relative to Models 1 and 2, similar to the feline analysis.

### 2-Year Mortality

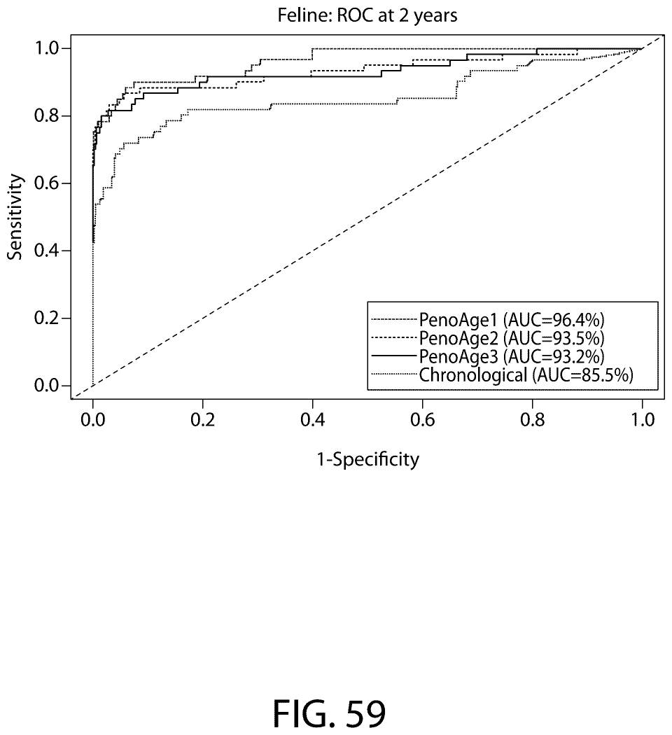

ROC curves in canines (FIG. 59) revealed that Phenotypic Age, with an area under the curve (AUC) of 0.964 in Model 1, 0.935 in Model 2, and 0.932 in Model 3 outperformed chronological age (0.855).

FIG. 59 illustrates receiver operating characteristic curves for 2-year mortality by model in canines.

### Discussion and Conclusions

Identifying reliable biomarkers of aging in canines and felines can promote healthy aging in these species. In this analysis, we leveraged methods from Levine et al. to estimate the PhenoAge of cohorts of felines and canines, evaluate the risk of mortality based on PhenoAge and the correlation between PhenoAge and chronological age. Our stepwise regression and Elastic Cox regression models more closely reflected predictors of mortality in the canine and feline populations than variables in the human population presented previously.

In line with previous studies in humans,[6-8] felines and canines with the highest Phenotypic Age relative to their chronological ages had steeper declines in survival over 3 years of follow-up, and differences grew across age intervals, with the steepest increases in mortality observed in the oldest age group (11-20 years) for both species.

Of note, Liu et al. reported a stronger correlation between PhenoAge and chronological age compared to other PhenoAge measures that were derived in our study. Yet, most of the variables from Liu's PhenoAge model were not associated with risk of death in our analysis. It seems that a variable selection method based on prior knowledge about aging-related variables may be more important than a data-driven variable selection method in animal populations.

Tables 12 and 13, below, show what was seen in animals if the models were built based on what the human based phenotypic age models (Liu, et al, performed in human populations). Phenotypic age models for cats and dogs are unique and cannot be easily transferred from models built for humans. According to the models developed here, many variables identified as predictive of death in humans do not predict death as strongly in cats and dogs.

TABLE 12
Association between Model 3 (using variables for Liu et al. model) and risk of mortality in felines
Analysis of Maximum Likelihood Estimates

| | | | | | | | 95% Hazard | |
|---|---|---|---|---|---|---|---|---|
| | | | | | | | Ratio | |
| | | Parameter | Standard | | | Hazard | Confidence | |
| Parameter | DF | Estimate | Error | Chi-Square | Pr > ChiSq | Ratio | Limits | |
| Creatinine | 1 | −0.08782 | 0.18827 | 0.2176 | 0.6409 | 0.916 | 0.633 | 1.325 |
| Glucose | 1 | 0.00313 | 0.00188 | 2.7807 | 0.0954 | 1.003 | 0.999 | 1.007 |
| MCV | 1 | −0.15041 | 0.02519 | 35.6382 | <.0001 | 0.860 | 0.819 | 0.904 |
| Albumin | 1 | 0.32704 | 0.29251 | 1.2500 | 0.2635 | 1.387 | 0.782 | 2.460 |
| RDW | 1 | 0.03111 | 0.02686 | 1.3410 | 0.2469 | 1.032 | 0.979 | 1.087 |
| Lymphocytes | 1 | 0.00618 | 0.00968 | 0.4073 | 0.5234 | 1.006 | 0.987 | 1.025 |
| WBC | 1 | 0.03175 | 0.01936 | 2.6894 | 0.1010 | 1.032 | 0.994 | 1.072 |
| ALP | 1 | 0.00263 | 0.00543 | 0.2343 | 0.6284 | 1.003 | 0.992 | 1.013 |
| animal_age_yrs | 1 | 0.28293 | 0.02574 | 120.7987 | <.0001 | 1.327 | 1.262 | 1.396 |

TABLE 13
Association between Model 3 (using variables for Liu et al. model) and risk of mortality in canines
Analysis of Maximum Likelihood Estimates

| | | | | | | | 95% Hazard | |
|---|---|---|---|---|---|---|---|---|
| | | | | | | | Ratio | |
| | | Parameter | Standard | | | Hazard | Confidence | |
| Parameter | DF | Estimate | Error | Chi-Square | Pr > ChiSq | Ratio | Limits | |
| Creatinine | 1 | −0.71684 | 0.53906 | 1.7683 | 0.1836 | 0.488 | 0.170 | 1.405 |
| Glucose | 1 | −0.00992 | 0.00922 | 1.1572 | 0.2820 | 0.990 | 0.972 | 1.008 |
| MCV | 1 | 0.01629 | 0.03978 | 0.1677 | 0.6822 | 1.016 | 0.940 | 1.099 |
| Albumin | 1 | −0.51971 | 0.26907 | 3.7307 | 0.0534 | 0.595 | 0.351 | 1.008 |
| RDW | 1 | 0.21008 | 0.02326 | 81.5376 | <.0001 | 1.234 | 1.179 | 1.291 |
| Lymphocytes | 1 | −0.02129 | 0.01704 | 1.5604 | 0.2116 | 0.979 | 0.947 | 1.012 |
| WBC | 1 | −0.01058 | 0.04110 | 0.0663 | 0.7968 | 0.989 | 0.913 | 1.072 |
| ALP | 1 | 0.0001433 | 0.0004145 | 0.1196 | 0.7295 | 1.000 | 0.999 | 1.001 |
| animal_age_yrs | 1 | 0.25118 | 0.03659 | 47.1117 | <.0001 | 1.286 | 1.197 | 1.381 |

Every reference cited in the above disclosure or in the below list is incorporated herein by reference as if fully set forth.

### REFERENCES

ADDIN EN.REFLIST 1. Poganik, J. R., et al., Biological age is increased by stress and restored upon recovery. Cell Metab, 2023. 35(5): p. 807-820.e5.
2. Lohman, T., et al., Predictors of Biological Age: The Implications for Wellness and Aging Research. Gerontol Geriatr Med, 2021. 7: p. 23337214211046419.
3. Husted, K. L. S., et al., A Model for Estimating Biological Age From Physiological Biomarkers of Healthy Aging: Cross-sectional Study. JMIR Aging, 2022. 5(2): p. e35696.
4. Raj, K., et al., Epigenetic clock and methylation studies in cats. Geroscience, 2021. 43(5): p. 2363-2378.
5. Jiménez, A. G., A revisiting of “the hallmarks of aging” in domestic dogs: current status of the literature. Geroscience, 2024. 46(1): p. 241-255.
6. Levine, M. E., et al., An epigenetic biomarker of aging for lifespan and healthspan. Aging (Albany NY), 2018. 10(4): p. 573-591.
7. Liu, Z., et al., A new aging measure captures morbidity and mortality risk across diverse subpopulations from NHANES IV: A cohort study. PLoS Med, 2018. 15(12): p. e1002718.
8. Liu, Z., et al., Correction: A new aging measure captures morbidity and mortality risk across diverse subpopulations from NHANES IV: A cohort study. PLoS Med, 2019. 16(2): p. e1002760.

What is claimed is:

1. A system for generating a multi-component aging index for an individual companion animal based on measuring at least one of digital biomarkers, traditional biomarkers and subjective assessment methods to predict phenotypic age and phenotypic age acceleration/deceleration in dogs and cats.

2. The system according to claim 1, the system further comprising at least one of
wearable devices to measure physical activity comprising walking, running, resting, jumping, sleep time, sleep quality and sleep regularity,
subjective assessment via at least one of pet parent questionnaires and veterinary questionnaires,
clinical characteristics comprising chronological age, weight, BCS, BFI, temperature, respiration rate, and heart rate,
environmental sensors comprising sensors detecting at least one of location and location-based behaviors and activities comprising proximity to pet parent, play, timing and frequency of feeding, location of eating, drinking, urination and defecation, body posture, pose estimation, tail position, body position, movement tracking over time,
repeated measures of clinical, digital and biological data,
eating, drinking, urinating, defecating patterns,
signs of emotional health, cognitive health, fear, anxiety, stress, dementia and social interaction with humans and other animals, and
veterinary assessment of at least one of gastrointestinal disease, genitourinary disease, kidney disease, dermatological disease, respiratory disease, neurological disease, muscular disease, ophthalmological disease, auditory disease, cardiovascular disease, cancer, oral health, endocrine disease, infectious disease, immune function, inflammation, orthopedic disease, mobility, and pain.

3. The system according to claim 2, wherein the wearable device is a Collar Mounted Activity Sensor (CMAS).

4. The system according to claim 1, wherein the biomarker panels comprise two or more traditional biomarkers selected from CBC/chemistry parameters, fecal microbiome, fecal metabolites, urinary microbiome, urinary metabolites, blood metabolites, and blood biomarkers comprising albumin, creatinine, glucose, lymphocyte percent, mean cell volume, red cell distribution width, alkaline phosphatase, white blood cell count, SDMA, circulating peptides comprising Aβ42, circulating postbiotics, immunoglobulins, immunoglobulin M, growth hormone (GH)/insulin growth factor-1 (IGF-1), and DNA biomarkers, SNPs, and genetic variants.

5. The system according to claim 4, wherein the DNA biomarkers comprise one or a plurality of single nucleotide polymorphism (SNPs).

6. The system according to claim 1, wherein the multi-component aging index for an individual companion animal is further based on measuring epigenetic modifications including the DNA methylome.

7. A method for decelerating phenotypic aging of a companion animal in need thereof comprising determining an index using the system of claim 1 and based thereon providing customized health, dietary and/or nutrition measures to the companion animal.

8. The method according to claim 7, wherein the companion animal is an overweight animal.

9. The method according to claim 7, wherein the companion animal is an undernourished animal.

10. The method according to claim 7, wherein the companion animal is an animal that has difficulty maintaining a healthy weight.

11. The method according to claim 7, wherein the personalized health measure comprises a diet that ameliorates accelerating phenotypic aging.

12. A system comprising:
(a) a biosensor comprising:
(i) a solid support comprising an internal cavity and external surface;
(i) a band operably linked to an external surface of the solid support;
(ii) an electrical circuit positioned within the internal cavity comprising at least a first position sensor and at least a first motion sensor;
(b) at least one computer storage memory; and
(c) a controller;
wherein each of the sensors is in electrical communication with the controller.

13. A biosensor comprising:
a top and a bottom exterior surface separated by a height, the exterior surface and the height defining an internal cavity comprising at least one of:
(i) a gyroscope;
(ii) at least a first pressure sensor;
(iii) at least a first temperature sensor;
(iv) at least a first accelerometer; and
(v) a controller,
wherein each of (i), (ii), (iii), and (iv) are in electrical communication with a controller by way of an electrical circuit.

14. The biosensor of claim 13, wherein the height is no more than about 3 inches.

15. The biosensor of claim 13, wherein the top and bottom exterior surfaces comprise a pliable materials chosen from: rubber, latex, vinyl or polyurethane, or a combination thereof.

16. The biosensor of claim 13, wherein the pressure sensor comprises at least one compression spring operably connected to the electrical circuit.

17. The biosensor of claim 13 further comprising a UV light spectrophotometer.

18. The biosensor of claim 13 further comprising a pH meter.

19. The biosensor of claim 13 further comprising a WiFi and Bluetooth communication antenna with a charging port.

20. The biosensor of claim 13 further comprising an amperometric hydrogen sulfide sensor.
